SEQUENCE LISTING (149 sequences)

SEQ ID NO: 1 - protein, 224 aa, Homo sapiens
IVGGYNCEENSVPYQVSLNSGYHFCGGSLINEQWVVSAGHCYKSRIQVRLGEHNIEVLEGNEQFINAAKIIRHPQYDRKTLNNDIMLIKLSSRAVINARVSTISLPTAPPATGTKCLISGWGNTASSGADYPDELQCLDAPVLSQAKCEASYPGKITSNMFCVGFLEGGKDSCQGDSGGPVVCNGQLQGVVSWGDGCAQKNKPGVYTKVYNYVKWIKNTIAANS

SEQ ID NO: 2 - protein, 235 aa, artificial sequence
  trypsin variant 1
IVGGYNCEENSVPYQVSLNSGYHFCGGSLINEQWVVSAGHCYDAVGRDKSRIQVRLGEHNIEVLEGNEQFINAAKIIRHPQYDRKTLNNDIMLIKLSSRAVINARVSTISLPTAPPATGTKCLISGWGNTITNSTASSGADYPDELQCLDAPVLSQAKCEASYPGKITSNMFCVGFLEGGKDSCQGDSGGPVVCNGQLQGVVSWGDGCAQKNKPGVYTKVYNYVKWIKNTIAANS

SEQ ID NO: 3 - protein, 235 aa, artificial sequence
  trypsin variant 2
IVGGYNCEENSVPYQVSLNSGYHFCGGSLINEQWVVSAGHCYNGRDLEKSRIQVRLGEHNIEVLEGNEQFINAAKIIRHPQYDRKTLNNDIMLIKLSSRAVINARVSTISLPTAPPATGTKCLISGWGNVRGTWTASSGADYPDELQCLDAPVLSQAKCEASYPGKITSNMFCVGFLEGGKDSCQGDSGGPVVCNGQLQGVVSWGDGCAQKNKPGVYTKVYNYVKWIKNTIAANS

SEQ ID NO: 4 - protein, 235 aa, artificial sequence
  trypsin variant 3
IVGGYNCEENSVPYQVSLNSGYHFCGGSLINEQWVVSAGHCYAATNGDKSRIQVRLGEHNIEVLEGNEQFINAAKIIRHPQYDRKTLNNDIMLIKLSSRAVINARVSTISLPTAPPATGTKCLISGWGNRKDFWTASSGADYPDELQCLDAPVLSQAKCEASYPGKITSNMFCVGFLEGGKDSCQGDSGGPVVCNGQLQGVVSWGDGCAQKNKPGVYTKVYNYVKWIKNTIAANS

SEQ ID NO: 5 - protein, 259 aa, Homo sapiens
IVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 6 - protein, 235 aa, Homo sapiens
IVGGSNAKEGAWPWVVGLYYGGRLLCGASLVSSDWLVSAAHCVYGRNLEPSKWTAILGLHMKSNLTSPQTVPRLIDEIVINPHYNRRRKDNDIAMMHLEFKVNYTDYIQPICLPEENQVFPPGRNCSIAGWGTVVYQGTTANILQEADVPLLSNERCQQQMPEYNITENMICAGYEEGGIDSCQGDSGGPLMCQENNRWFLAGVTSFGYKCALPNRPGVYARVSRFTEWIQSFLH

SEQ ID NO: 7 - protein, 275 aa, Bacillus subtilis
IAHEYAQSVPYGISQIKAPALHSQGYTGSNVKVAVIDSGIDSSHPDLNVRGGASFVPSETNPYQDGSSHGTHVAGTIAALNNSIGVLGVSPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINV

SEQ ID NO: 8 - protein, 320 aa, Murinae gen. sp.
VAKRRAKRDVYQEPTDPKFPQQWYLSGVTQRDLNVKEAWAQGFTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNADDWATNGVGRK

SEQ ID NO: 9 - protein, 330 aa, Homo sapiens
EKERSKRSALRDSALNLFNDPMWNQQWYLQDTRMTAALPKLDLHVIPVWQKGITGKGVVITVLDDGLEWNHTDIYANYDPEASYDFNDNDHDPFPRYDPTNENKHGTRCAGEIAMQANNHKCGVGVAYNSKVGGIRMLDGIVTDAIEASSIGFNPGHVDIYSASWGPNDDGKTVEGPGRLAQKAFEYGVKQGRQGKGSIFVWASGNGGRQGDNCDCDGYTDSIYTISISSASQQGLSPWYAEKCSSTLATSYSSGDYTDQRITSADLHNDCTETHTGTSASAPLAAGIFALALEANPNLTWRDMQHLVVWTSEYDPLANNPGWKKNGAGL

SEQ ID NO: 10 - protein, 297 aa, Homo sapiens
NTHPCQSDMNIEGAWKRGYTGKNIVVTILDDGIERTHPDLMQNYDALASCDVNGNDLDPMPRYDASNENKHGTRCAGEVAAAANNSHCTVGIAFNAKIGGVRMLDGDVTDMVEAKSVSFNPQHVHIYSASWGPDDDGKTVDGPAPLTRQAFENGVRMGRRGLGSVFVWASGNGGRSKDHCSCDGYTNSIYTISISSTAESGKKPWYLEECSSTLATTYSSGESYDKKIITTDLRQRCTDNHTGTSASAPMAAGIIALALEANPFLTWRDVQHVIVRTSRAGHLNANDWKTNAAGFKV

SEQ ID NO: 11 - protein, 328 aa, Homo sapiens
TLVDEQPLENYLDMEYFGTIGIGTPAQDFTVVFDTGSSNLWVPSVYCSSLACTNHNRFNPEDSSTYQSTSETVSITYGTGSMTGILGYDTVQVGGISDTNQIFGLSETEPGSFLYYAPFDGILGLAYPSISSSGATPVFDNIWNQGLVSQDLFSVYLSADDKSGSVVIFGGIDSSYYTGSLNWVPVTVEGYWQITVDSITMNGETIACAEGCQAIVDTGTSLLTGPTSPIANIQSDIGASENSDGDMVVSCSAISSLPDIVFTINGVQYPVPPSAYILQSEGSCISGFQGMNVPTESGELWILGDVFIRQYFTVFDRANNQVGLAPVA

SEQ ID NO: 12 - protein, 358 aa, Homo sapiens
EMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSA

SEQ ID NO: 13 - protein, 351 aa, Homo sapiens
PAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAA

SEQ ID NO: 14 - protein, 305 aa, Homo sapiens
MLEADDQGCIEEQGVEDSANEDSVDAKPDRSSFVPSLFSKKKKNVTMRSIKTTRDRVPTYQYNMNFEKLGKCIIINNKNFDKVTGMGVRNGTDKDAEALFKCFRSLGFDVIVYNDCSCAKMQDLLKKASEEDHTNAACFACILLSHGEENVIYGKDGVTPIKDLTAHFRGDRSKTLLEKPKLFFIQACRGTELDDGIQADSGPINDTDANPRYKIPVEADFLFAYSTVPGYYSWRSPGRGSWFVQALCSILEEHGKDLEIMQILTRVNDRVARHFESQSDDPHFHEKKQIPCVVSMLTKELYFSQ

SEQ ID NO: 15 - protein, 262 aa, Streptomyces sp. K15
VTKPTIAAVGGYAMNNGTGTTLYTKAADTRRSTGSTTKIMTAKVVLAQSNLNLDAKVTIQKAYSDYVVANNASQAHLIVGDKVTVRQLLYGLMLPSGCDAAYALADKYGSGSTRAARVKSFIGKMNTAATNLGLHNTHFDSFDGIGNGANYSTPRDLTKIASSAMKNSTFRTVVKTKAYTAKTVTKTGSIRTMDTWKNTNGLLSSYSGAIGVKTGSGPEAKYCLVFAATRGGKTVIGTVLASTSIPARESDATKIMNYGFAL

SEQ ID NO: 16 - protein, 256 aa, Human cytomegalovirus
MTMDEQQSQAVAPVYVGGFLARYDQSPDEAELLLPRDVVEHWLHAQGQGQPSLSVALPLNINHDDTAVVGHVAAMQSVRDGLFCLGCVTSPRFLEIVRRASEKSELVSRGPVSPLQPDKVVEFLSGSYAGLSLSSRRCDDVEQATSLSGSETTPFKHVALCSVGRRRGTLAVYGRDPEWVTQRFPDLTAADRDGLRAQWQRCGSTAVDASGDPFRSDSYGLLGNSVDALYIRERLPKLRYDKQLVGVTERESYVKA

SEQ ID NO: 17 - protein, 248 aa, Escherichia coli
VRSFIYEPFQIPSGSMMPTLLIGDFILVEKFAYGIKDPIYQKTLIETGHPKRGDIVVFKYPEDPKLDYIKRAVGLPGDKVTYDPVSKELTIQPGCSSGQACENALPVTYSNVEPSDFVQTFSRRNGGEATSGFFEVPKNETKENGIRLSERKETLGDVTHRILTVPIAQDQVGMYYQQPGQQLATWIVPPGQYFMMGDNRDNSADSRYWGFVPEANLVGRATAIWMSFDKQEGEWPTGLRLSRIGGIH

SEQ ID NO: 18 - protein, 317 aa, Serratia marcescens
MEQLRGLYPPLAAYDSGWLDTGDGHRIYWELSGNPNGKPAVFIHGGPGGGISPHHRQLFDPERYKVLLFDQRGCGRSRPHASLDNNTTWHLVADIERLREMAGVEQWLVFGGSWGSTLALAYAQTHPERVSEMVLRGIFTLRKQRLHWYYQDGASRFFPEKWERVLSILSDDERKDVIAAYRQRLTSADPQVQLEAAKLWSVWEGETVTLLPSRESASFGEDDFALAFARIENHYFTHLGFLESDDQLLRNVPLIRHIPAVIVHGRYDMACQVQNAWDLAKAWPEAELHIVEGAGHSYDEPGILHQLMIATDRFAGK

SEQ ID NO: 19 - protein, 229 aa, Escherichia coli
MELLLLSNSTLPGKAWLEHALPLIANQLNGRRSAVFIPFAGVTQTWDEYTDKTAEVLAPLGVNVTGIHRVADPLAAIEKAEIIIVGGGNTFQLLKESRERGLLAPMADRVKRGALYIGWSAGANLACPTIRTTNDMPIVDPNGFDALDLFPLQINPHFTNALPEGHKGETREQRIRELLVVAPELTVIGLPEGNWIQVSNGQAVLGGPNTTWVFKAGEEAVALEAGHRF

SEQ ID NO: 20 - protein, 99 aa, Human immunodeficiency virus
PQITLWQRPLVTVKIGGQLREALLDTGADDTVLEDINLPGKWKPKMIGGIGGFIKVRQYDQILIEICGKKAIGTVLVGPTPVNIIGRNMLTQIGCTLNF

SEQ ID NO: 21 - protein, 297 aa, Escherichia coli
STETLSFTPDNINADISLGTLSGKTKERVYLAEEGGRKVSQLDWKFNNAAIIKGAINWDLMPQISIGAAGWTTLGSRGGNMVDQDWMDSSNPGTWTDEARHPDTQLNYANEFDLNIKGWLLNEPNYRLGLMAGYQESRYSFTARGGSYIYSSEEGFRDDIGSFPNGERAIGYKQRFKMPYIGLTGSYRYEDFELGGTFKYSGWVESSDNDEHYDPKGRITYRSKVKDQNYYSVAVNAGYYVTPNAKVYVEGAWNRVTNKKGNTSLYDHNNNTSDYSKNGAGIENYNFITTAGLKYTF

SEQ ID NO: 22 - protein, 212 aa, Carica papaya
IPEYVDWRQKGAVTPVKNQGSCGSCWAFSAVVTIEGIIKIRTGNLNQYSEQELLDCDRRSYGCNGGYPWSALQLVAQYGIHYRNTYPYEGVQRYCRSREKGPYAAKTDGVRQVQPYNQGALLYSIANQPVSVVLQAAGKDFQLYRGGIFVGPCGNKVDHAVAAVGYGPNYILIKNSWGTGWGENGYIRIKRGTGNSYGVCGLYTSSFYPVKN

SEQ ID NO: 23 - protein, 699 aa, Homo sapiens
AGIAAKLAKDREAAEGLGSHERAIKYLNQDYEALRNECLEAGTLFQDPSFPAIPSALGFKELGPYSSKTRGMRWKRPTEICADPQFIIGGATRTDICQGALGDCWLLAAIASLTLNEEILARVVPLNQSFQENYAGIFHFQFWQYGEWVEVVVDDRLPTKDGELLFVHSAEGSEFWSALLEKAYAKINGCYEALSGGATTEGFEDFTGGIAEWYELKKPPPNLFKIIQKALQKGSLLGCSIDITSAADSEAITFQKLVKGHAYSVTGAEEVESNGSLQKLIRIRNPWGEVEWTGRWNDNCPSWNTIDPEERERLTRRHEDGEFWMSFSDFLRHYSRLEICNLTPDTLTSDTYKKWKLTKMDGNWRRGSTAGGCRNYPNTFWMNPQYLIKLEEEDEDEEDGESGCTFLVGLIQKHRRRQRKMGEDMHTIGFGIYEVPEELSGQTNIHLSKNFFLTNRARERSDTFINLREVLNRFKLPPGEYILVPSTFEPNKDGDFCIRVFSEKKADYQAVDDEIEANLEEFDISEDDIDDGVRRLFAQLAGEDAEISAFELQTILRRVLAKRQDIKSDGFSIETCKIMVDMLDSDGSGKLGLKEFYILWTKIQKYQKIYREIDVDRSGTMNSYEMRKALEEAGFKMPCQLHQVIVARFADDQLIIDFDNFVRCLVRLETLFKIFKQLDPENTGTIELDLISWLCFSVL

SEQ ID NO: 24 - protein, 221 aa, Tobacco etch virus
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMDKP

SEQ ID NO: 25 - protein, 371 aa, Streptococcus pyogenes
DQNFARNEKEAKDSAITFIQKSAAIKAGARSAEDIKLDKVNLGGELSGSNMYVYNISTGGFVIVSGDKRSPEILGYSTSGSFDVNGKENIASFMESYVEQIKENKKLDSTYAGTAEIKQPVVKSLLDSKGIHYNQGNPYNLLTPVIEKVKPGEQSFVGQHAATGSVATATAQIMKYHNYPNKGLKDYTYTLSSNNPYFNHPKNLFAAISTRQYNWNNILPTYSGRESNVQKMAISELMADVGISVDMDYGPSSGSAGSSRVQRALKENFGYNQSVHQINRGDFSKQDWEAQIDKELSQNQPVYYQGVGKVGGHAFVIDGADGRNFYHVNWGWGGVSDGFFRLDALNPSALGTGGGAGGFNGYQSAVVGIKP

SEQ ID NO: 26 - protein, 353 aa, Homo sapiens
KKHTGYVGLKNQGATCYMNSLLQTLFFTNQLRKAVYMMPTEGDDSSKSVPLALQRVFYELQHSDKPVGTKKLTKSFGWETLDSFMQHDVQELCRVLLDNVENKMKGTCVEGTIPKLFRGKMVSYIQCKEVDYRSDRREDYYDIQLSIKGKKNIFESFVDYVAVEQLDGDNKYDAGEHGLQEAEKGVKFLTLPPVLHLQLMRFMYDPQTDQNIKINDRFEFPEQLPLDEFLQKTDPKDPANYILHAVLVHSGDNHGGHYVVYLNPKGDGKWCKFDDDVVSRCTKEEAIEHNYGGHDDDLSVRHCTNAYMLVYIRESKLSEVLQAVTDHDIPQQLVERLQEEKRIEAQKRKERQE

SEQ ID NO: 27 - protein, 174 aa, Staphylococcus aureus
YNEQYVNKLENFKIRETQGNNGWCAGYTMSALLNATYNTNKYHAEAVMRFLHPNLQGQQFQFTGLTPREMIYFGQTQGRSPQLLNRMTTYNEVDNLTKNNKGIAILGSRVESRNGMHAGHAMAVVGNAKLNNGQEVIIIWNPWDNGFMTQDAKNNVIPVSNGDHYQWYSSIYGY

SEQ ID NO: 28 - protein, 221 aa, Saccharomyces cerevisiae
GSLVPELNEKDDDQVQKALASRENTQLMNRDNIEITVRDFKTLAPRRWLNDTIIEFFMKYIEKSTPNTVAFNSFFYTNLSERGYQGVRRWMKRKKTQIDKLDKIFTPINLNQSHWALGIIDLKKKTIGYVDSLSNGPNAMSFAILTDLQKYVMEESKHTIGEDFDLIHLDCPQQPNGYDCGIYVCMNTLYGSADAPLDFDYKDAIRMRRFIAHLILTDALK

SEQ ID NO: 29 - protein, 166 aa, Pyrococcus horikoshii
MKVLFLTANEFEDVELIYPYHRLKEEGHEVYIASFERGTITGKHGYSVKVDLTFDKVNPEEFDALVLPGGRAPERVRLNEKAVSIARKMFSEGKPVASICHGPQILISAGVLRGRKGTSYPGIKDDMINAGVEWVDAEVVVDGNWVSSRVPADLYAWMREFVKLLK

SEQ ID NO: 30 - protein, 316 aa, Bacillus thermoproteolyticus [sequence omitted]

SEQ ID NO: 31 - protein, 169 aa, Homo sapiens
VLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQP

SEQ ID NO: 32 - protein, 496 aa, Homo sapiens
QYSPNTQQGRTSIVHLFEWRWVDIALECERYLAPKGFGGVQVSPPNENVAIYNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLTGLLDLALEKDYVRSKIAEYMNHLIDIGVAGFRLDASKHMWPGDIKAILDKLHNLNSNWFPAGSKPFIYQEVIDLGGEPIKSSDYFGNGRVTEFKYGAKLGTVIRKWNGEKMSYLKNWGEGWGFVPSDRALVFVDNHDNQRGHGAGGASILTFWDARLYKMAVGFMLAHPYGFTRVMSSYRWPRQFQNGNDVNDWVGPPNNNGVIKEVTINPDTTCGNDWVCEHRWRQIRNMVIFRNVVDGQPFTNWYDNGSNQVAFGRGNRGFIVFNNDDWSFSLTLQTGLPAGTYCDVISGDKINGNCTGIKIYVSDDGKAHFSISNSAEDPFIAIHAESKL

SEQ ID NO: 33 - protein, 370 aa, Trichoderma reesei
QPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTT

SEQ ID NO: 34 - protein, 223 aa, Aspergillus niger
QTMCSQYDSASSPPYSVNQNLWGEYQGTGSQCVYVDKLSSSGASWHTEWTWSGGEGTVKSYSNSGVTFNKKLVSDVSSIPTSVEWKQDNTNVNADVAYDLFTAANVDHATSSGDYELMIWLARYGNIQPIGKQIATATVGGKSWEVWYGSTTQAGAEQRTYSFVSESPINSYSGDINAFFSYLTQNQGFPASSQYLINLQFGTEAFTGGPATFTVDNWTASVN

SEQ ID NO: 35 - protein, 184 aa, Aspergillus niger
SAGINYVQNYNGNLGDFTYDESAGTFSMYWEDGVSSDFVVGLGWTTGSSNAITYSAEYSASGSASYLAVYGWVNYPQAEYYIVEDYGDYNPCSSATSLGTVYSDGSTYQVCTDTRTNEPSITGTSTFTQYFSVRESTRTSGTVTVANHFNFWAHHGFGNSDFNYQVVAVEAWSGAGSASVTISS

SEQ ID NO: 36 - protein, 313 aa, Streptomyces lividans
AESTLGAAAAQSGRYFGTAIASGRLSDSTYTSIAGREFNMVTAENEMKIDATEPQRGQFNFSSADRVYNWAVQNGKQVRGHTLAWHSQQPGWMQSLSGSALRQAMIDHINGVMAHYKGKIVQWDVVNEAFADGSSGARRDSNLQRSGNDWIEVAFRTARAADPSAKLCYNDYNVENWTWAKTQAMYNMVRDFKQRGVPIDCVGFQSHFNSGSPYNSNFRTTLQNFAALGVDVAITELDIQGAPASTYANVTNDCLAVSRCLGITVWGVRDSDSWRSEQTPLLFNNDGSKKAAYTAVLDALNGGASSEPPADGG

SEQ ID NO: 37 - protein, 362 aa, Aspergillus niger
MHSFASLLAYGLVAGATFASASPIEARDSCTFTTAAAAKAGKAKCSTITLNNIEVPAGTTLDLTGLTSGTKVIFEGTTTFQYEEWAGPLISMSGEHITVTGASGHLINCDGARWWDGKGTSGKKKPKFFYAHGLDSSSITGLNIKNTPLMAFSVQANDITFTDVTINNADGDTQGGHNTDAFDVGNSVGVNIIKPWVHNQDDCLAVNSGENIWFTGGTCIGGHGLSIGSVGDRSNNVVKNVTIEHSTVSNSENAVRIKTISGATGSVSEITYSNIVMSGISDYGVVIQQDYEDGKPTGKPTNGVTIQDVKLESVTGSVDSGATEIYLLCGSGSCSDWTWDDVKVTGGKKSTACKNFPSVASC

SEQ ID NO: 38 - protein, 383 aa, Pseudomonas cellulosa
RADVKPVTVKLVDSQATMETRSLFAFMQEQRRHSIMFGHQHETTQGLTITRTDGTQSDTFNAVGDFAAVYGWDTLSIVAPKAEGDIVAQVKKAYARGGIITVSSHFDNPKTDTQKGVWPVGTSWDQTPAVVDSLPGGAYNPVLNGYLDQVAEWANNLKDEQGRLIPVIFRLYHANTGSWFWWGDKQSTPEQYKQLFRYSVEYLRDVKGVRNFLYAYSPNNFWDVTEANYLERYPGDEWVDVLGFDTYGPVADNADWFRNVVANAALVARMAEARGKIPVISEIGIRAPDIEAGLYDNQWYRKLISGLKADPDAREIAFLLVWRNAPQGVPGPNGTQVPHYWVPANRPENINNGTLEDFQAFYADEFTAFNRDIEQVYQRPTLI

SEQ ID NO: 39 - protein, 419 aa, Bacillus circulans
LQPATAEAADSYKIVGYYPSWAAYGRNYNVADIDPTKVTHINYAFADICWNGIHGNPDPSGPNPVTWTCQNEKSQTINVPNGTIVLGDPWIDTGKTFAGDTWDQPIAGNINQLNKLKQTNPNLKTIISVGGWTWSNRFSDVAATAATREVFANSAVDFLRKYNFDGVDLDWEYPVSGGLDGNSKRPEDKQNYTLLLSKIREKLDAAGAVDGKKYLLTIASGASATYAANTELAKIAAIVDWINIMTYDFNGAWQKISAHNAPLNYDPAASAAGVPDANTFNVAAGAQGHLDAGVPAAKLVLGVPFYGRGWDGCAQAGNGQYQTCTGGSSVGTWEAGSFDFYDLEANYINKNGYTRYWNDTAKVPYLYNASNKRFISYDDAESVGYKTAYIKSKGLGGAMFWELSGDRNKTLQNKLKADL

SEQ ID NO: 40 - protein, 317 aa, Candida antarctica [sequence omitted]

SEQ ID NO: 41 - protein, 434 aa, artificial sequence
  chimera of guinea pig and homo sapiens (human= approx. last 30 amino acids)
AEVCYSHLGCFSDEKPWAGTSQRPIKSLPSDPKKINTRFLLYTNENQNSYQLITATDIATIKASNFNLNRKTRFIIHGFTDSGENSWLSDMCKNMFQVEKVNCICVDWKGGSKAQYSQASQNIRVVGAEVAYLVQVLSTSLNYAPENVHIIGHSLGAHTAGEAGKRLNGLVGRITGLDPAEPYFQDTPEEVRLDPSDAKFVDVIHTDISPILPSLGFGMSQKVGHMDFFPNGGKDMPGCKTGISCNHHRSIEYYHSSILNPEGFLGYPCASYDEFQESGCFPCPAKGCPKMGHFADQYPGKTNAVEQTFFLNTGASDNFTRWRYKVTVTLSGEKDPSGNINVALLGKNGNSAQYQVFKGTLKPDASYTNSIDVELNVGTIQKVTFLWKRSGISVSKPKMGASRITVQSGKDGTKYNFCSSDIVQENVEQTLSPC

SEQ ID NO: 42 - protein, 471 aa, Escherichia coli
MKQSTIALALLPLLFTPVTKARTPEMPVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 43 - protein, 260 aa, Bovine
LKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 44 - protein, 686 aa, Bacillus circulans
APDTSVSNKQNFSTDVIYQIFTDRFSDGNPANNPTGAAFDGTCTNLRLYCGGDWQGIINKINDGYLTGMGVTAIWISQPVENIYSIINYSGVNNTAYHGYWARDFKKTNPAYGTIADFQNLIAAAHAKNIKVIIDFAPNHTSPASSDQPSFAENGRLYDNGTLLGGYTNDTQNLFHHNGGTDFSTTENGIYKNLYDLADLNHNNSTVDVYLKDAIKMWLDLGIDGIRMDAVKHMPFGWQKSFMAAVNNYKPVFTFGEWFLGVNEVSPENHKFANESGMSLLDFRFAQKVRQVFRDNTDNMYGLKAMLEGSAADYAQVDDQVTFIDNHDMERFHASNANRRKLEQALAFTLTSRGVPAIYYGTEQYMSGGTDPDNRARIPSFSTSTTAYQVIQKLAPLRKCNPAIAYGSTQERWINNDVLIYERKFGSNVAVVAVNRNLNAPASISGLVTSLPQGSYNDVLGGLLNGNTLSVGSGGAASNFTLAAGGTAVWQYTAATATPTIGHVGPMMAKPGVTITIDGRGFGSSKGTVYFGTTAVSGADITSWEDTQIKVKIPAVAGGNYNIKVANAAGTASNVYDNFEVLSGDQVSVRFVVNNATTALGQNVYLTGSVSELGNWDPAKAIGPMYNQVVYQYPNWYYDVSVPAGKTIEFKFLKKQGSTVTWEGGSNHTFTAPSSGTATINVNWQP

SEQ ID NO: 45 - protein, 404 aa, Amycolatopsis orientalis
MRVLITGCGSRGDTEPLVALAARLRELGADARMCLPPDYVERCAEVGVPMVPVGRAVRAGAREPGELPPGAAEVVTEVVAEWFDKVPAAIEGCDAVVTTGLLPAAVAVRSMAEKLGIPYRYTVLSPDHLPSEQSQAERDMYNQGADRLFGDAVNSHRASIGLPPVEHLYDYGYTDQPWLAADPVLSPLRPTDLGTVQTGAWILPDERPLSAELEAFLAAGSTPVYVGFGSSSRPATADAAKMAIKAVRASGRRIVLSRGWADLVLPDDGADCFVVGEVNLQELFGRVAAAIHHDSAGTTLLAMRAGIPQIVVRRVVDNVVEQAYHADRVAELGVGVAVDGPVPTIDSLSAALDTALAPEIRARATTVADTIRADGTTVAAQLLFDAVSLEKPTVPALEHHHHHH

SEQ ID NO: 46 - protein, 292 aa, Pseudomonas sp.
SIERLGYLGFAVKDVPAWDHFLTKSVGLMAAGSAGDAALYRADQRAWRIAVQPGELDDLAYAGLEVDDAAALERMADKLRQAGVAFTRGDEALMQQRKVMGLLCLQDPFGLPLEIYYGPAEIFHEPFLPSAPVSGFVTGDQGIGHFVRCVPDTAKAMAFYTEVLGFVLSDIIDIQMGPETSVPAHFLHCNGRHHTIALAAFPIPKRIHHFMLQANTIDDVGYAFDRLDAAGRITSLLGRHTNDQTLSFYADTPSPMIEVEFGWGPRTVDSSWTVARHSRTAMWGHKSVRGQR

SEQ ID NO: 47 - protein, 311 aa, Acitenobacter sp.
MEVKIFNTQDVQDFLRVASGLEQEGGNPRVKQIIHRVLSDLYKAIEDLNITSDEYWAGVAYLNQLGANQEAGLLSPGLGFDHYLDMRMDAEDAALGIENATPRTIEGPLYVAGAPESVGYARMDDGSDPNGHTLILHGTIFDADGKPLPNAKVEIWHANTKGFYSHFDPTGEQQAFNMRRSIITDENGQYRVRTILPAGYGCPPEGPTQQLLNQLGRHGNRPAHIHYFVSADGHRKLTTQINVAGDPYTYDDFAYATREGLVVDAVEHTDPEAIKANDVEGPFAEMVFDLKLTRLVDGVDNQVVDRPRLAV

SEQ ID NO: 48 - protein, 414 aa, Pseudomonas putida
TTETIQSNANLAPLPPHVPEHLVFDFDMYNPSNLSAGVQEAWAVLQESNVPDLVWTRCNGGHWIATRGQLIREAYEDYRHFSSECPFIPREAGEAYDFIPTSMDPPEQRQFRALANQVVGMPVVDKLENRIQELACSLIESLRPQGQCNFTEDYAEPFPIRIFMLLAGLPEEDIPHLKYLTDQMTRPDGSMTFAEAKEALYDYLIPIIEQRRQKPGTDAISIVANGQVNGRPITSDEAKRMCGLLLVGGLDTVVNFLSFSMEFLAKSPEHRQELIQRPERIPAACEELLRRFSLVADGRILTSDYEFHGVQLKKGDQILLPQMLSGLDERENACPMHVDFSRQKVSHTTFGHGSHLCLGQHLARREIIVTLKEWLTRIPDFSIAPGAQIQHKSGIVSGVQALPLVWDPATTKAV

SEQ ID NO: 49 - protein, 374 aa, Equus caballus
STAGKVIKCKAAVLWEEKKPFSIEEVEVAPPKAHEVRIKMVATGICRSDDHVVSGTLVTPLPVIAGHEAAGIVESIGEGVTTVRPGDKVIPLFTPQCGKCRVCKHPEGNFCLKNDLSMPRGTMQDGTSRFTCRGKPIHHFLGTSTFSQYTVVDEISVAKIDAASPLEKVCLIGCGFSTGYGSAVKVAKVTQGSTCAVFGLGGVGLSVIMGCKAAGAARIIGVDINKDKFAKAKEVGATECVNPQDYKKPIQEVLTEMSNGGVDFSFEVIGRLDTMVTALSCCQEAYGVSVIVGVPPDSQNLSMNPMLLLSGRTWKGAIFGGFKSKDSVPKLVADFMAKKFALDPLITHVLPFEKINEGFDLLRSGESIRTILTF

SEQ ID NO: 50 - protein, 297 aa, Escherichia coli
MATNLRGVMAALLTPFDQQQALDKASLRRLVQFNIQQGIDGLYVGGSTGEAFVQSLSEREQVLEIVAEEGKGKIKLIAHVGCVTTAESQQLAASAKRYGFDAVSAVTPFYYPFSFEEHCDHYRAIIDSADGLPMVVYNIPALSGVKLTLDQINTLVTLPGVGALKQTSGDLYQMEQIRREHPDLVLYNGYDEIFASGLLAGADGGIGSTYNIMGWRYQGIVKALKEGDIQTAQKLQTECNKVIDLLIKTGVFRGLKTVLHYMDVVSVPLCRKPFGPVDEKYLPELKALAQQLMQERG

SEQ ID NO: 51 - protein, 268 aa, Salmonella typhimurium
MERYENLFAQLNDRREGAFVPFVTLGDPGIEQSLKIIDTLIDAGADALELGVPFSDPLADGPTIQNANLRAFAAGVTPAQCFEMLALIREKHPTIPIGLLMYANLVFNNGIDAFYARCEQVGVDSVLVADVPVEESAPFRQAALRHNIAPIFICPPNADDDLLRQVASYGRGYTYLLSRSGVTGAENRGALPLHHLIEKLKEYHAAPALQGFGISSPEQVSAAVRAGAAGAISGSAIVKIIEKNLASPKQMLAELRSFVSAMKAASRA

SEQ ID NO: 52 - protein, 393 aa, Actinoplanes missouriensis
SVQATREDKFSFGLWTVGWQARDAFGDATRTALDPVEAVHKLAEIGAYGITFHDDDLVPFGSDAQTRDGIIAGFKKALDETGLIVPMVTTNLFTHPVFKDGGFTSNDRSVRRYAIRKVLRQMDLGAELGAKTLVLWGGREGAEYDSAKDVSAALDRYREALNLLAQYSEDRGYGLRFAIEPKPNEPRGDILLPTAGHAIAFVQELERPELFGINPETGNEQMSNLNFTQGIAQALWHKKLFHIDLNGQHGPKFDQDLVFGHGDLLNAFSLVDLLENGPDGAPAYDGPRHFDYKPSRTEDYDGVWESAKANIRMYLLLKERAKAFRADPEVQEALAASKVAELKTPTLNPGEGYAELLADRSAFEDYDADAVGAKGFGFVKLNQLAIEHLLGAR

SEQ ID NO: 53 - protein, 348 aa, Bacteriophage T7
VNIKTNPFKAVSFVESAIKKALDNAGYLIAEIKYDGVRGNICVDNTANSYWLSRVSKTIPALEHLNGFDVRWKRLLNDDRCFYKDGFMLDGELMVKGVDFNTGSGLLRTKWTDTKNQEFHEELFVEPIRKKDKVPFKLHTGHLHIKLYAILPLHIVESGEDCDVMTLLMQEHVKNMLPLLQEYFPEIEWQAAESYEVYDMVELQQLYEQKRAEGHEGLIVKDPMCIYKRGKKSGWWKMKPENEADGIIQGLVWGTKGLANEGKVIGFEVLLESGRLVNATNISRALMDEFTETVKEATLSQWGFFSPYGIGDNDACTINPYDGWACQISYMEETPDGSLRHPSFVMFR

SEQ ID NO: 54 - DNA, 42 bp, artificial sequence
  binding site for restr1 and restr2
ggtggtatcagcaggccactgctacaagtcccgcatccaggt

SEQ ID NO: 55 - protein, 13 aa, artificial sequence
  binding site for restr1 and restr2
VVSAGHCYKSRIQ

SEQ ID NO: 56 - DNA, 42 bp, artificial sequence
  forward primer restr1
ggtggtatccgcgggccactgctacaagtcccggatccaggt

SEQ ID NO: 57 - DNA, 42 bp, artificial sequence
  reverse primer restr2
acctggatccgggacttgtagcagtggcccgcggataccacc

SEQ ID NO: 58 - DNA, 50 bp, artificial sequence
  binding site for restr3 and restr4
ccactggcacgaagtgcctcatctctggctggggcaacactgcgagctct

SEQ ID NO: 59 - protein, 16 aa, artificial sequence
  binding site for restr3 and restr4
TGTKCLISGWGNTASS

SEQ ID NO: 60 - DNA, 50 bp, artificial sequence; identical to SEQ ID NO: 58
  forward primer restr3

SEQ ID NO: 61 - DNA, 50 bp, artificial sequence
  reverse primer restr4
agagctagcagtgttgccccagccagagatgaggcacttggtaccagtgg

SEQ ID NO: 62 - DNA, 30 bp, artificial sequence
  primer puc-forward
ggggtaccccaccaccatgaatccactcct

SEQ ID NO: 63 - DNA, 30 bp, artificial sequence
  primer puc-reverse
cgggatccggtatagagactgaagagatac

SEQ ID NO: 64 - DNA, 39 bp, artificial sequence
  oligox-SDR1f
gggccactgctacnnnnnnnnnnnnnnnnnnaagtcccg

SEQ ID NO: 65 - protein, 12 aa, artificial sequence
  misc_feature (5)..(5): The ′Xaa′ at location 5 stands for Lys, Asn, Arg, Ser, Thr, Ile, Met, Glu, Asp, Gly, Ala, Val, Gln, His, Pro, Leu, a stop codon, Tyr, Trp, Cys, or Phe.
GHCYXXXXXXKS

SEQ ID NO: 66 - DNA, 45 bp, artificial sequence
  oligox-SDR1r
cgcccggtgacgatgnnnnnnnnnnnnnnnnnnttcagggcctag

SEQ ID NO: 67 - DNA, 47 bp, artificial sequence
  oligox-SDR2f
caagtgcctcatctctggctggggcaacnnnnnnnnnnnnnnnactg

SEQ ID NO: 68 - protein, 15 aa, artificial sequence
  misc_feature (10)..(10): The ′Xaa′ at location 10 stands for Lys, Asn, Arg, Ser, Thr, Ile, Met, Glu, Asp, Gly, Ala, Val, Gln, His, Pro, Leu, a stop codon, Tyr, Trp, Cys, or Phe.
KCLISGWGNXXXXXT

SEQ ID NO: 69 - DNA, 55 bp, artificial sequence
  oligox-SDR2r
catggttcacggagtagagaccgaccccgttgnnnnnnnnnnnnnnntgacgatc

SEQ ID NO: 70 - DNA, 59 bp, artificial sequence
  primer SDR1-mutnnb-forward
tggtatccgcgggccactgctacnnbnnbnnbnnbnnbnnbaagtcccggatccaggtg

SEQ ID NO: 71 - DNA, 52 bp, artificial sequence
  primer SDR2-mutnnb-reverse
ggcgccagagctagcagtvnnvnnvnnvnnvnngttgccccagccagagatg

SEQ ID NO: 72 - protein, 6 aa, artificial sequence
  variant g SDR1
AFFNGD

SEQ ID NO: 73 - protein, 5 aa, artificial sequence
  variant g SDR2
RKDPW

SEQ ID NO: 74 - protein, 234 aa, artificial sequence
  artificial sequence
IVGGYNCEENSVPYQVSLNSGYHFCGGSLINEQWVVSAGHCYAAFNGKSRIQVRLGEHNIEVLEGNEQFINAAKIIRHPQYDRKTLNNDIMLIKLSSRAVINARVSTISLPTAPPATGTKCLISGWGNRKDFWTASSGADYPDELQCLDAPVLSQAKCEASYPGKITSNMFCVGFLEGGKDSCQGDSGGPVVCNGQLQGVVSWGDGCAQKNKPGVYTKVYNYVKWIKNTIAANS

SEQ ID NO: 75 - protein, 234 aa, artificial sequence
  artificial sequence
IVGGYNCEENSVPYQVSLNSGYHFCGGSLINEQWVVSAGHCYAAFNGKSRIQVRLGEHNIGVLEGNEQFINAAKIIRHPQYDWKTLNNDIMLIKLSSRAVINARVSTISLPTAPPATGTKCLISGWGNRKDFWTASSGADFPDELQCLDAPVLSQTKCEASYPGKITSNMFCVGFLEGGKDSCQGDSGGPVVRNGQLQGVVSWGDGCAQKNKPGVYTKVYNYVKWIKNTIAANS

SEQ ID NO: 76 - protein, 12 aa, artificial sequence
  substrate A
LLWLGRVVGGPV

SEQ ID NO: 77 - protein, 12 aa, artificial sequence
  substrate B
KKWLGRVPGGPV

SEQ ID NO: 78 - protein, 6 aa, artificial sequence
  variant1 SDR1
DAVGRD

SEQ ID NO: 79 - protein, 6 aa, artificial sequence
  variant2 SDR1
NGRDLE

SEQ ID NO: 80 - protein, 6 aa, artificial sequence
  variant3 SDR1
GFVMFN

SEQ ID NO: 81 - protein, 5 aa, artificial sequence
  variant1 SDR2
RVHPS

SEQ ID NO: 82 - protein, 5 aa, artificial sequence
  variant2 SDR2
VRGTW

SEQ ID NO: 83 - protein, 5 aa, artificial sequence
  variant3 SDR2
RSPLT

SEQ ID NO: 84 - protein, 6 aa, artificial sequence
  variant a SDR1
RPWDPS

SEQ ID NO: 85 - protein, 6 aa, artificial sequence
  variant b SDR1
GFVMFN

SEQ ID NO: 86 - protein, 6 aa, artificial sequence
  variant c SDR1
EIANRE

SEQ ID NO: 87 - protein, 6 aa, artificial sequence
  variant d SDR1
KAVVGT

SEQ ID NO: 88 - protein, 6 aa, artificial sequence
  variant e SDR1
VNIMAA

SEQ ID NO: 89 - protein, 6 aa, artificial sequence
  variant f SDR1
AAFNGD

SEQ ID NO: 90 - protein, 5 aa, artificial sequence
  variant a SDR2
VHPTS

SEQ ID NO: 91 - protein, 5 aa, artificial sequence
  variant b SDR2
RSPLT

SEQ ID NO: 92 - protein, 5 aa, artificial sequence
  variant c SDR2
RGART

SEQ ID NO: 93 - protein, 5 aa, artificial sequence
  variant d SDR2
RTPIS

SEQ ID NO: 94 - protein, 5 aa, artificial sequence
  variant d SDR2
TTARK

SEQ ID NO: 95 - protein, 5 aa, artificial sequence
  variant f SDR2
RKDFW

SEQ ID NO: 96 - protein, 157 aa, Homo sapiens
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLLFAESGQVYFGIIAL

SEQ ID NO: 97 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 98 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 99 - protein, 133 aa, Homo sapiens [sequence omitted]

SEQ ID NO: 100 - protein, 72 aa, Homo sapiens
SAKELRCQCIKTYSKPFHPKFIKELRVIESGPHCANTEIIVKLSDGRELCLDPKENWVQRVVEKFLKRAENS

SEQ ID NO: 101 - protein, 74 aa, Homo sapiens
GPASVPTTCCFNLANRKIPLQRLESYRRITSGKCPQKAVIFKTKLAKDICADPKKKWVQDSMKYLDQKSPTPKP

SEQ ID NO: 102 - protein, 76 aa, Homo sapiens
QPDAINAPVTCCYNFTNRKISVQRLASYRRITSSKCPKEAVIFKTIVAKEICADPKQKWVQDSMDHLDKQTQTPKT

SEQ ID NO: 103 - protein, 206 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 104 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 105 - protein, 30 aa, Homo sapiens
FVNQHLCGSHLVEALYLVCGERGFFYTPKT

SEQ ID NO: 106 - protein, 21 aa, Homo sapiens
GIVEQCCTSICSLYQLENYCN

SEQ ID NO: 107 - protein, 28 aa, Homo sapiens
GSSFLSPEHQRVQQRKESKKPPAKLQPR

SEQ ID NO: 108 - protein, 9 aa, Homo sapiens
RVYIHPFHL

SEQ ID NO: 109 - protein, 114 aa, Homo sapiens
PMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA

SEQ ID NO: 110 - protein, 425 aa, Homo sapiens
MGPRRLLLVAACFSLCGPLLSARTRARRPESKATNATLDPRSFLLRNPNDKYEPFWEDEEKNESGLTEYRLVSINKSSPLQKQLPAFISEDASGYLTSSWLTLFVPSVYTGVFVVSLPLNIMAIVVFILKMKVKKPAVVYMLHLATADVLFVSVLPFKISYYFSGSDWQFGSELCRFVTAAFYCNMYASILLMTVISIDRFLAVVYPMQSLSWRTLGRASFTCLAIWALAIAGVVPLLLKEQTIQVPGLNITTCHDVLNETLLEGYYAYYFSAFSAVFFFVPLIISTVCYVSIIRCLSSSAVANRSKKSRALFLSAAVFCIFIICFGPTNVLLIAHYSFLSHTSTTEAAYFAYLLCVCVSSISCCIDPLIYYYASSECQRYVYSILCCKESSDPSSYNSSGQLMASKMDTCSSNLNNSIYKKLLT

SEQ ID NO: 111 - protein, 397 aa, Homo sapiens
MRSPSAAWLLGAAILLAASLSCSGTIQGTNRSSKGRSLIGKVDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 112 - protein, 153 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 113 - protein, 385 aa, Homo sapiens
MWGRLLLWPLVLGFSLSGGTQTPSVYDESGSTGGGDDSTPSILPAPRGYPGQVCANDSDTLELPDSSRALLLGWVPTRLVPALYGLVLVVGLPANGLALWVLATQAPRLPSTMLLMNLATADLLLALALPPRIAYHLRGQRWPFGEAACRLATAALYGHMYGSVLLLAAVSLDRYLALVHPLRARALRGRRLALGLCMAAWLMAAALALPLTLQRQTFRLARSDRVLCHDALPLDAQASHWQPAFTCLALLGCFLPLLAMLLCYGATLHTLAASGRRYGHALRLTAVVLASAVAFFVPSNLLLLLHYSDPSPSAWGNLYGAYVPSLALSTLNSCVDPFIYYYVSAEFRDKVRAGLFQRSPGDTVASKASAEGGSRGMGTHSSLLQ

SEQ ID NO: 114 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLLIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 115 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 116 - protein, 1186 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 117 - protein, 422 aa, Homo sapiens
MDVLSPGQGNNTTSPPAPFETGGNTTGISDVTVSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSDPDACTISKDHGYTIYSTFGAFYIPLLLMLVLYGRIFRAARFRIRKTVKKVEKTGADTRHGASPAPQPKKSVNGESGSRNWRLGVESKAGGALCANGAVRQGDDGAALEVIEVHRVGNSKEHLPLPSEAGPTPCAPASFERKNERNAEAKRKMALARERKTVKTLGIIMGTFILCWLPFFIVALVLPFCESSCHMPTLLGAIINWLGYSNSLLNPVIYAYFNKDFQNAFKKIIKCKFCRQ

SEQ ID NO: 118 - protein, 129 aa, Homo sapiens [sequence omitted]

SEQ ID NO: 119 - protein, 113 aa, Homo sapiens
MGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 120 - protein, 726 aa, Homo sapiens
VTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 121 - protein, 191 aa, Homo sapiens [sequence omitted]

SEQ ID NO: 122 - protein, 156 aa, Homo sapiens
AYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 123 - protein, 735 aa, Homo sapiens
EVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 124 - protein, 509 aa, Homo sapiens
MKVKGTRRNYQHLWRWGTLLLGMLMICSATEKLWVTVYYGVPVWKEATTTLFCASDARAYDTEVHNVWATHACVPTDPNPQEVVLGNVTENFNMWKNNMVEQMQEDIISLWDQSLKPCVKLTPLCVTLNCTDLGKATNTNSSNWKEEIKGEIKNCSFNITTSIRDKIQKENALFRNLDVVPIDNASTTTNYTNYRLIHCNRSVITQACPKVSFEPIPIHYCTPAGFAILKCNNKTFNGKGPCTNVSTVQCTHGIRPIVSTQLLLNGSLAEEEVVIRSDNFTNNAKTIIVQLNESVAINCTRPNNNTRKSIYIGPGRAFHTTGRIIGDIRKAHCNISRAQWNNTLEQIVKKLREQFGNNKTIVFNQSSGGDPEIVMHSFNCRGEFFYCNTTQLFNNTWRLNHTEGTKGNDTIILPCRIKQIINMWQEVGKAMYAPPIGGQISCSSNITGLLLTRDGGTNVTNDTEVFRPGGGDMRDNWRSELYKYKVIKIEPLGIAPTKAKRRVVQREKR

SEQ ID NO: 125 - protein, 101 aa, Homo sapiens
SWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQ

SEQ ID NO: 126 - protein, 459 aa, Homo sapiens
EICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCE

SEQ ID NO: 127 - protein, 146 aa, Homo sapiens
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 128 - protein, 327 aa, Homo sapiens
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPQQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 129 - protein, 433 aa, Homo sapiens
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 130 - protein, 1145 aa, Homo sapiens
YNLDVRGARSFSPPRAGRHFGYRVLQVGNGVIVGAPGEGNSTGSLYQCQSGTGHCLPVTLRGSNYTSKYLGMTLATDPTDGSILACDPGLSRTCDQNTYLSGLCYLFRQNLQGPMLQGRPGFQECIKGNVDLVFLFDGSMSLQPDEFQKILDFMKDVMKKLSNTSYQFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLYPQFQGRLVANLTYTLQLDGHRTRRRGLFPGGRHELRRNIAVTTSMSCTDFSFHFPVCVQDLISPINVSLNFSLWEEEGTPRDQRAQGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 131 - protein, 660 aa, Homo sapiens
MEALMARGALTGPLRALCLLGCLLSHAAAAPSPIIKFPGDVAPKTDKELAVQYLNTFYGCPKESCNLFVLKDTLKKMQKFFGLPQTGDLDQNTIETMRKPRCGNPDVANYNFFPRKPKWDKNQITYRIIGYTPDLDPETVDDAFARAFQVWSDVTPLRFSRIHDGEADIMINFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 132 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 133 - protein, 115 aa, Homo sapiens
IPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 134 - protein, 185 aa, Homo sapiens
APVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 135 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 136 - protein, 472 aa, Homo sapiens
EMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 137 - protein, 143 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 138 - protein, 143 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYRE

SEQ ID NO: 139 - protein, 440 aa, Homo sapiens
EMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK

SEQ ID NO: 140 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 141 - protein, 762 aa, Homo sapiens
GPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDREPYMTENTCNRYCRDEIESVKELKDTGKDAVNCTYKNEDDCVVRFQYYEDSSGKSILYVVEEPECPKGPDILVVLLSVMGAILLIGLAALLIWKLLITIHDRKEFAKFEEERARAKWDTANNPLYKEATSTFTNITYRGT

SEQ ID NO: 142 - protein, 505 aa, Homo sapiens
QTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTREVTVNVLSPRYEIVIITVVAAAVIMGTAGLSTYLYNRQRKIKKYRLQQAQKGTPMKPNTQATPP

SEQ ID NO: 143 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 144 - protein, 187 aa, Homo sapiens
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 145 - protein, 544 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENAESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 146 - protein, 358 aa, Homo sapiens
CEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVLPPSSTKPPALSHSVSTSSTTKSPASSASGPRPTYKPPVSNYPGYPKPEEGILDSLDVWVIAVIVIAIVVGVAVICVVPYRYLQRRKKKGTYLTDETHREVKFTSL

SEQ ID NO: 147 - protein, 1148 aa, Homo sapiens
LPEAKIFSGPSSEQFGYAVQQFINPKGNWLLVGSPWSGFPENRMGDVYKCPVDLSTATCEKLNLQTSTSIPNVTEMKTNMSLGLILTRNMGTGGFLTCGPLWAQQCGNQYYTTGVCSDISPDFQLSASFSPATQPCPSLIDVVVVCDESNSIYPWDAVKNFLEKFVQGLDIGPTKTQVGLIQYANNPRVVFNLNTYKTKEEMIVATSQTSQYGGDLTNTFGAIQYARKYAYSAASGGRRSATKVMVVVTDGESHDGSMLKAVIDQCNHDNILRFGIAVLGYLNRNALDTKNLIKEIKAIASIPTERYFFNVSDEAALLEKAGTLGEQIFSIEGTVQGGDNFQMEMSQVGFSADYSSQNDILMLGAVGAFGWSGTIVQKTSHGHLIFPKQAFDQILQDRNHSSYLGYSVAAISTGESTHFVAGAPRANYTGQIVLYSVNENGNITVIQAHRGDQIGSYFGSVLCSVDVDKDTITDVLLVGAPMYMSDLKKEEGRVYLFTIKKGILGQHQFLEGPEGIENTRFGSAIAALSDINMDGFNDVIVGSPLENQNSGAVYIYNGHQGTIRTKYSQKILGSDGAFRSHLQYFGRSLDGYGDLNGDSITDVSIGAFGQVVQLWSQSIADVAIEASFTPEKITLVNKNAQIILKLCFSAKFRPTKQNNQVAIVYNITLDADGFSSRVTSRGLFKENNERCLQKNMVVNQAQSCPEHIIYIQEPSDVVNSLDLRVDISLENPGTSPALEAYSETAKVFSIPFHKDCGEDGLCISDLVLDVRQIPAAQEQPFIVSNQNKRLTFSVTLKNKRESAYNTGIVVDFSENLFFASFSLPVDGTEVTCQVAASQKSVACDVGYPALKREQQVTFTINFDFNLQNLQNQASLSFQALSESQEENKADNLVNLKIPLLYDAEIHLTRSTNINFYEISSDGNVPSIVHSFEDVGPKFIFSLKVTTGSVPVSMATVIIHIPQYTKEKNPLMYLTGVQTDKAGDISCNADINPLKIGQTSSSVSFKSENFRHTKELNCRTASCSNVTCWLKDVHMKGEYFVNVTTRIWNGTFASSTFQTVQLTAAAEINTYNPEIYVIEDNTVTIPLMIMKPDEKAEVPTGVIIGSIIAGILLLLALVAILWKLGFFKRKYEKMTKNPDEIDETTELSS

SEQ ID NO: 148 - protein, 133 aa, Homo sapiens
APMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 149 - protein, 622 aa, Homo sapiens
MAHVRGLQLPGCLALAALCSLVHSQHVFLAPQQARSLLQRVRRANTFLEEVRKGNLERECVEETCSYEEAFEALESSTATDVFWAKYTACETARTPRDKLAACLEGNCAEGLGTNYRGHVNITRSGIECQLWRSRYPHKPEINSTTHPGADLQENFCRNPDSSTTGPWCYTTDPTVRRQECSIPVCGQDQVTVAMTPRSEGSSVNLSPPLEQCVPDRGQQYQGRLAVTTHGLPCLAWASAQAKALSKHQDFNSAVQLVENFCRNPDGDEEGVWCYVAGKPGDFGYCDLNYCEEAVEEETGDGLDEDSDRAIEGRTATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

(19) United States
(12) Patent Application Publication
(10) Pub. No.: US 2005/0002897 A1
(43) Pub. Date: Jan. 6, 2005

(54) Biological entities and the pharmaceutical or diagnostic use thereof

(76) Inventors: Ulrich Haupts, Koln (DE); Andre Koltermann, Koln (DE); Andreas Scheidig, Koln (DE); Christian Votsmeier, Koln (DE); Ulrich Kettling, Koln (DE)

Correspondence Address: NEEDLE & ROSENBERG, P.C., SUITE 1000, 999 PEACHTREE STREET, ATLANTA, GA, 30309-3915, US

(21) Appl. No.: 10/872,198

(22) Filed: Jun. 18, 2004

Related U.S. Application Data
(60) Provisional application No. 60/524,960, filed on Nov. 25, 2003.
(60) Provisional application No. 60/543,518, filed on Feb. 11, 2004.

(30) Foreign Application Priority Data
Jun. 18, 2003 (EP) 03013819
Nov. 10, 2003 (EP) 03025851
Nov. 11, 2003 (EP) 03025871
Feb. 11, 2004 (EP) 04003058

(51) Int. Cl. C12N 9/64; A61K 38/48; A61K 38/21

(52) U.S. Cl. 424/85.1; 424/85.2; 424/85.4; 424/94.63

(57) ABSTRACT

The present invention provides method for the treatment of a disease by applying a medicament comprising a protease with a defined specificity is capable to hydrolyze specific peptide bonds within a target substrate related to such disease. The proteases with such a defined specificity can further be used for related therapeutic or diagnostic purposes.

This application claims the priority benefit of European Application No. 03013819, filed Jun. 18, 2003; European Application No. 03025851, filed Nov. 10, 2003; European Application No. 03025871, filed Nov. 11, 2003; U.S. Provisional Application No. 60/524,960, filed Nov. 25, 2003; European Application No. 04003058, filed Feb. 11, 2004; and U.S. Provisional Application No. 60/543,518, filed Feb. 11, 2004, which applications are incorporated herein fully by this reference.

The present invention provides methods for the treatment of a disease by applying a medicament comprising a protease with a defined specificity is capable to hydrolyze specific peptide bonds within a target substrate related to such disease. The proteases with such a defined specificity can further be used for related therapeutic or diagnostic purposes.

### BACKGROUND

Academic and industrial research continuously searches for functional proteins to be used as therapeutic, research, diagnostic, nutritional, personal care or industrial agents. Today, such functional proteins can be classified mainly into two categories: natural proteins and engineered proteins. Natural proteins, on the one hand, are discovered from nature, e.g. by screening natural isolates or by sequencing genomes from diverse species. Engineered proteins, on the other hand, are typically based on known proteins and are altered in order to acquire modified functionalities. The present invention discloses engineered proteins with novel functions as compared to the starting components. Such proteins are called NBEs (New Biologic Entities). The NBEs disclosed in the present invention are engineered enzymes with novel substrate specificities or fusion proteins of such engineered enzymes with other functional components.

Specificity is an essential element of enzyme function. A cell consists of thousands of different, highly reactive catalysts. Yet the cell is able to maintain a coordinated metabolism and a highly organized three-dimensional structure. This is due in part to the specificity of enzymes, i.e. the selective conversion of their respective substrates. Specificity is a qualitative and a quantitative property: the specificity of a particular enzyme can vary widely, ranging from just one particular type of target molecules to all molecular types with certain chemical substructures. In nature, the specificity of an organism's enzymes has been evolved to the particular needs of the organism. Arbitrary specificities with high value for therapeutic, research, diagnostic, nutritional or industrial applications are unlikely to be found in any organism's enzymatic repertoire due to the large space of possible specificities. The only realistic way of obtaining such specificities is their generation de novo.

When comparing enzymes with binders, a paradigm of specificity is given by antibodies recognizing individual epitopes as small distinct structures within large molecules. The naturally occurring vast range of antibody specificities is attributed to the diversity generated by the immune system combined with natural selection. Several mechanisms contribute to the vast repertoire of antibody specificity and occur at different stages of immune response generation and antibody maturation (Janeway, C et al. (1999) Immunobiology, Elsevier Science Ltd., Garland Publishing, New York). Specifically, antibodies contain complementarity determining regions (CDRs) which interact with the antigen in a highly specific manner and allow discrimination even between very similar epitopes. The light as well as the heavy chain of the antibody each contribute three CDRs to the binding domain. Nature uses recombination of various gene segments combined with further mutagenesis in the generation of CDRs. As a result, the sequences of the six CDR loops are highly variable in composition and length and this forms the basis for the diversity of binding specificities in antibodies. A similar principle for the generation of a diversity of catalytic specificities is not known from nature.

Catalysis, i.e. the increase of the rate of a specific chemical reaction, is besides binding the most important protein function. Catalytic proteins, i.e. enzymes, are classified according to the chemical reaction they catalyze.

Transferases are enzymes transferring a group, for example, the methyl group or a glycosyl group, from one compound (generally regarded as donor) to another compound (generally regarded as acceptor). For example, glycosyltransferases (EC 2.4) transfer glycosyl residues from a donor to an acceptor molecule. Some of the glycosyltransferases also catalyze hydrolysis, which can be regarded as transfer of a glycosyl group from the donor to water. The subclass is further subdivided into hexosyltransferases (EC 2.4.1), pentosyltransferases (EC 2.4.2) and those transferring other glycosyl groups (EC 2.4.99, Nomenclature Committee of the International Union of Biochemistry and Molecular Biology (NC-IUBMB)).

Oxidoreductases catalyze oxido-reductions. The substrate that is oxidized is regarded as hydrogen or electron donor. Oxidoreductases are classified as dehydrogenases, oxidases, mono- and dioxygenases. Dehydrogenases transfer hydrogen from a hydrogen donor to a hydrogen acceptor molecule. Oxidases react with molecular oxygen as hydrogen acceptor and produce oxidized products as well as either hydrogen peroxide or water. Monooxygenases transfer one oxygen atom from molecular oxygen to the substrate and one is reduced to water. In contrast, dioxygenases catalyze the insert of both oxygen atoms from molecular oxygen into the substrate.

Lyases calalyze elimination reactions and thereby generate double bonds or, in the reverse direction, catalyze the additions at double bonds. Isomerases catalyze intramolecular rearrangements. Ligases catalyze the formation of chemical bonds at the expense of ATP consumption.

Finally, hydrolases are enzymes that catalyze the hydrolysis of chemical bonds like C—O or C—N. The E.C. classification for these enzymes generally classifies them by the nature of the bond hydrolysed and by the nature of the substrate. Hydrolases such as lipases and proteases play an important role in nature as well in technical applications of biocatalysts. Proteases hydrolyse a peptide bond within the context of an oligo- or polypeptide. Depending on the catalytic mechanism proteases are grouped into aspartic, serin, cysteine, metallo- and threonine proteases (Handbook of proteolytic enzymes. (1998) Eds: Barret, A; Rawling, N.; Woessner, J.; Academic Press, London). This classification is based on the amino acid side chains that are responsible for catalysis and which are typically presented in the active site in very similar orientation to each other. The scissile bond of the substrate is brought into register with the catalytic residues due to specific interactions between the amino acid side chains of the substrate and complementary regions of the protease (Perona, J. & Craik, C (1995) Protein Science, 4, 337-360). The residues on the N- and C-terminal side of the scissile bond are usually called P₁, P₂, P₃ etc and P₁′, P₂′, P₃′ and the binding pockets complementary to the substrate S₁, S₂, S₃ and S₁′, S₂′, S₃′, respectively (nomenclature according to Schlechter & Berger, Biochem. Biophys. Res. Commun. 27 (1967) 157-162). The selectivity of proteases can vary widely from being virtually nonselective—e.g. the Subtilisins—over a strict preference at the P₁ position—e.g. Trypsin selectively cutting on the C-terminal side of arginine or lysine residues—to highly specific proteases—e.g. human tissue-type plasminogen activator (t-PA) cleaving at the C-terminal side of the arginine in the sequence CPGRVVG (Ding, L et al. (1995) Proc. Natl. Acad. Sci. USA 92, 7627-7631; Coombs, G et al. (1996) J. Biol. Chem. 271, 4461-4467).

The specificity of proteases, i.e. their ability to recognize and hydrolyze preferentially certain peptide substrates, can be expressed qualitatively and quantitatively. Qualitative specificity refers to the kind of amino acid residues that are accepted by a protease at certain positions of the peptide substrate. For example, trypsin and t-PA are related with respect to their qualitative specificity, since both of them require at the P₁ position an arginine or a similar residue. On the other hand, quantitative specificity refers to the relative number of peptide substrates that are accepted as substrates by the protease, or more precisely, to the relative k_{cat}/k_{M} ratios of the protease for the different peptides that are accepted by the protease. Proteases that accept only a small portion of all possible peptides have a high specificity, whereas the specificity of proteases that, as an extreme, cleave any peptide substrate would theoretically be zero.

Comparison of the primary, secondary as well as the tertiary structure of proteases (Fersht, A., Enzyme Structure and Mechanism, W. H. Freeman and Company, New York, 1995) allows identification of classes showing a high degree of conservation (Rawlings, N. D. & Barrett, A. J. (1997) In: Proteolysis in Cell Functions Eds. Hopsu-Havu, V. K.; Järvinen, M.; Kirschke, H, pp. 13-21, IOS Press, Amsterdam). A widely accepted scheme for protease classification has been proposed by Rawlings & Barrett (Handbook of proteolytic enzymes. (1998) Eds: Barret, A; Rawling, N.; Woessner, J.; Academic Press, London). For example, the serine proteases family can be subdivided into structural classes with chymotrypsin (class S1), subtilisin (class S8) and carboxypeptidase (class SC) folds, each of which includes nonspecific as well as specific proteases (Rawlings, N. D. & Barrett, A. J. (1994) Methods Enzymol. 244, 19-61). This applies to other protease families analogously. An additional distinction can be made according to the relative location of the cleaved bond in the substrate. Carboxy- and aminopeptidases cleave amino acids from the C- and N-terminus, respectively, while endopeptidases cut anywhere along the oligopeptide.

Many applications would be conceivable if enzymes with a basically unlimited spectrum of specificities were available. However, the use of such enzymes with high, low or any defined specificity is currently limited to those which can be isolated from natural sources. The field of application for these enzymes varies from therapeutic, research, diagnostic, nutritional to personal care and industrial purposes.

Enzyme additives in detergents have come to constitute nearly a third of the whole industrial enzyme market. Detergent enzymes include proteinases for removing organic stains, lipases for removing greasy stains, amylases for removing residues of starchy foods and cellulases for restoring of smooth surface of the fiber. The best-known detergent enzyme is probably the nonspecific proteinase subtilisin, isolated from various Bacillus species.

Starch enzymes, such as amylases, occupy the majority of those used in food processing. While starch enzymes include products that are important for textile desizing, alcohol fermentation, paper and pulp processing, and laundry detergent additives, the largest application is for the production of high fructose corn syrup. The production of corn syrup from starch by means of industrial enzymes was a successful alternative to acid hydrolysis.

Apart from starch processing, enzymes are used for an increasing range of applications in food. Enzymes in food can improve texture, appearance and nutritional value or may generate desirable flavours and aromas. Currently used food enzymes in bakery are amylase, amyloglycosidases, pentosanases for breakdown of pentosan and reduced gluten production or glucose oxidases to increase the stability of dough. Common enzymes for dairy are rennet (protease) as coagulant in cheese production, lactase for hydrolysis of lactose, protease for hydrolysis of whey proteins or catalase for the removel of hydrogen peroxides. Enzymes used in brewing process are the above named amylases, but also cellulases or proteases to clarify the beer from suspended proteins. In wines and fruit juices, cloudiness is more commenly caused by starch and pectins so that amylases and pectinases increase yield and clarification. Papain and other proteinases are used for meat tenderizing.

Enzymes have also been developed to aid animals in the digestion of feed. In the western hemisphere, corn is a major source of food for cattle, swine, and poultry. In order to improve the bioavailability of phosphate from corn, phytase is commonly added (Wyss, M. et al., Biochemical characterization of fungal phytases (myo-inositol hexakisphosphate phosphohydrolases); Catalytic properties. Applied & Environmental Microbiology 65, 367-373 (1999)). Moreover, phytate hydrolysis has been shown to bring about improvements in digestibility of protein and absorption of minerals such as calcium (Bedford, M. R. & Schulze, H., Exogenous Enzymes for Pigs and Poultry [Review]. Nutrition Research Reviews 11, 91-114 (1998)). Another major feed enzyme is xylanase. This enzyme is particularly useful as a supplement for feeding stuff comprising more than about 10% of wheat barley or rye, because of their relatively high soluble fiber content. Xylanases cause two important actions: reduction of viscosity of the intestinal contents by hydrolyzing the gel-like high molecular weight arabinoxylans in feed (Murphy, T et al., Effect of range of new xylanases on in vitro viscosity and on performance of broiler diets. British Pultry Science 44, S16-S18 (2003)) and break down of polymers in cell wallswhich improve the bioavailability of protein and starch.

Biotech research and development laboratories routinely use special enzymes in small quantities along with many other reagents. These enzymes create a significant market for various enzymes. Enzymes like alkaline phosphatase, horseradish peroxidase and luciferase are only some examples. Thermostable DNA polymerases like Taq polymerase or restriction endonucleases revolutionized laboratory work.

The use of enzymes in the diagnosis of disease is another important benefit derived from the intensive research in biochemistry. Within the recent past few years that interest in diagnostic enzymology has increased and there are still large areas of medical research in which the diagnostic potential of enzyme reactions has not been explored at all. Common enzymes used for clinical diagnosis are acid phosphatase, alanine aminotransferase, alkaline phosphatase, amylase, angiotensin converting enzymes, aspartate aminotransferase, cholinesterase, creatinine kinase, gamma glutamyltransferase, lactate dehydrogenaseor rennin.

Therapeutic enzymes are a particular class of drugs, categorized by the FDA as biologicals, with a lot of advantages compared to other, especially non-biological pharmaceuticals. Examples for successful therapeutic enzymes are human clotting factors like factor VIII and factor IX for human treatment. In addition, digestive enzymes are used for various deficiencies in human digestive processes. Other examples are t-PA and streptokinase for the treatment of cardiovascular disease, beta-glucocerebrosidase for the treatment of Type I Gaucher disease, L-asparaginase for the the treatment of acute lymphoblastic leukemia and DNAse for the treatment of cystic fibrosis. An important issue in the application of proteins as therapeutics is their potential immunogenicity. To reduce this risk, one would prefer enzymes of human origin, which narrows down the set of available enzymes. The provision of designed enzymes, preferably of human origin, with novel, tailor-made specificities would allow the specific modification of target substrates at will, while minimizing the risk of immunogenicity. A further advantage of highly specific enzymes as therapeutics would be their lower risk of side effects. Due to the limited possibility of specific interactions between a small molecule and a protein, binding to non-target proteins and therefore side effects are quite common and often cause termination of an otherwise promising lead compound. Specific enzymes, on the other hand, provide many more contact sites and mechanisms for substrate discrimination and therefore enable a higher specificity and thereby less side activities.

Proteases represent an important class of therapeutic agents (Drugs of today, 33, 641-648 (1997)). However, currently the therapeutic protease is usually a substitute for insufficient acitivity of the body's own proteases. For example, factor VII can be administered in certain cases of coagulation deficiencies of bleeders or during surgery (Heuer L.; Blumenberg D. (2002) Anaesthesist 51:388). Tissue-type plasminogen activator (t-PA) is applied in acute cardiac infarction, initializing the dissolution of fibrin clots through specific cleavage and activation of plasminogen (Verstraete, M. et al. (1995) Drugs, 50, 29-41). So far a protease with taylor-made specificity is generated to provide a therapeutic agent that specifically activates or inactivates a disease related target protein.

Monoclonal antibodies represent another important biological class of substances with therapeutic capabilities. One of the main antibody targets are tumor necrosis factors (TNFs) which belong to the family of cytokines. TNFs play a major role in the inflammation process. As homotrimers they could bind to receptors of nearly every cell. They activate a multiplicity of cellular genes, multiple signal transduction mechanisms, kinases and transcription factors. The most important TNFs are TNF-alpha and TNF-beta. TNF-alpha is produced by macrophages, monocytes and other cells. TNF-alpha is an inflammation mediator. Therefore, research of the last decade has been focused on TNF-alpha inhibitors like monoclonal antibodies as possible therapeutics for different therapeutic indications like Rheumatoid Arthritis, Crohn's disease or Psoriasis (Hamilton et al. (2000) Expert Opin Pharmacother, 1 (5): 1041-1052). One of the major disadvantages of monoclonal antibodies are their high costs, so that new biological alternatives are of great importance.

There are a lot of examples for engineered enzymes in literature. Fulani et al. (Fulani F. et al. (2003) Protein Engineering 16, 515-519) describe a rhodanase (thiosulfat:cyanide sulfurtransferase) from Azotobacter vinelandii which has a catalytic domain structurally related to catalytic subunit of Cdc25 phosphatase enzymes. The difference in catalytic mechanism depends on the different size of the active site. Both rhodanase and phosphatase are highly specific on different substrates (sulfate vs. phosphate). The catalytic mechanism of the rhodanase could be shifted towards serine/threonine phosphatase by single-residue insertion. Therefore, Fulani et al. give a single example for the change of a catalytic mechanism by structural comparison and sequence alignment of naturally known enzymes from different enzyme classes but lack an indication of how to generate a user-definable substrate specificity while keeping the same catalytic mechanism.

The thioredoxin reductase described by Briggs et al. (WO 02/090300 A2) has an altered cofactor specificity which preferably binds NADPH compared to NADH. Thus, both enzymes, the starting point as well as the resulting engineered enzyme are highly specific towards different substrates. The methods to achieve such an altered substrate specificity are either computational processing methods or sequence alignments of related proteins to define variable and conserved residues. They all have in common that they are based on the comparison of structures and sequences of proteins with known specificities followed by the transfer of the same to another backbone.

There are other examples of specificity-engineered enzymes and, in particular, of proteases which have been published in the literature. None of these examples, however, provides a means for generating novel specificites compared to the specificity of the starting material used within the described methods. The methods range from structure-directed single point mutations (Kurth, T. et al. (1998) Biochemistry 37, 11434-11440; Ballinger, M et al. (1996) Biochemistry, 35:13579-13585), exchange of surface loops between two specific proteases (Horrevoets et al. (1993) J. Biol. Chem. 268, 779-782), to random mutagenesis either regio-selectively or across the whole gene combined with in-vitro or in-vivo selection (Sices, H. & Kristie, T. (1998) Proc. Natl. Acad. Sci. USA, 95, 2828-2833).

The rational design of protease specificity is limited to very few examples. This approach is severely limited by the insufficient understanding of the complexities that govern folding and dynamics as well as structure-function relationships in proteins (Corey, M. J. & Corey, E. (1996) Proc. Natl. Acad. Sci. USA, 93:11428-11434). It is therefore difficult to alter the primary amino acid sequence of a protease in order to change its activity or specificity in a predictive way. In a successful example, Kurth et al. engineered trypsin to show a preference for a dibasic motive (Kurth, T. et al. (1998) Biochemistry, 37:11434-11440). In another example, Hedstrom et al. converted the S₁ substrate specificity of trypsin to that of chymotrypsin (Hedstrom, L. et al. (1992) Science, 255:1249-1253). This is an example where a known property was transferred from one backbone to another.

Ballinger et al. (WO 96/27671) describe subtilisin variants with combination mutations (N62D/G166D, and optionally Y104D) having a shift of substrate specificity towards peptide or polypeptide substrates with basic amino acids at the P1, P2 and P4 positions of the substrate. Suitable substrates of the variant subtilisin were revealed by sorting a library of phage particles (substrate phage) containing five contiguous randomized residues. These subtilisin variants are useful for cleaving fusion proteins with basic substrate linkers and processing hormones or other proteins (in vitro or in vivo) that contain basic cleavage sites.

The problems associated with rational redesign of enzymes can partially be overcome by directed evolution (as disclosed in PCT/EP03/04864). These studies can be classified by their expression and selection systems. Genetic selection means to produce inside an organism an enzyme, e.g. a protease, which is able to cleave a precursor protein which in turn results in an alteration of the growth behavior of the producing organism. From a population of organisms with different proteases those can be selected which have an altered growth behavior. This principle was for example reported by Davis et al. (U.S. Pat. No. 5,258,289, WO 96/21009). The production of a phage system is dependent on the cleavage of a phage protein which only can be activated in the presence of a proteolytic enzyme which is able to cleave the phage protein. Other approaches use a reporter system which allows a selection by screening instead of a genetic selection, but also cannot overcome the intrinsic insufficiency of the intracellular characterization of enzymes.

Systems to generate enzymes with altered sequence specificities with self-secreting enzymes are also reported. Duff et al. (WO 98/11237) describe an expression system for a self-secreting protease. An essential element of the experimental design is that the catalytic reaction acts on the protease itself by an autoproteolytic processing of the membrane-bound precursor molecule to release the matured protease from the cellular membrane into the extracellular environment. Therefore, a fusion protein must be constructed where the target peptide sequence replaces the natural cleavage site for autoproteolysis. Limitations of such a system are that positively identified proteases will have the ability to cleave a certain amino acid sequence but they also may cleave many other peptide sequences. Therefore, high substrate specificity cannot be achieved. Additionally, such a system is not able to control that selected proteases cleave at a specific position in a defined amino acid sequence and it does not allow a precise characterization of the kinetic constants of the selected proteases (k_{cat}, K_{M}).

A method has been described that aims at the generation of new catalytic activities and specificities within the α/β-barrel proteins (WO 01/42432; Fersht et al, Methods of producing novel enzymes; Altamirano et al. (2000) Nature 403, 617-622). The α/β-barrel proteins comprise a large superfamily of proteins accounting for a large fraction of all known enzymes. The structure of the proteins is made from α/β-barrel surrounded by α-helices. The loops connecting B-strands and helices comprise the so-called lid-structure including the acitve site residues. The method is based on the classification of α/β-barrel proteins into two classes based on the catalytic lid structure. An extensive comparison of α/β-barrel protein structures led the authors to the conclusion that the substrate binding and specificity is primarily defined by the barrel structure while the specificity of the chemical reaction resides within the loops. It is suggested that barrels and lid structures from different enzymes can be combined to generate new enzymatic activities and to provide a starting point to fine tune the properties by targeted or randomized mutagenesis and selection. The method does not provide for the generation of user-defined specificity.

In summary, it is clear that there are many possible applications in the fields of therapeutics, research and diagnostics, industrial enzymes, food and feed processing, cosmetics and other areas that would become possible by the availability of enzymes with a novel substrate specificity. However, only a limited number of specific enzymes has been identified from natural sources so far. Methods of rational design to modify, alter, convert or transfer sequence specificity as well as random approaches described above did not enable the generation of a novel and user-definablespecificity that was not present in the employed starting material.

Therefore, none of the currently available methods can provide enzymes with a novel and user-defined sequence specificity. In contrast, the current invention provides such enzymes as well as methods for generating them.

### SUMMARY OF THE INVENTION

The objective of the present invention is to provide a method for the treatment of a disease by applying a medicament comprising a protease. Further the present invention provides engineered proteins with novel functions that do not exist in the components used for the engineering of such proteins. In particular, the invention provides enzymes with user-definable specificities. User-definable specificity means that enzymes are provided with specificities that do not exist in the components used for the engineering of such enzymes. The specificities can be chosen by the user so that one or more intended target substrates are preferentially recognised and converted by the enzymes. Furthermore, the invention provides enzymes that possess essentially identical sequences to human proteins but have different specificities. In a particular embodiment, the invention provides proteases with user-definable specificities.

Furthermore, the present invention is directed to engineered enzymes which are fused to one or more further functional components. These further components can be proteinacious components which preferably have binding properties and are of the group consisting of substrate binding domains, antibodies, receptors or fragments thereof. Furthermore, these further components can be further functional components, preferably being selected from the group consisting of polyethylenglycols, carbohydrates, lipids, fatty acids, nucleic acids, metals, metal chelates, and fragments or derivatives thereof. The resulting fusion proteins are understood as enzymes with user-definable specificities within the present invention.

Besides, the invention is directed to the application of such enzymes with novel, user-definable specificities for therapeutic, research, diagnostic, nutritional, personal care or industrial purposes. Moreover, the invention is directed to a method for generating engineered enzymes with user-definable specificities. In particular, the invention is directed to generate enzymes that possess essentially identical sequences to human enzymes but have different specificities.

This problem has been solved by the embodiments of the invention specified in the description below and in the claims. The present invention is thus directed to

(1) the use of a protease with defined specificity for a target substrate for preparing a medicament for the treatment of a specific disease related to said target substrate,

(2) an engineered enzyme with defined specificity characterized by the combination of the following components,:

(a) a protein scaffold which catalyzes at least one chemical reaction on at least one substrate, and

(b) one or more specificity determining regions (SDRs) located at sites in the protein scaffold that enable the resulting engineered protein to discriminate between at least one target substrate and one or more different substrates, and wherein the SDRs are essentially synthetic peptide sequences;

(3) the use of an engineered enzyme as defined in (2) above for therapeutic, research, diagnostic, nutritional, personal care or industrial purposes, preferably for the use as defined in (1) above;

(4) a method for generating engineered enzymes as defined in (2) above having specificities towards target substrates, such specificities not being present in the individual starting components, comprising at least the following steps:

(a) providing a protein scaffold which catalyzes at least one chemical reaction on at least one substrate,

(b) generating a library of engineered enzymes by combining the protein scaffold from step (a) with fully or partially random peptide sequences at sites in the protein scaffold that enable the resulting engineered enzyme to discriminate between at least one target substrate and one or more different substrates, and

(c) selecting out of the library of engineered enzymes generated in step (b) one or more enzymes that have specificities towards at least one target substrate;

(5) a fusion protein which is comprised of at least one engineered enzyme as defined in (2) above and at least one further component, preferably the at least one further component having binding properties and more preferably being selected from the group consisting of antiboides, binding domains, receptors, and fragments thereof;

(6) a composition or pharmaceutical composition comprising one or more engineered enzymes as defined in (2) above or a fusion protein as defined in (5) above, said pharmaceutical composition may optionally comprise an acceptable carrier, excipient and/or auxiliary agent;

(7) a DNA encoding the engineered enzyme as defined in (2) above;

(8) a vector comprising the DNA as defined in (7) above;

(9) a host cell or transgenic organism being transformed/transfected with a vector as defined in (8) above and/or containing the DNA as defined in (7) above; and

(10) a method for producing the engineered enzyme of (2) above comprising culturing a cell or organism as defined in (8) above and isolating the enzyme from the culture broth.

### BRIEF DESCRIPTION OF THE FIGURES

The following figures are provided in order to explain further the present invention in supplement to the detailed description:

FIG. 1 illustrates the three-dimensional structure of human trypsin I with the active site residues shown in “ball-and-stick” representation and with the marked regions indicating potential SDR insertion sites.

FIG. 2 shows the alignment of the primary amino acid sequence of three members of the serine protease class S1 family: human trypsin I, human alpha-thrombin and human enteropeptidase (see also SEQ ID NOs: 1, 5 and 6).

FIG. 3 illustrates the three-dimensional structure of subtilisin with the active site residues being shown in “ball-and-stick” representation and with the numbered regions indicating potential SDR insertion sites.

FIG. 4 shows the alignment of the primary amino acid sequences of four members of the serine protease class S8 family: subtilisin E, furin, PC1 and PC5 (see also SEQ ID NOs: 7-10).

FIG. 5 illustrates the three-dimensional structure of pepsin with the active site residues being shown in “ball-and-stick” representation and with the numbered regions indicating potential SDR insertion sites.

FIG. 6 shows the alignment of the primary amino acid sequences of three members of the Al aspartic acid protease family: pepsin, β-secretase and cathepsin D (see also SEQ ID NOs: 11-13).

FIG. 7: illustrates the three-dimensional structure of caspase 7 with the active site residues being shown in “ball-and-stick” representation and with the numbered regions indicating potential SDR insertion sites.

FIG. 8: shows the primary amino acid sequence of caspase 7 as a member of the cysteine protease class C14 family (see also SEQ ID NO: 14).

FIG. 9 depicts schematically the third aspect of the invention.

FIG. 10 shows a Western blot analysis of a culture supernatant of cells expressing variants of human trypsin I with SDR1 and SDR2, compared to negative controls.

FIG. 11 shows the time course of the proteolytic cleavage of a target substrate by human trypsin I.

FIG. 12 shows the relative activities of three variants of inventive engineered proteolytic enzymes in comparison with human trypsin I on two different peptide substrates.

FIG. 13 shows the relative specificities of human trypsin I and variants of inventive engineered proteolytic enzymes with one or two SDRs, respectively.

FIG. 14: shows the relative specificities of human trypsin I and of variants of inventive engineered proteolytic enzymes being specific for human TNF-alpha with this scaffold on peptides with a target sequence of human TNF-alpha.

FIG. 15: shows the reduction of cytotoxicity induced by TNF-alpha when incubating the TNF-alpha with concentrated supernatant from cultures expressing the inventive engineered proteolytic enzymes being specific for human TNF-alpha.

FIG. 16: shows the reduction of cytotoxicity induced by TNF-alpha when incubating the TNF-alpha with purified inventive engineered proteolytic enzyme being specific for human TNF-alpha.

FIG. 17: compares the activity of inventive engineered proteolytic enzymes being specific for human TNF-alpha with the activity of human trypsin I on two protein substrates: (a) human TNF-alpha; (b) mixture of human serum proteins.

FIG. 18: showes the specific activity of an inventive engineered proteolytic enzyme with specificity for human VEGF.

### DEFINITIONS

In the framework of the present invention the following terms and definitions are used.

The term “protease” means any protein molecule that is capable of hydrolysing peptide bonds. This includes naturally-occurring or artificial proteolytic enzymes, as well as variants thereof obtained by site-directed or random mutagenesis or any other protein engineering method, any active fragment of a proteolytic enzyme, or any molecular complex or fusion protein comprising one of the aforementioned proteins. A “chimera of proteases” means a fusion protein of two or more fragments derived from different parent proteases.

The term “substrate” means any molecule that can be converted catalytically by an enzyme. The term “peptide substrate” means any peptide, oligopeptide, or protein molecule of any amino acid composition, sequence or length, that contains a peptide bond that can be hydrolyzed catalytically by a protease. The peptide bond that is hydrolyzed is referred to as the “cleavage site”. Numbering of positions in the substrate is done according to the system introduced by Schlechter & Berger (Biochem. Biophys. Res. Commun. 27 (1967) 157-162). Amino acid residues adjacent N-terminal to the cleavage site are numbered P₁, P₂, P₃, etc., whereas residues adjacent C-terminal to the cleavage site are numbered P₁′, P₂′, P₃′, etc.

The term “target substrate” describes a user-defined substrate which is specifically recognized and converted by an enzyme according to the invention. The term “target peptide substrate” describes a user-defined peptide substrate. The term “target specificity” describes the qualitative and quantitative specificity of an enzyme that is capable of recognizing and converting a target substrate.

Catalytic properties of enzymes are expressed using the kinetic parameters “K_{M}” or “Michaelis Menten constant”, “k_{cat}” or “catalytic rate constant”, and “k_{cat}/K_{M}” or “catalytic efficiency”, according to the definitions of Michaelis and Menten (Fersht, A., Enzyme Structure and Mechanism, W. H. Freeman and Company, New York, 1995). The term “catalytic activity” describes quantitatively the conversion of a given substrate under defined reaction conditions.

The term “specificity” means the ability of an enzyme to recognize and convert preferentially certain substrates. Specificity can be expressed qualitatively and quantitatively. “Qualitative specificity” refers to the chemical nature of the substrate residues that are recognized by an enzyme. “Quantitative specificity” refers to the number of substrates that are accepted as substrates. Quantitative specificity can be expressed by the term s, which is defined as the negative logarithm of the number of all accepted substrates divided by the number of all possible substrates. Proteases, for example, that accept preferantially a small portion of all possible peptide substrates have a “high specificity”. Proteases that accept almost any peptide substrate have a “low specificity”. Definitions are made in accordance to WO 03/095670 which is therefore incorporated by reference. Proteases with very low specificity are also referred to as “unspecific proteases”. The term “defined specificity” refers to a certain type of specificity, i.e. to a certain target subtrate or a set of certain target substrates that are preferentially converted versus other substrates.

The term “engineered” in combination with the term “enzyme” describes an enzyme that is comprised of different components and that has features not being conferred by the individual components alone.

The term “protein scaffold” or “scaffold protein” refers to a variety of primary, secondary and tertiary polypeptide structures.

The term “peptide sequence” indicates any peptide sequence used for insertion or substitution into or combination with a protein scaffold. Peptide sequences are usually obtained by expression from DNA sequences which can be synthesized according to well-established techniques or can be obtained from natural sources. Insertion, substitution or combination of peptide sequences with the protein scaffold are generated by insertion, substitution or combination of oligonucleotides into or with a polynucleotide encoding the protein scaffold. The term “synthetic” in combination with the term “peptide sequence” refers to peptide sequences that are not present in the protein scaffold in which the peptide sequences are inserted or substituted or with which they are combined.

The term “components” in combination with the term “engineered enzyme” refers to peptide or polypeptide sequences that are combined in the engineering of such enzymes. Such components may among others comprise one or more protein scaffolds and one or more synthetic peptide sequences. The term “library of engineered enzymes” describes a mixture of engineered enzymes, whereby every single engineered enzyme is encoded by a different polynucleotide sequence. The term “gene library” indicates a library of polynucleotides that encodes the library of engineered enzymes. The term “SDR” or “Specificity determining region” refers to a synthetic peptide sequence that provides the defined specificity when combined with the protein scaffold at sites that enable the resulting enzymes to discriminate between the target substrate and one or more other substrates. Such sites are termed “SDR sites”.

The terms “tertiary structure similar to the structure of” and “similar tertiary structure” in combination with the terms “enzyme” or “protein” refer to proteins in which the type, sequence, connectivity and relative orientation of the typical secondary structural elements of a protein, e.g. alpha-helices, beta-sheets, beta-turns and loops, are similar and the proteins are therefore grouped into the same structural or topological class or fold. This includes proteins that have altered, additional or deleted structural elements of any type but otherwise unchanged topology. Examples of such structural classes are the TNF superfamily, the S1 fold or the S8 fold within the serine proteases, the GPCRs, or the α/β-barrel fold.

The term “positions that correspond structurally” indicates amino acids in proteins of similar tertiary structure that correspond structurally to each other, i.e. they are usually located within the same structural or topological element of the structure. Within the structural element they possess the same relative positions with respect to beginning and end of the structural element. If, e.g. the topological comparison of two proteins reveals two structurally corresponding sequences of different length, then amino acids within, e.g. 20% and 40% of the respective region lengths, correspond to each other structurally.

The term “library of engineered enzymes” of the present invention refers to a multiplicity of enzymes or enzyme variants, which may exist as a mixture or in isolated form.

Amino acids residues are abbreviated according to the following Table 1 either in one- or in three-letter code.

TABLE 1
Amino acid abbreviations

| | |
|---|---|
| Abbreviations | Amino acid |

| | | |
|---|---|---|
| A | Ala | Alanine |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophane |
| Y | Tyr | Tyrosine |

### DETAILED DESCRIPTION OF THE INVENTION

The present invention provides engineered proteins with novel functions. In particular, the invention provides enzymes with user-definable specificities. In a particular embodiment, the invention provides proteases with user-definable specificities. Besides, the invention provides applications of such enzymes with novel, user-definable specificities for therapeutic, research, diagnostic, nutritional, personal care or industrial purposes. Moreover, the invention provides a method for generating enzymes with specificities that are not present in the components used for the engineering of such enzymes. In particular, the invention is directed to the generation of enzymes that have sequences that are essentially identical to mammalian, especially human enzymes but have different specificities . Moreover, the invention provides libraries of specific engineered enzymes with corresponding specificities encoded genetically, a method for the generation of libraries of specific engineered enzymes with corresponding specificities encoded genetically, and the application of such libraries for technical, diagnostic, nutritional, personal care or research purposes.

A first aspect of the invention is directed to the application of engineered enzymes with specificities for therapeutic, research, diagnostic, nutritional, personal care or industrial purposes. The application comprises at least the following steps:

(a) identification of a target peptide substrate whose hydrolysis has a positive effect in connection with the intended purpose, such as curing a disease, diagnosing a disease, processing of ingredients for human or animal nutrition, or other technical processes;

(b) provision of an engineered enzyme, the enzyme being specific for the target peptide identified in step (a); and

(c) use of the enzyme as provided in step (b) for the intended purpose.

In a first variant of this aspect of the invention, the engineered enzyme is used as a therapeutic means to inactivate a disease-related target substrate. This application comprises at least the following steps:

(a) identification of a target substrate whose function is connected to a disease and whose inactivation has a positive effect in connection with the disease, and determination of a target site within the target substrate characterized by the fact that modification at the target site leads to the inactivation of the target substrate;

(b) provision of an engineered enzyme, the enzyme being specific for the target site identified in step (a); and

(c) use of the enzyme for the inactivation of the target substrate inside or outside the human body.

Preferably, the scaffold is a protease and the modification is hydrolysis of a target site in a protein target. Preferably, the hydrolysis leads to the activation or inactivation of the peptide or protein target. Potential peptide or protein targets include soluble proteins, in particular cytokines, such as proteins of the TNF-superfamily, interleukines, interferons, chemokines and growth factors; hormones; toxins; enzymes, such as oxidoreductases, transferases, hydrolases, lyases, isomerases and ligases; structural proteins, such as collagen; and immunoglobulins; or membrane associated proteins, in particular single pass transmembrane proteins; multipass transmembrane proteins, such as G-protein coupled receptors, ion channels and transporters; lipid-anchored membrane proteins and GPI-anchored membrane proteins.

In a first embodiment of this variant the engineered enzyme is a protease and is capable of hydrolysing human tumor necrosis factor-alpha (hTNF-α). The enzymes or the fusion protein can thus be used for preparing medicaments for the treatment of diseases, such as, but not limited to, rheumatoid arthritis, inflammatory bowel diseases, psoriasis, Crohn's disease, Ulcerative colitis, diabetes type II, classical Hodgkin's Lymphoma (cHL), Grave's disease, Hashimoto's thyroiditis, Sjogren's Syndrome, systemic lupus erythematosus, multiple sclerosis, Systemic inflammatory response syndrome (SIRS) which leads to distant organ damage and multiple organ dysfunction syndrome (MODS), eosinophilia, neurodegenerative disease, stroke, closed head injury, encephalitis, CNS disorders, asthma, rheumatoid arthritis, sepsis, vasodilation, intravascular coagulation and multiple organ failure, as well as other diseases connected with hTNF-α. Preferably, said enzyme or said fusion protein is capable of specifically inactivating hTNF-α (SEQ ID NO:96). More preferably said enzyme or said fusion protein is capable of hydrolysing the peptide bonds between positions 31/32, 32/33, 44/45, 45/46, 87/88, 128/129, 130/131, 140/141 and/or 141/142 (most preferred between positions 31/32, 32/33 and/or 45/46) in hTNF-□, or a peptide bond in proximity to these positions in hTNF-□, or peptide bonds in protein targets related to hTNF-□ between positions having structural homology or sequence homology to these positions. In this embodiment it is most preferred that the protease has the a sequence shown in SEQ ID NO:74, SEQ ID NO:75 and is capable of hydrolysing hTNF-α at positions 31/32 and/or 32/33.

In a second embodiment of this variant the enzyme is a protease and is capable of hydrolysing human Tumor necrosis factor ligand superfamily member 5 (hCD40-L). The enzymes or the fusion protein can thus be used for preparing medicaments for the treatment of diseases, such as, but not limited to, systemic lupus erythematosus and classical Hodgkin's Lymphoma (cHL), as well as other diseases connected with hCD40-L. Preferably, said enzyme or said fusion protein is capable of specifically inactivating hCD40-L (SEQ ID NO:143). More preferably said enzyme or said fusion protein is capable of hydrolysing the peptide bonds between positions 133/134, 145/146, 165/166, 200/201, 201/202, 207/208 and/or 216/217 (most preferred between positions 133/134, 165/166, 201/202 and/or 216/217) in hCD40-L, or a peptide bond in proximity to these positions in hCD40-L, or peptide bonds in protein targets related to hCD40-L at positions having structural homology or sequence homology to these positions.

In a third embodiment of this variant the enzyme is a protease and is capable of hydrolysing human Macrophage migration inhibitory factor (hMIF). The enzymes or the fusion protein can thus be used for preparing medicaments for the treatment of diseases, such as, but not limited to, inflammatory diseases, as well as other diseases connected with hMIF. Preferably, said enzyme or said fusion protein is capable of specifically inactivating hMIF (SEQ ID NO:109). More preferably said engineered or said fusion protein is capable of hydrolysing the peptide bonds between positions 16/17, 44/45, 66/67, 73/74, 77/78, 88/89, 92/93 and/or 100/101 (most preferred between positions 16/17 and/or 92/93) in hMIF, or a peptide bond in proximity to these positions in hMIF, or peptide bonds in protein targets related to hMIF at positions having structural homology or sequence homology to these positions.

In a fourth embodiment of this variant the enzyme is a protease and is capable of hydrolysing human Interleukin-1 beta precursor (hIL-1 beta). The enzymes or the fusion protein can thus be used for preparing medicaments for the treatment of diseases, such as, but not limited to, diabetes, brain inflammation in cancer, arthritis, autoimmune and inflammatory diseases, as well as other diseases connected with hIL-1 beta. Preferably, said enzyme or said fusion protein is capable of specifically inactivating hIL-1 beta (SEQ ID NO:112). More preferably said enzyme or said fusion protein is capable of hydrolysing the peptide bonds between positions 24/25, 35/36, 46/47, 54/55, 74/75, 75/76, 76/77, 77/78, 86/87, 88/89, 93/94, 94/95, 97/98 and/or 150/151 (most preferred between positions 35/36, 75/76, 76/77, 88/89, 93/94, 94/95 and/or 150/151) in hIL-1 beta, or a peptide bond in proximity to these positions in hIL-1 beta, or peptide bonds in protein targets related to hIL- 1 beta at positions having structural homology or sequence homology to these positions.

In a fifth embodiment of this variant the enzyme is a protease and is capable of hydrolysing human Interleukin 2 (hIL-2). The enzymes or the fusion protein can thus be used for preparing medicaments for the treatment of diseases, such as, but not limited to, T-cell leukemia and hairy cell leukemia, Crohn's disease, Ulcerative colitis, Grave's disease, Hashimoto's thyroiditis, Sjogren's syndrome, systemic lupus erythematosus, multiple sclerosis, asthma and chronic obstructive pulmonary and classical Hodgkin's Lymphoma (cHL), as well as other diseases connected with hIL-2. Preferably, said enzyme or said fusion protein is capable of specifically inactivating hIL-2 (SEQ ID NO:99). More preferably said enzyme or said fusion protein is capable of hydrolysing the peptide bonds between positions 20/21, 32/33, 38/39, 43/44, 45/46 48/49, 49/50, 54/55, 64/65, 76/77, 83/84, 84/85, 107/108, 109/110 and/or 120/121 (most preferred between positions 109/110) in hIL-2, or a peptide bond in proximity to these positions in hIL-2, or peptide bonds in protein targets related to hIL-2 at positions having structural homology or sequence homology to these positions.

In a sixth embodiment of this variant the enzyme is a protease and is capable of hydrolysing human Interleukin 3 (hIL-3). The enzymes or the fusion protein can thus be used for preparing medicaments for the treatment of diseases, such as, but not limited to, classical Hodgkin's Lymphoma (cHL) and eosinophilia, as well as other diseases connected withh IL-3. Preferably, said enzyme or said fusion protein is capable of specifically inactivating hIL-3 (SEQ ID NO: 148). More preferably said enzyme or said fusion protein is capable of hydrolysing the peptide bonds between positions 21/22, 28/29, 36/37, 44/45, 46/47, 51/52, 63/64, 66/67, 79/80, 94/95, 101/102, 108/109 and/or 109/110 (most preferred between positions 21/22, 28/29, 46/47, 63/64, 66/67, 79/80 and/or 101/102) in hIL-3, or a peptide bond in proximity to these positions in hIL-3, or peptide bonds in protein targets related to hIL-3 at positions having structural homology or sequence homology to these positions.

In a seventh embodiment of this variant the enzyme is a protease and is capable of hydrolysing human Interleukin 4 (hIL-4). The enzymes or the fusion protein can thus be used for preparing medicaments for the treatment of diseases, such as, but not limited to, classical Hodgkin's Lymphoma (cHL), Grave's disease, Hashimoto's thyroiditis, Sjogren's syndrome, Asthma, chronic obstructive pulmonary disease and allergic inflammatory reactions, as well as other diseases connected with hIL-4. Preferably, said enzyme or said fusion protein is capable of specifically inactivating hIL-4 (SEQ ID NO: 118). More preferably said enzyme or said fusion protein is capable of hydrolysing the peptide bonds between positions 4/5, 12/13, 31/32, 37/38, 61/62, 62/63, 64/65, 91/92, 102/103, 121/122 and/or 126/127 (most preferred between positions 4/5, 61/62, 62/63, 64/65 and/or 121/122) in hIL-4, or a peptide bond in proximity to these positions in hIL-4, or peptide bonds in protein targets related to hIL-4 at positions having structural homology or sequence homology to these positions.

In an eighth embodiment of this variant the enzyme is a protease and is capable of hydrolysing human Interleukin-5 (hIL-5). The enzymes or the fusion protein can thus be used for preparing medicaments for the treatment of diseases, such as, but not limited to, classical Hodgkin's Lymphoma (cHL), asthma, chronic obstructive pulmonary disease, eosinophilia, allergic inflammatory diseases, as well as other diseases connected with hIL-5. Preferably, said enzyme or said fusion protein is capable of specifically inactivating hIL-5 (SEQ ID NO: 133). More preferably said enzyme or said fusion protein is capable of hydrolysing the peptide bonds between positions 12/13, 32/33, 67/68, 76/77, 77/78, 80/81, 83/84, 84/85, 85/86, 90/91, 91/92, 92/93 and/or 98/99 (most preferred between positions 90/91, 91/92, 92/93 and/or 98/99) in hIL-5, or a peptide bond in proximity to these positions in hIL-5, or peptide bonds in protein targets related to hIL-5 at positions having structural homology or sequence homology to these positions.

In a ninth embodiment of this variant the enzyme is a protease and is capable of hydrolysing human Interleukin-6 (hIL-6). The enzymes or the fusion protein can thus be used for preparing medicaments for the treatment of diseases, such as, but not limited to, classical Hodgkin's Lymphoma (cHL), breast cancer, renal cell carcinoma, multiple myeloma, lymphoma, leukemia, Grave's disease, Hashimoto's thyroiditis, Sjogren's syndrome, systemic lupus erythematosus, Systemic inflammatory response syndrome (SIRS) which leads to distant organ damage and multpile organ dysfunction syndrome (MODS), chronic obstructive pulmonary disease (COPD), Castleman's diseases, inflammatory bowel diseases, Crohn's disease, as well as other diseases connected with hIL-6. Preferably, said enzyme or said fusion protein is capable of specifically inactivating hIL-6 (SEQ ID NO:134). More preferably said enzyme or said fusion protein is capable of hydrolysing the peptide bonds between positions 32/33, 35/36, 55/56, 71/72, 129/130, 130/131, 132/133, 135/136, 141/142, 161/162, 180/181 and/or 183/184 (most preferred between positions 135/136 and/or 141/142) in hIL-6, or a peptide bond in proximity to these positions in hIL-6, or peptide bonds in protein targets related to hIL-6 at positions having structural homology or sequence homology to these positions.

In a tenth embodiment of this variant the enzyme is a protease and is capable of hydrolysing human Interleukin 8 (hIL-8). The enzymes or the fusion protein can thus be used for preparing medicaments for the treatment of diseases, such as, but not limited to, Crohn's disease, Ulcerative colitis, classical Hodgkin's Lymphoma (cHL), Systemic inflammatory response syndrome (SIRS) which leads to distant organ damage and multple organ dysfunction syndrome (MODS), chronic obstructive pulmonary disease (COPD), endometriosis, psoriasis and atherosclerotic lesions, as well as other diseases connected with hIL-8. Preferably, said enzyme or said fusion protein is capable of specifically inactivating hIL-8 (SEQ ID NO: 100). More preferably said enzyme or said fusion protein is capable of hydrolysing the peptide bonds between positions 1/12, 15/16, 45/46, 47/48, 52/53, 54/55, 60/61, 64/65 and/or 67/68 (most preferred between positions 45/46) in hIL-8, or a peptide bond in proximity to these positions in hIL-8, or peptide bonds in protein targets related to hIL-8 at positions having structural homology or sequence homology to these positions.

In an eleventh embodiment of this variant the enzyme is a protease and is capable of hydrolysing human Interleukin-10 (hIL-10). The enzymes or the fusion protein can thus be used for preparing medicaments for the treatment of diseases, such as, but not limited to, classical Hodgkin's Lymphoma (cHL) and diseases related to the suppression of cytotoxic T-cells, as well as other diseases connected with hIL-10. Preferably, said enzyme or said fusion protein is capable of specifically inactivating hIL-10 (SEQ ID NO:135). More preferably said enzyme or said fusion protein is capable of hydrolysing the peptide bonds between positions 24/25, 25/26, 27/28, 28/29, 40/41, 44/45, 49/50, 57/58, 59/60, 84/85, 86/87, 106/107, 107/108, 110/111, 130/131, 134/135, 137/138, 138/139 and/or 144/145 (most preferred between positions 24/25, 27/28, 44/45, 49/50, 86/87, 137/138 and/or 144/145) in hIL-10, or a peptide bond in proximity to these positions in hIL-10, or peptide bonds in protein targets related to hIL-10 at positions having structural homology or sequence homology to these positions.

In a twelfth embodiment of this variant the enzyme is a protease and is capable of hydrolysing human Interleukin 12 beta chain (hIL-12β). The enzymes or the fusion protein can thus be used for preparing medicaments for the treatment of diseases, such as, but not limited to, Crohn's disease and classical Hodgkin's Lymphoma (cHL), as well as other diseases connected with hIL-12β. Preferably, said enzyme or said fusion protein is capable of specifically inactivating hIL-12β (SEQ ID NO:97). More preferably said enzyme or said fusion protein is capable of hydrolysing the peptide bonds between positions 14/15, 18/19, 29/30, 34/35, 87/88, 99/100, 102/103, 104/105, 161/162, 174/175, 222/223, 225/226, 228/229, 238/239, 268/269 and/or 293/294 (most preferred between positions 18/19, 34/35, 87/88 and/or 161/162) in hIL-12β, or a peptide bond in proximity to these positions in hIL- 12B, or peptide bonds in protein targets related to hIL-12β at positions having structural homology or sequence homology to these positions.

In a thirteenth embodiment of this variant the enzyme is a protease and is capable of hydrolysing human Interleukin 13 (hIL-13). The enzymes or the fusion protein can thus be used for preparing medicaments for the treatment of cancer, such as, but not limited to, classical Hodgkin's Lymphoma (cHL), eosinophilia, asthma, chronic obstructive pulmonary disease, fibrosis, psoriasis, atopic dermatitis and Ulcerative colitis, as well as other diseases connected with hIL-13. Preferably, said enzyme or said fusion protein is capable of specifically inactivating hIL-13 (SEQ ID NO:119). More preferably said enzyme or said fusion protein is capable of hydrolysing the peptide bonds between positions 25/26, 62/63, 65/66, 86/87, 87/88, 98/99, 108/109 and/or 111/112 (most preferred between positions 87/88) in hIL-13, or a peptide bond in proximity to these positions in hIL-13, or peptide bonds in protein targets related to hIL-13 at positions having structural homology or sequence homology to these positions.

In a fourteenth embodiment of this variant the enzyme is a protease and is capable of hydrolysing human Interleukin 18 (hIL-18). The enzymes or the fusion protein can thus be used for preparing medicaments for the treatment of diseases, such as, but not limited to, Crohn's disease, inflammation liver injuries, pulmonary tuberculosis, plural tuberculosis and rheumatoid arthritis, as well as other diseases connected with hIL-18. Preferably, said enzyme or said fusion protein is capable of specifically inactivating hIL-18 (SEQ ID NO:98). More preferably said enzyme or said fusion protein is capable of hydrolysing the peptide bonds between positions 17/18, 32/33, 37/38, 39/40, 40/41, 53/54, 58/59, 79/80, 90/91, 93/94, 98/99, 110/111, 120/121, 123/124, 131/132, 132/133, 142/143, 147/148 and/or 157/158 (most preferred between positions 37/38, 132/133, 142/143 and/or 157/158) in hIL-18, or a peptide bond in proximity to these positions in hIL-18, or peptide bonds in protein targets related to hIL-18 at positions having structural homology or sequence homology to these positions.

In a fifteenth embodiment of this variant the enzyme is a protease and is capable of hydrolysing human Interferon-gamma (hIFN-gamma). The enzymes or the fusion protein can thus be used for preparing medicaments for the treatment of diseases, such as, but not limited to, classical Hodgkin's Lymphoma (cHL), Crohn's disease and type I diabetes, as well as other diseases connected with hIFN-gamma. Preferably, said enzyme or said fusion protein is capable of specifically inactivating hIFN-gamma (SEQ ID NO:137). More preferably said enzyme or said fusion protein is capable of hydrolysing the peptide bonds between positions 2/3, 6/7, 13/14, 21/22, 24/25, 34/35, 36/37, 37/38, 62/63, 68/69, 83/84, 86/87, 90/91, 102/103, 107/108 and/or 108/109 (most preferred between positions 13/14, 24/25, 37/38, 62/63, 68/69, 102/103 and/or 107/108) in hIFN-gamma, or a peptide bond in proximity to these positions in hIFN-gamma, or peptide bonds in protein targets related to hIFN-gamma at positions having structural homology or sequence homology to these positions.

In a sixteenth embodiment of this variant the enzyme is a protease and is capable of hydrolysing human small inducible cytokine A2 (hCCL2). The enzymes or the fusion protein can thus be used for preparing medicaments for the treatment of diseases, such as, but not limited to, Crohn's disease and Ulcerative colitis, as well as other diseases connected with hCCL2. Preferably, said enzyme or said fusion protein is capable of specifically inactivating hCCL2 (SEQ ID NO: 102). More preferably said enzyme or said fusion protein is capable of hydrolysing the peptide bonds between positions 3/4, 13/14, 18/19, 19/20, 24/25, 29/30, 38/39, 54/55, 56/57, 58/59, 62/63, 65/66 and/or 68/69 (most preferred between positions 19/20, 29/30, 38/39, 54/55 and/or 62/63) in hCCL2, or a peptide bond in proximity to these positions in hCCL2, or peptide bonds in protein targets related to hCCL2 at positions having structural homology or sequence homology to these positions.

In a seventeenth embodiment of this variant the enzyme is a protease and is capable of hydrolysing human Eotaxin (hCCL11). The enzymes or the fusion protein can thus be used for preparing medicaments for the treatment of diseases, such as, but not limited to, Crohn's disease and Ulcerative colitis, classical Hodgkin's Lymphoma (cHL), chronic pathophysiologic dysfunction, characterized by an influx mainly of Th2 cells, and eosinophilia, as well as other diseases connected with hCCL11. Preferably, said enzyme or said fusion protein is capable of specifically inactivating hCCL11 (SEQ ID NO:101). More preferably said enzyme or said fusion protein is capable of hydrolysing the peptide bonds between positions 11/12, 16/17, 17/18, 22/23, 27/28, 33/34, 44/45, 47/48, 48/49, 52/53, 54/55, 56/57, 60/61, 66/67 and/or 73/74 (most preferred between positions 48/49 and/or 66/67) in hCCL11, or a peptide bond in proximity to these positions in hCCL11, or peptide bonds in protein targets related to hCCL11 at positions having structural homology or sequence homology to these positions.

In an eighteenth embodiment of this variant the enzyme is a protease and is capable of hydrolysing human Vascular endothelial growth factor (hVEGF). The enzymes or the fusion protein can thus be used for preparing medicaments for the treatment of diseases, .such as, but not limited to, all solid tumors and metastatic solid tumors, inflammatory breast cancer, as well as other diseases connected with hVEGF. Preferably, said enzyme or said fusion protein is capable of specifically inactivating hVEGF (SEQ ID NO: 103). More preferably said enzyme or said fusion protein is capable of hydrolysing the peptide bonds between positions 16/17, 19/20, 23/24, 34/35, 41/42, 56/57, 62/63, 63/64, 64/65, 65/66, 82/83, and/or 84/85 (most preferred between positions 23/24, 41/42, 63/64, 82/83 and/or 84/85) in hVEGF, or a peptide bond in proximity to these positions in hVEGF, or peptide bonds in protein targets related to hVEGF at positions having structural homology or sequence homology to these positions.

In a ninteenth embodiment of this variant the enzyme is a protease and is capable of hydrolysing human Transforming growth factor beta 1 (hTGF-β1). The enzymes or the fusion protein can thus be used for preparing medicaments for the treatment of diseases, such as, but not limited to, a variety of cancers, including breast cancer, colorectal cancer and classical Hodgkin's Lymphoma (cHL), fibrosis, suppression of cell-mediated immunity, glaucoma, diffuse systemic sclerosis as well as other diseases connected with hTGF-β1. Preferably, said enzyme or said fusion protein is capable of specifically inactivating hTGF-β1 (SEQ ID NO: 104). More preferably said enzyme or said fusion protein is capable of hydrolysing the peptide bonds between positions 23/24, 25/26, 26/27, 27/28, 37/38, 55/56 and/or 94/95 (most preferred between positions 25/26, 55/56 and/or 94/95) in hTGF-β1, or a peptide bond in proximity to these positions in hTGF-β1, or peptide bonds in protein targets related to hTGF-β1 at positions having structural homology or sequence homology to these positions.

In a twentieth embodiment of this variant the enzyme is a protease and is capable of hydrolysing human Somatotropin (human Growth hormone; hGH). The enzymes or the fusion protein can thus be used for preparing medicaments for the treatment of diseases, such as, but not limited to, acromegaly, diabetes and diabetic kidney disease including renal hypertrophy and glomerular enlargement and cardiovascular disorders, as well as other diseases connected with hGH. Preferably, said enzyme or said fusion protein is capable of specifically inactivating hGH (SEQ ID NO: 121). More preferably said enzyme or said fusion protein is capable of hydrolysing the peptide bonds between positions 8/9, 16/17, 19/20, 26/27, 33/34, 38/39, 41/42, 70/71, 77/78, 94/95, 103/104, 112/113, 115/116, 116/117, 130/131, 147/148, 154/155 and/or 178/179 (most preferred between positions 112/113, 147/148 and/or 154/155) in hGH, or a peptide bond in proximity to these positions in hGH, or peptide bonds in protein targets related to hGH at positions having structural homology or sequence homology to these positions.

In a twenty-first embodiment of this variant the enzyme is a protease and is capable of hydrolysing human Insulin-like growth factor II (hIGF-II). The enzymes or the fusion protein can thus be used for preparing medicaments for the treatment of diseases, such as, but not limited to, diabetes and diabetic kidney disease, as well as other diseases connected with hIGF-II. Preferably, said enzyme or said fusion protein is capable of specifically inactivating hIGF-II (SEQ ID NO: 122). More preferably said enzyme or said fusion protein is capable of hydrolysing the peptide bonds between positions 15/16, 23/24, 24/25, 34/35, 37/38, 38/39, 48/49 and/or 49/50 (most preferred between positions 23/24) in hIGF-II, or a peptide bond in proximity to these positions in hIGF-II, or peptide bonds in protein targets related to hIGF-II at positions having structural homology or sequence homology to these positions.

In a twenty-second embodiment of this variant the enzyme is a protease and is capable of hydrolysing human Hepatocyte growth factor (hHGF). The enzymes or the fusion protein can thus be used for preparing medicaments for the treatment of diseases, such as, but not limited to, angiogenic disorders and hepatocellular carcinoma, as well as other diseases connected with hHGF. Preferably, said enzyme or said fusion protein is capable of specifically inactivating hHGF (SEQ ID NO:120). More preferably said enzyme or said fusion protein is capable of hydrolysing the peptide bonds between positions 54/55, 60/61, 62/63, 63/64, 68/69, 76/77, 112/113, 123/124, 134/135, 168/169, 198/199 and/or 202/203 (most preferred between positions 63/64, 68/69, 76/77, 168/169 and/or 202/203) in hHGF, or a peptide bond in proximity to these positions in hHGF, or peptide bonds in protein targets related to hHGF at positions having structural homology or sequence homology to these positions.

In a twenty-third embodiment of this variant the enzyme is a protease and is capable of hydrolysing human hInsulin (hInsulin). The enzymes or the fusion protein can thus be used for preparing medicaments for the treatment of diseases, such as, but not limited to, insulin overdosage, as well as other diseases connected with hInsulin. Preferably, said enzyme or said fusion protein is capable of specifically inactivating hInsulin B chain (SEQ ID NO: 105) and/or hInsulin A chain (SEQ ID NO: 106). More preferably said enzyme or said fusion protein is capable of hydrolysing the peptide bonds between positions 16/17 and/or 22/23 in hInsulin B and/or between position 14/15 in Insulin A, or a peptide bond in proximity to these positions in hInsulin A or B, or peptide bonds in protein targets related to hInsulin A or B at positions having structural homology or sequence homology to these positions.

In a twenty-fourth embodiment of this variant the enzyme is a protease and is capable of hydrolysing human hGhrelin (hGhrelin). The enzymes or the fusion protein can thus be used for preparing medicaments for the treatment of diseases, such as, but not limited to, obesity, as well as other diseases connected with hGhrelin. Preferably, said enzyme or said fusion protein is capable of specifically inactivating hGhrelin (SEQ ID NO: 107). More preferably said enzyme or said fusion protein is capable of hydrolysing the peptide bonds between positions 1/2, 2/3, 3/4 and/or 4/5 in hGhrelin, or a peptide bond in proximity to these positions in hGhrelin, or peptide bonds in protein targets related to hGhrelin at positions having structural homology or sequence homology to these positions.

In a twenty-fifth embodiment of this variant the enzyme is a protease and is capable of hydrolysing human angiotensinogen (angiotensin). The enzymes or the fusion protein can thus be used for preparing medicaments for the treatment of diseases, such as, but not limited to, essential hypertension, as well as other diseases connected with angiotensin. Preferably, said enzyme or said fusion protein is capable of specifically inactivating angiotensin (SEQ ID NO:108). More preferably said enzyme or said fusion protein is capable of hydrolysing the peptide bonds between positions 1/2, 3/4 and/or 7/8 (most preferred between positions 3/4) in angiotensin, or a peptide bond in proximity to these positions in angiotensin, or peptide bonds in protein targets related to angiotensin at positions having structural homology or sequence homology to these positions.

In a twenty-sixth embodiment of this variant the enzyme is a protease and is capable of hydrolysing human leptin precursor (leptin). The enzymes or the fusion protein can thus be used for preparing medicaments for the treatment of diseases, such as, but not limited to, obesity, as well as other diseases connected with leptin. Preferably, said enzyme or said fusion protein is capable of specifically inactivating leptin (SEQ ID NO:127). More preferably said enzyme or said fusion protein is capable of hydrolysing the peptide bonds between positions 8/9, 9/10, 15/16, 23/24, 40/41, 53/54, 71/72, 85/86, 94/95, 108/109 and/or 141/142 (most preferred between positions 9/10, 40/41, 71/72, 94/95 and/or 108/109) in leptin, or a peptide bond in proximity to these positions in leptin, or peptide bonds in protein targets related to leptin at positions having structural homology or sequence homology to these positions.

In a twenty-seventh embodiment of this variant the enzyme is a protease and is capable of hydrolysing Protective antigen (PA-83). The enzymes or the fusion protein can thus be used for preparing medicaments for the treatment of diseases, such as, but not limited to, anthrax infection, as well as other diseases connected with PA-83. Preferably, said enzyme or said fusion protein is capable of specifically inactivating PA-83 (SEQ ID NO:123). More preferably said enzyme or said fusion protein is capable of hydrolysing the peptide bonds between positions 72/73, 73/74, 92/93, 93/94, 131/132, 149/150, 178/179, 213/214, 214/215, 387/388, 425/426, 426/427, 427/428, 453/454, 520/521, 608/609, 617/618, 671/672, 679/680, 680/681, 683/684 and/or 684/685 (most preferred between positions 72/73, 73/74, 93/94, 149/150, 387/388, 425/426, 427/428 and/or 683/684) in PA-83, or a peptide bond in proximity to these positions in PA-83, or peptide bonds in protein targets related to PA-83 at positions having structural homology or sequence homology to these positions.

In a twenty-eighth embodiment of this variant the enzyme is a protease and is capable of hydrolysing human plasminogen (plasminogen). The enzymes or the fusion protein can thus be used for preparing medicaments for the treatment of diseases, such as, but not limited to, thrombosis, as well as other diseases connected with plasminogen. Preferably, said enzyme or said fusion protein is capable of specifically inactivating plasminogen (SEQ ID NO:140). More preferably said enzyme or said fusion protein is capable of hydrolysing the peptide bond between position 580/581 in plasminogen, or a peptide bond in proximity to this position in plasminogen, or peptide bonds in protein targets related to plasminogen at positions having structural homology or sequence homology to these positions.

In a twenty-ninth embodiment of this variant the enzyme is a protease and is capable of hydrolysing human Prothrombin (thrombin). The enzymes or the fusion protein can thus be used for preparing medicaments for the treatment of diseases, such as, but not limited to, bleeding, as well as other diseases connected with thrombin. Preferably, said enzyme or said fusion protein is capable of specifically inactivating thrombin (SEQ ID NO:149). More preferably said enzyme or said fusion protein is capable of hydrolysing the peptide bonds between positions 198/199, 327/328, 363/364 (most preferred between positions 327/328 and/or 363/364) in thrombin, or a peptide bond in proximity to these positions in thrombin, or peptide bonds in protein targets related to thrombin at positions having structural homology or sequence homology to these positions

In a thirty embodiment of this variant the enzyme is a protease and is capable of hydrolysing human beta-secretase. The enzymes or the fusion protein can thus be used for preparing medicaments for the treatment of diseases, such as, but not limited to, Alzheimer, as well as other diseases connected with human beta-secretase precursor. Preferably, said enzyme or said fusion protein is capable of specifically inactivating human beta-secretase precursor (SEQ ID NO:139). More preferably said enzyme or said fusion protein is capable of hydrolysing the peptide bonds between positions 61/62, 64/65, 159/160, 238/239, 239/240, 246/247, 256/257, 330/331 and/or 365/366 (most preferred between positions 61/62, 246/247 and/or 365/366) in human beta-secretase precursor, or a peptide bond in proximity to these positions in human beta-secretase precursor, or peptide bonds in protein targets related to human beta-secretase precursor at positions having structural homology or sequence homology to these positions.

In a thirty-first embodiment of this variant the enzyme is a protease and is capable of hydrolysing human matrix metalloproteinase-2 (hMMP-2). The enzymes or the fusion protein can thus be used for preparing medicaments for the treatment of diseases, such as, but not limited to, a variety of cancers including bladder cancer, breast tumor cancer, gastric cancer and lung cancer, as well as other diseases connected with hMMP-2. Preferably, said enzyme or said fusion protein is capable of specifically inactivating hMMP-2 (SEQ ID NO: 131). More preferably said enzyme or said fusion protein is capable of hydrolysing the peptide bonds between positions 62/63, 68/69, 75/76, 76/77, 79/80, 88/89, 110/111, 112/113, 115/116, 120/121, 164/165, 254/255, 267/268, 296/297, 324/325, 325/326, 382/383, 383/384, 470/471, 500/501, 550/551, 564/565, 595/596, 597/598, 608/609, 646/647, 649/650 and/or 650/651 (most preferred between positions 68/69, 115/116, 120/121, 164/165, 325/326, 383/384, 470/471, 500/501, 595/596, 608/609 and/or 650/651) in hMMP-2, or a peptide bond in proximity to these positions in hMMP-2, or peptide bonds in protein targets related to hMMP-2 at positions having structural homology or sequence homology to these positions.

In a thirty-second embodiment of this variant the enzyme is a protease and is capable of hydrolysing human matrix metalloproteinase-9 (hMMP-9). The enzymes or the fusion protein can thus be used for preparing medicaments for the treatment of diseases, such as, but not limited to, a variety of cancers including bladder cancer, breast tumor cancer, gastric cancer and lung cancer, as well as other diseases connected with hMMP-9. Preferably, said enzyme or said fusion protein is capable of specifically inactivating hMMP-9 (SEQ ID NO:132). More preferably said enzyme or said fusion protein is capable of hydrolysing the peptide bonds between positions 41/42, 42/43, 106/107, 113/114, 134/135, 160/161, 162/163, 163/164, 222/223, 226/227, 265/266, 266/267, 267/268, 284/285, 309/310, 321/322, 322/323, 324/325, 356/357, 380/381, 433/434 and/or 440/441 (most preferred between positions 160/161, 163/164, 226/227, 284/285, 321/322, 322/323 and/or 433/434) in hMMP-9, or a peptide bond in proximity to these positions in hMMP-9, or peptide bonds in protein targets related to hMMP-9 at positions having structural homology or sequence homology to these positions.

In a thirty-third embodiment of this variant the enzyme is a protease and is capable of hydrolysing HIV membrane glycoprotein (GP120). The enzymes or the fusion protein can thus be used for preparing medicaments for the treatment of diseases, such as, but not limited to, AIDS or HIV infection, as well as other diseases connected with GP120 or HIV infection. Preferably, said enzyme or said fusion protein is capable of specifically inactivating GP120 (SEQ ID NO:124). More preferably said enzyme or said fusion protein is capable of hydrolysing the peptide bonds between positions 97/98, 99/100, 107/108, 113/114, 117/118, 227/228, 231/233, 279/280, 335/336, 337/338, 368/369, 412/413, 419/420, 429/430, 444/445, 457/458, 474/475, 476/477, 477/478, 485/486 and/or 490/491 (most preferred between positions 99/100, 368/369, 412/413, 419/420, 444/445 and/or 490/491) in GP120, or a peptide bond in proximity to these positions in GP120, or peptide bonds in protein targets related to GP120 at positions having structural homology or sequence homology to these positions.

In a thirty-fourth embodiment of this variant the enzyme is a protease and is capable of hydrolysing human Cytotoxic T-lymphocyte protein 4 (hCTLA-4). The enzymes or the fusion protein can thus be used for preparing medicaments for the treatment of diseases, such as, but not limited to, breast cancer, as well as other diseases connected with hCTLA-4. Preferably, said enzyme or said fusion protein is capable of specifically inactivating hCTLA-4 (SEQ ID NO:144). More preferably said enzyme or said fusion protein is capable of hydrolysing the peptide bonds between positions 14/15, 28/29, 33/34, 38/39, 41/42, 62/63, 72/73, 85/86, 95/96, 100/101, 105/106, 119/120, 125/126 and/or 127/128 (most preferred between positions 14/15, 28/29, 38/39, 41/42, 62/63 and/or 85/86) in hCTLA-4, or a peptide bond in proximity to these positions in hCTLA-4, or peptide bonds in protein targets related to hCTLA-4 at positions having structural homology or sequence homology to these positions.

In a thirty-fifth embodiment of this variant the enzyme is a protease and is capable of hydrolysing human Integrin alpha-2 (hVLA-2). The enzymes or the fusion protein can thus be used for preparing medicaments for the treatment of diseases, such as, but not limited to, renal tumors, uveal melanomas and gastrointestinal tumors, as well as other diseases connected with hVLA-2. Preferably, said enzyme or said fusion protein is capable of specifically inactivating hVLA-2 (SEQ ID NO: 147). More preferably said enzyme or said fusion protein is capable of hydrolysing the peptide bonds between positions 160/161, 174/175, 201/202, 219/220, 231/232, 232/233, 233/234, 243/244, 259/260, 264/265, 268/269, 288/289, 292/293, 294/295, 298/299, 301/302, 310/311 and/or 317/318 (most preferred between positions 160/161, 174/175, 201/202, 219/220, 243/244, 264/265, 292/293 and/or 294/295) in hVLA-2, or a peptide bond in proximity to these positions in hVLA-2, or peptide bonds in protein targets related to hVLA-2 at positions having structural homology or sequence homology to these positions.

In a thirty-sixth embodiment of this variant the enzyme is a protease and is capable of hydrolysing human Vascular endothelial growth factor receptor 1 (hVEGFR 1). The enzymes or the fusion protein can thus be used for preparing medicaments for the treatment of diseases, such as, but not limited to, solid tumors and metastatic solid tumors, astrocytic brain tumors, pancreatic cancer, metastatic renal cancer, metastatic solid tumors, as well as other diseases connected with hVEGFR 1. Preferably, said enzyme or said fusion protein is capable of specifically inactivating hVEGFR 1 (SEQ ID NO: 114). More preferably said enzyme or said fusion protein is capable of hydrolysing the peptide bonds between positions 189/190, 190/191, 224/225 and/or 331/332 (most preferred between positions 189/190 and/or 331/332) in hVEGFR 1, or a peptide bond in proximity to these positions in hVEGFR 1, or peptide bonds in protein targets related to hVEGFR 1 at positions having structural homology or sequence homology to these positions.

In a thirty-seventh embodiment of this variant the enzyme is a protease and is capable of hydrolysing human Vascular endothelial growth factor receptor 2 (hVEGFR 2). The enzymes or the fusion protein can thus be used for preparing medicaments for the treatment of diseases, such as, but not limited to, solid tumors and metatstatic solid tumors, pancreatic cancer, metastatic renal cancer, metastatic CRC, as well as other diseases connected with hVEGFR 2. Preferably, said enzyme or said fusion protein is capable of specifically inactivating hVEGFR 2 (SEQ ID NO: 115). More preferably said enzyme or said fusion protein is capable of hydrolysing the peptide bonds between positions 214/215, and/or 323/324 (most preferred between position 214/215) in hVEGFR 2, or a peptide bond in proximity to these positions in hVEGFR 2, or peptide bonds in protein targets related to hVEGFR 2 at positions having structural homology or sequence homology to these positions.

In a thirty-eighth embodiment of this variant the enzyme is a protease and is capable of hydrolysing human Epidermal growth factor receptor (hEGFr). The enzymes or the fusion protein can thus be used for preparing medicaments for the treatment of disesaes, such as, but not limited to, bladder cancer, breast cancer, cervical cancer, colorectal cancer, endometrial cancer, oesophageal cancer, head and neck cancer, gastric cancer, non-small-cell lung carcinoma and ovarian cancer, as well as other diseases connected with hEGFr. Preferably, said enzyme or said fusion protein is capable of specifically inactivating hEGFr (SEQ ID NO: 116). More preferably said enzyme or said fusion protein is capable of hydrolysing the peptide bonds between positions 20/21, 29/30, 48/49, 74/75, 165/166, 202/203, 220/221, 246/247, 251/252, 269/270, 270/271, 304/305, 305/306, 357/358, 430/431, 443/444, 454/455, 455/456, 463/464, 465/466, 476/477, 507/508 and/or 509/510 in hEGFr, or a peptide bond in proximity to these positions in hEGFr, or peptide bonds in protein targets related to hEGFr at positions having structural homology or sequence homology to these positions.

In a thirty-ninth embodiment of this variant the enzyme is a protease and is capable of hydrolysing human Epithelial cell adhesion molecule (hEp-CAM). The enzymes or the fusion protein can thus be used for preparing medicaments for the treatment of diseases, such as, but not limited to, colorectal cancer, as well as other diseases connected with hEp-CAM. Preferably, said enzyme or said fusion protein is capable of specifically inactivating hEp-CAM (SEQ ID NO: 125). More preferably said enzyme or said fusion protein is capable of hydrolysing the peptide bonds between positions 14/15, 19/20, 25/26, 30/31, 33/34, 55/56 an/or 70/71 (most preferred between positions 14/15, 30/31 and/or 70/71) in hEp-CAM, or a peptide bond in proximity to these positions in hEp-CAM, or peptide bonds in protein targets related to hEp-CAM at positions having structural homology or sequence homology to these positions.

In a forty embodiment of this variant the enzyme is a protease and is capable of hydrolysing human Insulin-like growth factor I receptor (hIGF-1r). The enzymes or the fusion protein can thus be used for preparing medicaments for the treatment of diseases, such as, but not limited to, a variety of cancers including breast cancer, as well as other diseases connected with hIGF-1r. Preferably, said enzyme or said fusion protein is capable of specifically inactivating hIGF-1r (SEQ ID NO:126). More preferably said enzyme or said fusion protein is capable of hydrolysing the peptide bonds between positions 59/60, 115/116, 146/147, 171/172, 191/192, 290/291, 306/307, 307/308, 335/336, 336/337, 455/456 and/or 470/471 (most preferred between positions 306/307, 307/308, 335/336 and/or 470/471) in hIGF-1r, or a peptide bond in proximity to these positions in hIGF-1r, or peptide bonds in protein targets related to hIGF-1r at positions having structural homology or sequence homology to these positions.

In a forty-first embodiment of this variant the enzyme is a protease and is capable of hydrolysing human T-cell surface antigen CD2 precursor (hCD2). The enzymes or the fusion protein can thus be used for preparing medicaments for the treatment of diseases, such as, but not limited to, psoriasis, as well as other diseases connected with hCD2. Preferably, said enzyme or said fusion protein is capable of specifically inactivating hCD2 (SEQ ID NO:128). More preferably said enzyme or said fusion protein is capable of hydrolysing the peptide bonds between positions 42/43, 43/44, 48/49, 49/50, 51/52, 54/55, 63/64, 69/70, 89/90 and/or 91/92 (most preferred between positions 43/44, 51/52 and/or 89/90) in hCD2, or a peptide bond in proximity to these positions in hCD2, or peptide bonds in protein targets related to hCD2 at positions having structural homology or sequence homology to these positions.

In a forty-second embodiment of this variant the enzyme is a protease and is capable of hydrolysing human T-cell surface glycoprotein CD4 (hCD4). The enzymes or the fusion protein can thus be used for preparing medicaments for the treatment of diseases, such as, but not limited to, psoriasis, transplant rejection, graft-versus-host colitis, autoimmune disorders and rheumatoid arthritis, as well as other diseases connected with hCD4. Preferably, said enzyme or said fusion protein is capable of specifically inactivating hCD4 (SEQ ID NO:129). More preferably said enzyme or said fusion protein is capable of hydrolysing the peptide bonds between positions 166/167, 167/168, 206/207, 219/220, 224/225, 226/227, 251/252, 252/253, 322/323, 329/330 and/or 334/335 (most preferred between positions 206/207, 219/220, 251/252 and/or 252/253) in hCD4, or a peptide bond in proximity to these positions in hCD4, or peptide bonds in protein targets related to hCD4 at positions having structural homology or sequence homology to these positions.

In a forty-third embodiment of this variant the enzyme is a protease and is capable of hydrolysing human Integrin alpha-L (hCD1 la). The enzymes or the fusion protein can thus be used for preparing medicaments for the treatment of diseases, such as, but not limited to, psoriasis as well as other diseases connected with hCD11a. Preferably, said enzyme or said fusion protein is capable of specifically inactivating hCD11a (SEQ ID NO:130). More preferably said enzyme or said fusion protein is capable of hydrolysing the peptide bonds between positions 145/146, 152/153, 156/157, 159/160, 160/161, 177/178, 178/179, 189/190, 190/191, 191/192, 193/194, 197/198, 200/201, 221/222, 229/230, 249/250, 253/254, 268/269, 290/291, 297/298, 304/305 and/or 305/306 (most preferred between positions 145/146, 159/160, 160/161, 189/190, 229/230, 249/250, 268/269, 297/298, 304/305 and/or 305/306) in hCD11a, or a peptide bond in proximity to these positions in hCD11a, or peptide bonds in protein targets related to hCD11a at positions having structural homology or sequence homology to these positions.

In a forty-fourth embodiment of this variant the enzyme is a protease and is capable of hydrolysing human Interferon-gamma receptor alpha chain (hIFN-gamma-R1). The enzymes or the fusion protein can thus be used for preparing medicaments for the treatment of diseases, such as, but not limited to, classical Hodgkin's Lymphoma (cHL) and type I diabetes, as well as other diseases connected with hIFN-gamma-R1. Preferably, said enzyme or said fusion protein is capable of specifically inactivating hIFN-gamma-R1 (SEQ ID NO: 136). More preferably said enzyme or said fusion protein is capable of hydrolysing the peptide bonds between positions 49/50, 52/53, 62/63, 106/107, 122/123, 174/175, 215/216 and/or 222/223 (most preferred between positions 49/50, 122/123, 174/175 and/or 215/216) in hIFN-gamma-R1, or a peptide bond in proximity to these positions in hIFN-gamma-R1, or peptide bonds in protein targets related to hIFN-gamma-R1 at positions having structural homology or sequence homology to these positions.

In a forty-fifth embodiment of this variant the enzyme is a protease and is capable of hydrolysing human Platelet membrane glycoprotein IIb/IIIa (hGPIIb/IIIa). The enzymes or the fusion protein can thus be used for preparing medicaments for the treatment of diseases, such as, but not limited to, unstable angina, carotid stenting, ischemic stroke, peripheral vascular diseases, angiogenesis-related diseases and disseminating tumors, as well as other diseases connected with hGPIIb/IIIa. Preferably, said enzyme or said fusion protein is capable of specifically inactivating hGPIIb/IIIa (SEQ ID NO: 141). More preferably said enzyme or said fusion protein is capable of hydrolysing the peptide bonds between positions 67/68, 91/92, 129/130, 143/144, 144/145, 181/182, 208/209, 209/210, 216/217, 239/240, 261/262, 410/411, 532/533, 556/557, 557/558, 597/598, 650/651 and/or 689/690 (most preferred between positions 67/68, 261/262, 410/411, 650/651 and/or 689/690) in hGPIIb/IIIa, or a peptide bond in proximity to these positions in hGPIIb/IIIa, or peptide bonds in protein targets related to hGPIIb/IIIa at positions having structural homology or sequence homology to these positions.

In a forty-sixth embodiment of this variant the enzyme is a protease and is capable of hydrolysing human Intercellular adhesion molecule-1 (hICAM-1). The enzymes or the fusion protein can thus be used for preparing medicaments for the treatment of diseases, such as, but not limited to, Crohn's disease, as well as other diseases connected with hICAM-1. Preferably, said enzyme or said fusion protein is capable of specifically inactivating hICAM-1 (SEQ ID NO:142). More preferably said enzyme or said fusion protein is capable of hydrolysing the peptide bonds between positions 40/41, 88/89, 97/98, 102/103, 128/129, 131/132, 132/133, 149/150, 150/151, 160/161 and/or 166/167 (most preferred between positions 88/89, 102/103, 150/151, 160/161 and/or 166/167) in hICAM-1, or a peptide bond in proximity to these positions in hICAM-1, or peptide bonds in protein targets related to hICAM-1 at positions having structural homology or sequence homology to these positions.

In a forty-seventh embodiment of this variant the enzyme is a protease and is capable of hydrolysing human TGF-beta receptor type II (hTGF-beta RII). The enzymes or the fusion protein can thus be used for preparing medicaments for the treatment of diseases, such as, but not limited to, diffuse systemic sclerosis, as well as other diseases connected with hTGF-beta RII. Preferably, said enzyme or said fusion protein is capable of specifically inactivating hTGF-beta RII (SEQ ID NO: 145). More preferably said enzyme or said fusion protein is capable of hydrolysing the peptide bonds between positions 32/33, 34/35, 35/36, 66/67, 67/68, 69/70, 82/83, 103/104, 104/105, 105/106, 118/119, 122/123 and/or 130/131 (most preferred between positions 32/33, 34/35, 66/67, 69/70, 104/105, 122/123 and/or 130/131) in hTGF-beta RII, or a peptide bond in proximity to these positions in hTGF-beta RII, or peptide bonds in protein targets related to hTGF-beta RII at positions having structural homology or sequence homology to these positions.

In a forty-eighth embodiment of this variant the enzyme is a protease and is capable of hydrolysing human Membrane cofactor protein (hMCP). The enzymes or the fusion protein can thus be used for preparing medicaments for the treatment of diseases, such as, but not limited to, renal tumors, uveal melanomas and gastrointestinal tumors, as well as other diseases connected with hMCP. Preferably, said enzyme or said fusion protein is capable of specifically inactivating hMCP (SEQ ID NO: 146). More preferably said enzyme or said fusion protein is capable of hydrolysing the peptide bonds between positions 15/16, 17/18, 25/26, 31/32, 32/33, 35/36, 48/49, 67/68, 69/70, 110/111, 119/120 and/or 125/126 (most preferred between positions 15/16, 32/33, 48/49, 119/120 and/or 125/126) 130/131) in hMCP, or a peptide bond in proximity to these positions in hMCP, or peptide bonds in protein targets related to hMCP at positions having structural homology or sequence homology to these positions.

In a forty-ninth embodiment of this variant the enzyme is a protease and is capable of hydrolysing human Protease activated receptor 1 (hPAR1). The enzymes or the fusion protein can thus be used for preparing medicaments for the treatment of diseases, such as, but not limited to, thrombosis, as well as other diseases connected with hPAR1. Preferably, said enzyme or said fusion protein is capable of specifically inactivating hPAR1 (SEQ ID NO:110). More preferably said enzyme or said fusion protein is capable of hydrolysing the peptide bonds between positions 46/47, 51/52 and/or 52/53 in PAR1, or a peptide bond in proximity to these positions in hPAR1, or peptide bonds in protein targets related to hPAR1 at positions having structural homology or sequence homology to these positions.

In a fifth embodiment of this variant the enzyme is a protease and is capable of hydrolysing human Protease activated receptor 2 (hPAR2). The enzymes or the fusion protein can thus be used for preparing medicaments for the treatment of diseases, such as, but not limited to, Crohn's disease, Ulcerative colitis and Inflammatory bowel disease, asthma, inflammation associated pain and arthritis, as well as other diseases connected with hPAR2. Preferably, said enzyme or said fusion protein is capable of specifically inactivating hPAR2 (SEQ ID NO: 111). More preferably said enzyme or said fusion protein is capable of hydrolysing the peptide bonds between positions 41/42, 51/52 and/or 59/60 in hPAR2, or a peptide bond in proximity to these positions in hPAR2, or peptide bonds in protein targets related to hPAR2 at positions having structural homology or sequence homology to these positions.

In a fifty-first embodiment of this variant the enzyme is a protease and is capable of hydrolysing human Protease activated receptor 4 (hPAR4). The enzymes or the fusion protein can thus be used for preparing medicaments for the treatment of diseases, such as, but not limited to, thrombosis, as well as other diseases connected with hPAR4. Preferably, said enzyme or said fusion protein is capable of specifically inactivating hPAR4 (SEQ ID NO: 113). More preferably said enzyme or said fusion protein is capable of hydrolysing the peptide bonds between positions 68/69, 74/75 and/or 78/79 in hPAR4, or a peptide bond in proximity to these positions in hPAR4, or peptide bonds in protein targets related to hPAR4 at positions having structural homology or sequence homology to these positions.

In a fifty-second embodiment of this variant the enzyme is a protease and is capable of hydrolysing human 5-hydroxytryptamine 1A receptor (h5-HT-1A). The enzymes or the fusion protein can thus be used for preparing medicaments for the treatment of diseases, such as, but not limited to, irritable bowel syndrome, as well as other diseases connected with h5-HT-1A. Preferably, said enzyme or said fusion protein is capable of specifically inactivating h5-HT-1A (SEQ ID NO:117). More preferably said enzyme or said fusion protein is capable of hydrolysing the peptide bonds between positions 101/102, 102/103, 181/182 and/or 370/371 in h5-HT-1A a peptide bond in proximity to these positions in h5-HT-1A, or peptide bonds in protein targets related to h5-HT-1A at positions having structural homology or sequence homology to these positions.

In a fifty-third embodiment of this variant the enzyme is a protease and is capable of hydrolysing human carcinoembryonic antigen (hCEA). The enzymes or the fusion protein can thus be used for preparing medicaments for the treatment of diseases, such as, but not limited to, colon cancer, as well as other diseases connected with hCEA. Preferably, said enzyme or said fusion protein is capable of specifically inactivating hCEA (SEQ ID NO:138). More preferably said enzyme or said fusion protein is capable of hydrolysing the peptide bonds between positions 17/18, 69/70, 71/72, 74/75, 77/78, 98/99, 116/117, 126/127 and/or 128/129 in hCEA, or a peptide bond in proximity to these positions in hCEA, or peptide bonds in protein targets related to hCEA at positions having structural homology or sequence homology to these positions.

It is obvious to someone skilled in the art that also polymorphisms of all target sequences referred to are included. The expression “proximity to these positions” in all embodiments above refer to positions of peptide bonds that are between 10 and 5 Ångström and/or 5 amino acids, preferably 3 amino acids, next to the positions of the peptide bonds

Preferably, in this variant the scaffold of the engineered enzyme provided in step (c) is of human origin in order to avoid or reduce immunogenicity or allergenic effects associated with the application of the enzyme in the human body. Alternatively, immunoenicity and allergenicity can be reduced by deimmunization of the engineered enzyme.

In further embodiment of this variant, the target substrate is a pro-drug which is activated by the engineered enzyme. In a particular embodiment of this variant, the engineered enzyme has proteolytic activity and the target substrate is a protein target which is proteolytically activated. Examples of such pro-drugs are pro-proteins such as the inactivated forms of coagulations factors. In another particular variant, the engineered enzyme is an oxidoreductase and the target substrate is a chemical that can be activated by oxidation.

In a second variant of this aspect of the invention, the engineered enzyme is used for diagnostic puposes. In a particular embodiment of this variant, the engineered enzyme is target-specific protease. Such diagnostic purposes comprise but are not limited to applications with the aim of diagnosing diseases, testing genetic predispositions or monitoring disease progression during therapy.

In a particular embodiment, the diagnosis is based on the testing for the presence or absence of a disease-specific marker protein or a disease-specific variant of a human protein in test samples such as human tissue samples, blood samples or other samples taken from patients. The testing employs a protease with specificity for a particular, disease-related target protein. The testing is done by analysing the proteolytic degradation of such protein in the test sample.

In a preferred embodiment the aim of the diagnostic test is to detect and/or quantify a disease-specific variant of a native human protein. Such a diagnostic test employs a protease that is specific for the disease-related protein variant, i.e. it has significantly higher proteolytic activity on the disease-related protein variant compared to the native human protein. The disease-related protein variant is therefore detected and/or quantified by detecting and/or quantifying the activity of the target-specific protease. Such detection and/or quantification is done by directly measuring the degradation products of the target protein or indirectly by measuring the influence of the target protein on the activity of the target-specific protease by a competition assay. In another preferred embodiment the aim of the diagnostic test is to detect and/or quantify a protein that is specific for an infection by an infectious agent such as a virus or a bacterium. Such a diagnostic test employs a protease that is specific for a protein specifically expressed upon infection by the infectious agent, i.e. it has significantly higher proteolytic activity on a particular infection-indicating protein compared to any other native human protein. The infection-indicating protein is therefore detected and/or quantified by detecting and/or quantifying the proteolytic activity of the target-specific protease. Such detection and/or quantification is done by directly measuring the degradation products of the infection-indicating protein or indirectly by measuring the influence of the infection-indicating protein on the activity of the target-specific protease by a competition assay.

In a third variant of this aspect of the invention, the engineered enzyme is used as a technical means in order to catalyze an industrially or nutritionally relevant reaction with defined specificity. In a particular embodiment of this variant the engineered enzyme has proteolytic activity, the catalyzed reaction is a proteolytic processing, and the engineered enzyme specifically hydrolyses one or more industrially or nutrionally relevant protein substrates. In a preferred embodiment of this variant the engineered enzyme hydrolyses one or more industrially or nutrionally relevant protein substrates at specific sites, thereby leading to industrially or nutrionally desired product properties such as texture, taste or precipitation characteristics. In a further particular embodiment of this variant, the engineered enzyme catalyzes the hydrolysis of glycosidic bonds (glycosidase or glycosylases activity). Then, preferably, the catalyzed reaction is a polysaccharide processing, and the engineered enzyme specifically hydrolyses one or more industrially, technically or nutrionally relevant polysaccharide substrates. In a further particular embodiment of this variant, the engineered enzyme catalyzes the hydrolysis of triglyceride esters or lipids (lipase activity). Then, preferably, the catalyzed reaction is a lipid processing step, and the engineered enzyme specifically hydrolyses one or more industrially, technically or nutrionally relevant lipid substrates. In a further particular variant of this embodiment, the engineered enzyme catalyzes the oxidation or reduction of substrates (oxidoreductase activity). Then, preferably, the engineered enzyme specifically oxidizes or reduces one or more industrially, technically or nutrionally relevant chemical substrates.

A second aspect of the invention discloses engineered enzymes with defined specificities. These engineered enzymes are characterized by the following components:

(a) a protein scaffold capable of catalyzing at least one chemical reaction on a substrate, and

(b) one or more specificity determining regions (SDRs) located at sites in the protein scaffold that enable the resulting engineered protein to discriminate between ar least one target substrate and one or more different substrates, wherein the SDRs are essentially synthetic peptide sequences.

Preferably, such defined specificity of the engineered enzymes is not conferred by the protein scaffold.

In principle, the protein scaffold can have a variety of primary, secondary and tertiary structures. The primary structure, i.e. the amino acid sequence, can be an engineered sequence or can be derived from any viral, prokaryotic or eukaryotic origin. For human therapeutic use, however, the protein scaffold is preferably of mammalian origin, and more preferably, of human origin. Furthermore, the protein scaffold is capable to catalyze one or more chemical reactions and has preferably only a low specificity.

Preferably, derivatives of the protein scaffold are used that have modified amino acid sequences that confer improved characteristics for the applicability as protein scaffolds. Such improved characteristics comprise, but are not limited to, stability; expression or secretion yield; folding, in particular after combination of the protein scaffold with SDRs; increased or decreased sensitivity to regulators such as activators or inhibitors; immunogenicity; catalytic rate; kM or substrate affinity.

The engineered enzymes reveal their quantitative specificity from the synthetic peptide sequences that are combined with the protein scaffold. Therefore, the engineered peptide sequences are acting as Specificity Determining Regions or SDRs.

The number, the length and the positions of such SDRs can vary over a wide range. The number of SDRs within the scaffold is at least one, preferably more than one, more preferably between two and eleven, most preferably between two and six. The SDRs have a length between one and 50 amino acid residues, preferably a length between one and 15 amino acid residues, more preferably a length between one and six amino acid residues. Alternatively, the SDRs have a length between two and 20 amino acid residues, preferably a length between two and ten amino acid residues, more preferably a length between three and eight amino acid residues.

The inventive engineered enzymes can further be desribed as antibody-like protein molecules comprising constant and variable regions, but having a non-immunoglogulin backbone and having an active site (catalytic activity) in the constant region, whereby the substrate specificity of the active site is modulated by the variable region. Preferably, as in the immunoglobulin structure, the variable regions are loops of variable length and composition that interact with a target molecule.

In a particular variant of the invention, the engineered enzymes have hydrolase activity. In a preferred variant, the engineered enzymes have proteolytic activity. Particularly preferred protein scaffolds for this variant are unspecific proteases or are parts from unspecific proteases or are otherwise derived from unspecific proteases. The expressions “derived from” or “a derivative thereof” in this respect and in the following variants and embodiments refer to derivatives of proteins that are mutated at one or more amino acid positions and/or have a homology of at least 70%, preferably 90%, more preferably 95% and most preferably 99% to the original protein, and/or that are proteolytically processed, and/or that have an altered glycosylation pattern, and/or that are covalently linked to non-protein substances, and/or that are fused with further protein domains, and/or that have C-terminal and/or N-terminal truncations, and/or that have specific insertions, substitutions and/or deletions. Alternatively, “derived from” may refer to derivatives that are combinations or chimeras of two or more fragments from two or more proteins, each of which optionally comprises any or all of the aforementioned modifications. The tertiary structure of the protein scaffold can be of any type. Preferably, however, the tertiary structure belongs to one of the following structural classes: class S1 (chymotrypsin fold of the serine proteases family), class S8 (subtilisin fold of the serine proteases family), class SC (carboxypeptidase fold of the serine proteases family), class Al (pepsin A fold of the aspartic proteases), or class C14 (caspase-1 fold of the cysteine proteases). Examples of proteases that can serve as the protein scaffold of engineered proteolytic enzymes for the use as human therapeutics are or are derived from human trypsin, human thrombin, human chymotrypsin, human pepsin, human endothiapepsin, human caspases 1 to 14, and/or human furin.

The defined specificity of the engineered proteolytic enzymes is a measure of their ability to discriminate between at least one target peptide or protein substrates and one or more further peptide or protein substrates. Preferably, the defined specificity refers to the ability to discriminate peptide or protein substrates that differ in other positions than the PI site, more preferably, the defined specificity refers to the ability to discriminate peptide or protein substrates that differ in other positions than the P1 site and the P1′ site. Most preferably, the engineered proteolytic enzymes distinguish target peptid or protein substrates at as many sites as is necessary to preferentially hydrolyse the target substrate versus other proteins. As an example, a therapeutically useful engineered proteolytic enzyme applied intravenously in the human body should be sufficiently specific to discriminate between the target substrate and any other protein in the human serum. Preferably, such an engineered proteolytic enzyme recognizes and discriminates peptide substrates at three or more amino acid positions, more preferably at four or more positions, and even more preferably at five or more amino acid positions. These positions may either be adjacent or non-adjacent.

In a first embodiment , the protein scaffold has a tertiary structure or fold equal or similar to the tertiary structure or fold of the S1 structural subclass of serine proteases, i. e. the chymotrypsin fold, and/or has at least 70% identity on the amino acid level to a protein of the S1 structural subclass of serine proteases. It is preferred that SDRs are inserted into the protein scaffold at one or more positions from the group of positions that correspond structurally or by amino acid sequence homology to the regions 18-25, 38-48, 54-63, 73-86, 122-130, 148-156, 165-171 and 194-204 in human trypsin I, and more preferably at one or more positions from the group of positions that correspond structurally or by amino acid sequence homology to the regions 20-23, 41-45, 57-60, 76-83, 125-128, 150-153, 167-169 and 197-201 (numbering of amino acids according to SEQ ID NO: 1). The number of SDRs to be combined with this type of protein scaffold is preferably between 1 and 10, and more preferably between 2 and 4. Preferably, the protein scaffold is equal to or is a derivative or homologue of one or more of the following proteins: chymotrypsin, granzyme, kallikrein, trypsin, mesotrypsin, neutrophil elastase, pancreatic elastase, enteropeptidase, cathepsin, thrombin, ancrod, coagulation factor IXa, coagulation factor VIIa, coagulation factor Xa, activated protein C, urokinase, tissue-type plasminogen activator, plasmin, Desmodus-type plasminogen activator. More preferably, the protein scaffold is trypsin or thrombin or is a derivative or homologue from trypsin or thrombin. For the use as a human therapeutic, the trypsin or thrombin scaffold is most preferably of human origin in order to minimize the risk of an immune response or an allergenic reaction.

Preferably, derivatives with improved characteristics derived from human trypsin I or from proteins with similar tertiary structure are used. Preferred examples of such derivatives are derived from human trypsin I (SEQ ID NO:1) and comprise one or more of the following amino acid substitutions E56G; R78W; Y131F; A146T; C183R. It is preferred that at least one of two SDRs are inserted into human trypsin I, or a derivative thereof, between residues 42 and 43 (SDR 1) and between 123 and 124 (SDR 2), respectively (numbering of amino acids according to SEQ ID NO:1). In addition the SDR 1 has a preferred length of 6 and the SDR 2 has a preferred length of 5 amino acids, respectively. In a preferred variant of this embodiment, the SDR 1 and SDR 2 sequences comprise one of the amino acid sequences listed in table 2. Such engineered proteolytic enzymes have specificity for the target substrate B as exemplified in example IV.

In a further embodiment the protein scaffold belongs to the S8 structural subclass of serine proteases and/or has a tertiary structure similar to subtilisin E from Bacillus subtilis and/or has at least 70% identity on the amino acid level to a protein of the S8 structural subclass of serine proteases. Preferably, the scaffold belongs to the subtilisin family or the human pro-protein convertases. It is preferred that SDRs are inserted into the protein scaffold at one or more positions from the group of positions that correspond structurally or by amino acid sequence homology to the regions 6-17, 25-29, 47-55, 59-69, 101-111, 117-125, 129-137, 139-154, 158-169, 185-195 and 204-225 in subtilisin E from Bacillus subtilis, and more preferably at one or more positions from the group of positions that correspond structurally or by amino acid sequence homology to the regions 59-69, 101-111, 129-137, 158-169 and 204-225 (numbering of amino acids according to SEQ ID NO:7). It is preferred that the protein scaffold is equal to or is a derivative or homologue of one or more of the following proteins: subtilisin Carlsberg; B. subtilis subtilisin E; subtilisin BPN′; B. licheniformis subtilisin; B. lentus subtilisin; Bacillus alcalophilus alkaline protease; proteinase K; kexin; human pro-protein convertase; human furin. In a preferred variant, subtilisin BPN′ or one of the proteins SPC 1 to 7 is used as the protein scaffold.

In a further embodiment the protein scaffold belongs to the family of aspartic proteases and/or has a tertiary structure similar to human pepsin. Preferably, the scaffold belongs to the A1 class of proteases and/or has at least 70% identity on the amino acid level to a protein of the A1 class of proteases. It is preferred that SDRs are inserted into the protein scaffold at one or more positions from the group of positions that correspond structurally or by amino acid sequence homology to the regions 6-18, 49-55, 74-83, 91-97, 112-120, 126-137, 159-164, 184-194, 242-247, 262-267 and 277-300 in human pepsin, and more preferably at one or more positions from the group of positions that correspond structurally or by amino acid sequence homology to the regions 10-15, 75-80, 114-118, 130-134, 186-191 and 280-296 (numbering of amino acids according to SEQ ID NO:11). It is preferred that the protein scaffold is equal to or is a derivative or homologue of one or more of the following proteins: pepsin, chymosin, renin, cathepsin, yapsin. Preferably, pepsin or endothiopepsin or a derivative or homologue thereof is used as the protein scaffold.

In a further embodiment the protein scaffold belongs to the cysteine protease family and/or has a tertiary structure similar to human caspase 7. Preferably the scaffold belongs to the C14 class of cysteine proteases or has at least 70% identity on the amino acid level to a protein of the C14 class of cysteine proteases. It is preferred that SDRs are inserted into the protein scaffold at one or more positions from the group of positions that correspond structurally or by amino acid sequence homology to the regions 78-91, 144-160, 186-198, 226-243 and 271-291 in human caspase 7, and more preferably at one or more positions from the group of positions that correspond structurally or by amino acid sequence homology to the regions 80-86, 149-157, 190-194 and 233-238 (numbering of amino acids according to SEQ ID NO: 14). It is preferred that the protein scaffold is equal to or is a derivative or homologue of one of the caspases 1 to 9.

In a further embodiment the protein scaffold belongs to the S11 class of serine proteases or has at least 70% identity on the amino acid level to a protein of the S11 class of serine proteases and/or has a tertiary structure similar to D-alanyl-D-alanine transpeptidase from Streptomyces species K15. It is preferred that SDRs are inserted into the protein scaffold at one or more positions from the group of positions that correspond structurally or by amino acid sequence homology to the regions 67-79, 137-150, 191-206, 212-222 and 241-251 in D-alanyl-D-alanine transpeptidase from Streptomyces species K15, and more preferably at one or more positions from the group of positions that correspond structurally or by amino acid sequence homology to the regions 70-75, 141-147, 195-202 and 216-220 (numbering of amino acids according to SEQ ID NO: 15). It is preferred that the D-alanyl-D-alanine transpeptidase from Streptomyces species K15 or a derivative or homologue thereof is used as the scaffold.

In a further embodiment the protein scaffold belongs to the S21 class of serine proteases or has at least 70% identity on the amino acid level to a protein of the S21 class of serine proteases and/or has a tertiary structure similar to assemblin from human cytomegalovirus. It is preferred that SDRs are inserted into the protein scaffold at one or more positions from the group of positions that correspond structurally or by amino acid sequence homology to the regions 25-33, 64-69, 134-155, 162-169 and 217-244 in assemblin from human cytomegalovirus, and more preferably at one or more positions from the group of positions that correspond structurally or by amino acid sequence homology to the regions 27-31, 164-168 and 222-239 (numbering of amino acids according to SEQ ID NO:16). It is preferred that the assemblin from human cytomegalovirus or a derivative or homologue thereof is used as the scaffold.

In a further embodiment the protein scaffold belongs to the S26 class of serine proteases or has at least 70% identity on the amino acid level to a protein of the S26 class of serine proteases and/or has a tertiary structure similar to the signal peptidase from Escherichia coli. It is preferred that SDRs are inserted into the protein scaffold at one or more positions from the group of positions that correspond structurally or by amino acid sequence homology to the regions 8-14, 57-68, 125-134, 239-254, 200-211 and 228-239 in signal peptidase from Escherichia coli, and more preferably at one or more positions from the group of positions that correspond structurally or by amino acid sequence homology to the regions 9-13, 60-67, 127-132 and 203-209 (numbering of amino acids according to SEQ ID NO: 17). It is preferred that the signal peptidase from Escherichia coli or a derivative or homologue thereof is used as the scaffold.

In a further embodiment the protein scaffold belongs to the S33 class of serine proteases or has at least 70% identity on the amino acid level to a protein of the S33 class of serine proteases and/or has a tertiary structure similar to the prolyl aminopeptidase from Serratia marcescens. It is preferred that SDRs are inserted into the protein scaffold at one or more positions from the group of positions that correspond structurally or by amino acid sequence homology to the regions 47-54, 152-160, 203-212 and 297-302 in prolyl aminopeptidase from Serratia marcescens, and more preferably at one or more positions from the group of positions that correspond structurally or by amino acid sequence homology to the regions 50-53, 154-158 and 206-210 (numbering of amino acids according to SEQ ID NO:18). It is preferred that the prolyl aminopeptidase from Serratia marcescens or a derivative or homologue thereof is used as the scaffold.

In a further embodiment the protein scaffold belongs to the S51 class of serine proteases or has at least 70% identity on the amino acid level to a protein of the S51 class of serine proteases and/or has a tertiary structure similar to aspartyl dipeptidase from Escherichia coli. It is preferred that SDRs are inserted into the protein scaffold at one or more positions from the group of positions that correspond structurally or by amino acid sequence homology to the regions 8-16, 38-46, 85-92, 132-140, 159-170 and 205-211 in aspartyl dipeptidase from Escherichia coli, and more preferably at one or more positions from the group of positions that correspond structurally or by amino acid sequence homology to the regions 10-14, 87-90, 134-138 and 160-165 (numbering of amino acids according to SEQ ID NO:19). It is preferred that the aspartyl dipeptidase from Escherichia coli or a derivative or homologue thereof is used as the scaffold.

In a further embodiment the protein scaffold belongs to the A2 class of aspartic proteases or has at least 70% identity on the amino acid level to a protein of the A2 class of aspartic proteases and/or has a tertiary structure similar to the protease from human immunodeficiency virus. It is preferred that SDRs are inserted into the protein scaffold at one or more positions from the group of positions that correspond structurally or by amino acid sequence homology to the regions 5-12, 17-23, 27-30, 33-38 and 77-83 in protease from human immunodeficiency virus, and more preferably at one or more positions from the group of positions that correspond structurally or by amino acid sequence homology to the regions 7-10, 18-21, 34-37 and 79-82 (numbering of amino acids according to SEQ ID NO:20). It is preferred that the protease from human immunodeficiency virus, preferably HIV-1 protease, or a derivative or homologue thereof is used as the scaffold.

In a further embodiment the protein scaffold belongs to the A26 class of aspartic proteases or has at least 70% identity on the amino acid level to a protein of the A26 class of aspartic proteases and/or has a tertiary structure similar to the omptin from Escherichia coli. It is preferred that SDRs are inserted into the protein scaffold at one or more positions from the group of positions that correspond structurally or by amino acid sequence homology to the regions 28-40, 86-98, 150-168, 213-219 and 267-278 in omptin from Escherichia coli, and more preferably at one or more positions from the group of positions that correspond structurally or by amino acid sequence homology to the regions 33-38, 161-168 and 273-277 (numbering of amino acids according to SEQ ID NO:21). It is preferred that the omptin from Escherichia coli or a derivative or homologue thereof is used as the scaffold.

In a further embodiment the protein scaffold belongs to the C1 class of cysteine proteases or has at least 70% identity on the amino acid level to a protein of the C1 class of cysteine proteases and/or has a tertiary structure similar to the papain from Carica papaya. It is preferred that SDRs are inserted into the protein scaffold at one or more positions from the group of positions that correspond structurally or by amino acid sequence homology to the regions 17-24, 61-68, 88-95, 135-142, 153-158 and 176-184 in papain from Carica papaya, and more preferably at one or more positions from the group of positions that correspond structurally or by amino acid sequence homology to the regions 63-66, 136-139 and 177-181 (numbering of amino acids according to SEQ ID NO:22). It is preferred that the papain from Carica papaya or a derivative or homologue thereof is used as the scaffold.

In a further embodiment the protein scaffold belongs to the C2 class of cysteine proteases or has at least 70% identity on the amino acid level to a protein of the C2 class of cysteine proteases and/or has a tertiary structure similar to human calpain-2. It is preferred that SDRs are inserted into the protein scaffold at one or more positions from the group of positions that correspond structurally or by amino acid sequence homology to the regions 90-103, 160-172, 193-199, 243-260, 286-294 and 316-322 in human calpain-2, and more preferably at one or more positions from the group of positions that correspond structurally or by amino acid sequence homology to the regions 92-101, 245-250 and 287-291 (numbering of amino acids according to SEQ ID NO:23). It is preferred that the human calpain-2 or a derivative or homologue thereof is used as the scaffold.

In a further embodiment the protein scaffold belongs to the C4 class of cysteine proteases or has at least 70% identity on the amino acid level to a protein of the C4 class of cysteine proteases and/or has a tertiary structure similar to NIa protease from tobacco etch virus. It is preferred that SDRs are inserted into the protein scaffold at one or more positions from the group of positions that correspond structurally or by amino acid sequence homology to the regions 23-31, 112-120, 144-150, 168-176 and 205-218 in NIa protease from tobacco etch virus, and more preferably at one or more positions from the group of positions that correspond structurally or by amino acid sequence homology to the regions 145-149, 169-174 and 212-218 (numbering of amino acids according to SEQ ID NO:24). It is preferred that the NIa protease from tobacco etch virus (TEV protease) or a derivative or homologue thereof is used as the scaffold.

In a further embodiment the protein scaffold belongs to the C10 class of cysteine proteases or has at least 70% identity on the amino acid level to a protein of the C10 class of cysteine proteases and/or has a tertiary structure similar to the streptopain from Streptococcus pyogenes. It is preferred that SDRs are inserted into the protein scaffold at one or more positions from the group of positions that correspond structurally or by amino acid sequence homology to the regions 81-90, 133-140, 150-164, 191-199, 219-229, 246-256, 306-312 and 330-337 in streptopain from Streptococcus pyogenes, and more preferably at one or more positions from the group of positions that correspond structurally or by amino acid sequence homology to the regions 82-87, 134-138, 250-254 and 331-335 (numbering of amino acids according to SEQ ID NO:25). It is preferred that the streptopain from Streptococcus pyogenes or a derivative or homologue thereof is used as the scaffold.

In a further embodiment the protein scaffold belongs to the C19 class of cysteine proteases or has at least 70% identity on the amino acid level to a protein of the C1 9 class of cysteine proteases and/or has a tertiary structure similar to human ubiquitin specific protease 7. It is preferred that SDRs are inserted into the protein scaffold at one or more positions from the group of positions that correspond structurally or by amino acid sequence homology to the regions 3-15, 63-70, 80-86, 248-256, 272-283 and 292-304 in human ubiquitin specific protease 7, and more preferably at one or more positions from the group of positions that correspond structurally or by amino acid sequence homology to the regions 10-15, 251-255, 277-281 and 298-304 (numbering of amino acids according to SEQ ID NO:26). It is preferred that the human ubiquitin specific protease 7 or a derivative or homologue thereof is used as the scaffold.

In a further embodiment the protein scaffold belongs to the C47 class of cysteine proteases or has at least 70% identity on the amino acid level to a protein of the C47 class of cysteine proteases and/or has a tertiary structure similar to the staphopain from Staphylococcus aureus. It is preferred that SDRs are inserted into the protein scaffold at one or more positions from the group of positions that correspond structurally or by amino acid sequence homology to the regions 15-23, 57-66, 108-119, 142-149 and 157-164 in staphopain from Staphylococcus aureus, and more preferably at one or more positions from the group of positions that correspond structurally or by amino acid sequence homology to the regions 17-22, 111-117, 143-147 and 159-163 (numbering of amino acids according to SEQ ID NO:27). It is preferred that the staphopain from Staphylococcus aureus or a derivative or homologue thereof is used as the scaffold.

In a further embodiment the protein scaffold belongs to the C48 class of cysteine proteases or has at least 70% identity on the amino acid level to a protein of the C48 class of cysteine proteases and/or has a tertiary structure similar to the U1p1 endopeptidase from Saccharomyces cerevisiae. It is preferred that SDRs are inserted into the protein scaffold at one or more positions from the group of positions that correspond structurally or by amino acid sequence homology to the regions 40-51, 108-115, 132-141, 173-179 and 597-605 in U1p1 endopeptidase from Saccharomyces cerevisiae, and more preferably at one or more positions from the group of positions that correspond structurally or by amino acid sequence homology to the regions 43-49, 110-113, 133-137 and 175-178 (numbering of amino acids according to SEQ ID NO:28). It is preferred that the U1p1 endopeptidase from Saccharomyces cerevisiae or a derivative or homologue thereof is used as the scaffold.

In a further embodiment the protein scaffold belongs to the C56 class of cysteine proteases or has at least 70% identity on the amino acid level to a protein of the C56 class of cysteine proteases and/or has a tertiary structure similar to the Pfp1endopeptidase from Pyrococcus horikoshii. It is preferred that SDRs are inserted into the protein scaffold at one or more positions from the group of positions that correspond structurally or by amino acid sequence homology to the regions 8-16, 40-47, 66-73, 118-125 and 147-153 in Pfp1 endopeptidase from Pyrococcus horikoshii, and more preferably at one or more positions from the group of positions that correspond structurally or by amino acid sequence homology to the regions 9-14, 68-71, 120-123 and 148-151 (numbering of amino acids according to SEQ ID NO:29). It is preferred that the Pfp1 endopeptidase from Pyrococcus horikoshii or a derivative or homologue thereof is used as the scaffold.

In a further embodiment the protein scaffold belongs to the M4 class of metallo proteases or has at least 70% identity on the amino acid level to a protein of the M4 class of metallo proteases and/or has a tertiary structure similar to thermolysin from Bacillus thermoproteolyticus. It is preferred that SDRs are inserted into the protein scaffold at one or more positions from the group of positions that correspond structurally or by amino acid sequence homology to the regions 106-118, 125-130, 152-160, 197-204, 210-213 and 221-229 in thermolysin from Bacillus thermoproteolyticus, and more preferably at one or more positions from the group of positions that correspond structurally or by amino acid sequence homology to the regions 108-115, 126-129, 199-203 and 223-227 (numbering of amino acids according to SEQ ID NO:30). It is preferred that the thermolysin from Bacillus thermoproteolyticus or a derivative or homologue thereof is used as the scaffold.

In a further embodiment the protein scaffold belongs to the M10 class of metallo proteases or has at least 70% identity on the amino acid level to a protein of the M10 class of metallo proteases and/or has a tertiary structure similar to human collagenase. It is preferred that SDRs are inserted into the protein scaffold at one or more positions from the group of positions that correspond structurally or by amino acid sequence homology to the regions 2-7, 68-79, 85-90, 107-111 and 135-141 in human collagenase, and more preferably at one or more positions from the group of positions that correspond structurally or by amino acid sequence homology to the regions 3-6, 71-78 and 136-140 (numbering of amino acids according to SEQ ID NO:31). It is preferred that human collagenase or a derivative or homologue thereof is used as the scaffold.

It is further preferred that the engineered enzymes have glycosidase activity. A particularly suited protein scaffold for this variant is a glycosylase or is derived from a glycosylase. Preferably, the tertiary structure belongs to one of the following structural classes: class GH13, GH7, GH12, GH11, GH11, GH28, GH26, and GH18 (beta/alpha)8 barrel.

In a first embodiment the protein scaffold belongs to the GH13 class of glycosylases or has at least 70% identity on the amino acid level to a protein of the GH13 class of glycosylases and/or has a tertiary structure similar to human pancreatic alpha-amylase. It is preferred that SDRs are inserted into the protein scaffold at one or more positions from the group of positions that correspond structurally or by amino acid sequence homology to the regions 50-60, 100-110, 148-167, 235-244, 302-310 and 346-359 in human pancreatic alpha-amylase, and more preferably at one or more positions from the group of positions that correspond structurally or by amino acid sequence homology to the regions 51-58, 148-155 and 303-309 (numbering of amino acids according to SEQ ID NO:32). It is preferred that human pancreatic alpha-amylase or a derivative or homologue thereof is used as the scaffold.

In a further embodiment the protein scaffold belongs to the GH7 class of glycosylases or has at least 70% identity on the amino acid level to a protein of the GH7 class of glycosylases and/or has a tertiary structure similar to cellulase from Trichoderma reesei. It is preferred that SDRs are inserted into the protein scaffold at one or more positions from the group of positions that correspond structurally or by amino acid sequence homology to the regions 47-56, 93-104, 173-182, 215-223, 229-236 and 322-334 in cellulase from Trichoderma reesei, and more preferably at one or more positions from the group of positions that correspond structurally or by amino acid sequence homology to the regions 175-180, 218-222 and 324-332 (numbering of amino acids according to SEQ ID NO:33). It is preferred that cellulase from Trichoderma reesei or a derivative or homologue thereof is used as the scaffold.

In a further embodiment the protein scaffold belongs to the GH12 class of glycosylases or has at least 70% identity on the amino acid level to a protein of the GH12 class of glycosylases and/or has a tertiary structure similar to cellulase from Aspergillus niger. It is preferred that SDRs are inserted into the protein scaffold at one or more positions from the group of positions that correspond structurally or by amino acid sequence homology to the regions 18-28, 55-60, 106-113, 126-132 and 149-159 in cellulase from Aspergillus niger, and more preferably at one or more positions from the group of positions that correspond structurally or by amino acid sequence homology to the regions 20-26, 56-59, 108-112 and 151-156 (numbering of amino acids according to SEQ ID NO:34). It is preferred that cellulase from Aspergillus niger or a derivative or homologue thereof is used as the scaffold.

In a further embodiment the protein scaffold belongs to the GH11 class of glycosylases or has at least 70% identity on the amino acid level to a protein of the GH11 class of glycosylases and/or has a tertiary structure similar to xylanase from Aspergillus niger. It is preferred that SDRs are inserted into the protein scaffold at one or more positions from the group of positions that correspond structurally or by amino acid sequence homology to the regions 7-14, 33-39, 88-97, 114-126 and 158-167 in xylanase from Aspergillus niger, and more preferably at one or more positions from the group of positions that correspond structurally or by amino acid sequence homology to the regions 20-26, 56-59, 108-112 and 151-156 (numbering of amino acids according to SEQ ID NO:35). It is preferred that xylanase from Aspergillus niger or a derivative or homologue thereof is used as the scaffold.

In a further embodiment the protein scaffold belongs to the GH10 class of glycosylases or has at least 70% identity on the amino acid level to a protein of the GH10 class of glycosylases and/or has a tertiary structure similar to xylanase from Streptomyces lividans. It is preferred that SDRs are inserted into the protein scaffold at one or more positions from the group of positions that correspond structurally or by amino acid sequence homology to the regions 21-29, 42-50, 84-92, 130-136, 206-217 and 269-278 in xylanase from Streptomyces lividans, and more preferably at one or more positions from the group of positions that correspond structurally or by amino acid sequence homology to the regions 43-49, 86-90, 208-213 and 271-276 (numbering of amino acids according to SEQ ID NO:36). It is preferred that xylanase from Streptomyces lividans or a derivative or homologue thereof is used as the scaffold.

In a further embodiment the protein scaffold belongs to the GH28 class of glycosylases or has at least 70% identity on the amino acid level to a protein of the GH28 class of glycosylases and/or has a tertiary structure similar to pectinase from Aspergillus niger. It is preferred that SDRs are inserted into the protein scaffold at one or more positions from the group of positions that correspond structurally or by amino acid sequence homology to the regions 82-88, 118-126, 171-178, 228-236, 256-264 and 289-299 in pectinase from Aspergillus niger, and more preferably at one or more positions from the group of positions that correspond structurally or by amino acid sequence homology to the regions 116-124, 174-178 and 291-296 (numbering of amino acids according to SEQ ID NO:37). It is preferred that pectinase from Aspergillus niger or a derivative or homologue thereof is used as the scaffold.

In a further embodiment the protein scaffold belongs to the GH26 class of glycosylases or has at least 70% identity on the amino acid level to a protein of the GH26 class of glycosylases and/or has a tertiary structure similar to mannanase from Pseudomonas cellulosa. It is preferred that SDRs are inserted into the protein scaffold at one or more positions from the group of positions that correspond structurally or by amino acid sequence homology to the regions 75-83, 113-125, 174-182, 217-224, 247-254, 324-332 and 325-340 in mannanase from Pseudomonas cellulosa, and more preferably at one or more positions from the group of positions that correspond structurally or by amino acid sequence homology to the regions 115-123, 176-180, 286-291 and 328-337 (numbering of amino acids according to SEQ ID NO:38). It is preferred that mannanase from Pseudomonas cellulosa or a derivative or homologue thereof is used as the scaffold.

In an further embodiment the protein scaffold belongs to the GH18 (beta/alpha)8 barrel class of glycosylases or has at least 70% identity on the amino acid level to a protein of the GH18 class of glycosylases and/or has a tertiary structure similar to chitinase from Bacillus circulans. It is preferred that SDRs are inserted into the protein scaffold at one or more positions from the group of positions that correspond structurally or by amino acid sequence homology to the regions 21-29, 57-65, 130-136, 176-183, 221-229, 249-257 and 327-337 in chitinase from Bacillus circulans, and more preferably at one or more positions from the group of positions that correspond structurally or by amino acid sequence homology to the regions 59-63, 178-181, 250-254 and 330-336 (numbering of amino acids according to SEQ ID NO:39). It is preferred that chitinase from Bacillus circulans or a derivative or homologue thereof is used as the scaffold.

It is further preferred that the engineered enzymes have esterhydrolase activity. Preferably, the protein scaffold for this variant have lipase, phosphatase, phytase, or phosphodiesterase activity.

In a first embodiment the protein scaffold belongs to the GX class of esterases or has at least 70% identity on the amino acid level to a protein of the GX class of esterases and/or has a tertiary structure similar to the structure of the lipase B from Candida antarctica. Preferably, the scaffold has lipase activity. It is preferred that SDRs are inserted into the protein scaffold at one or more positions from the group of positions that correspond structurally or by amino acid sequence homology to the regions 139-148, 188-195, 216-224, 256-266, 272-287 in lipase B from Candida antarctica, and more preferably at one or more positions from the group of positions that correspond structurally or by amino acid sequence homology to the regions 141-146, 218-222, 259-263 and 275-283 (numbering of amino acids according to SEQ ID NO:40). It is preferred that lipase B from Candida antarctica or a derivative or homologue thereof is used as the scaffold.

In a further embodiment the protein scaffold belongs to the GX class of esterases or has at least 70% identity on the amino acid level to a protein of the GX class of esterases and/or has a tertiary structure similar to the pancreatic lipase from guinea pig. Preferably, the scaffold has lipase activity. It is preferred that SDRs are inserted into the protein scaffold at one or more positions from the group of positions that correspond structurally or by amino acid sequence homology to the regions 78-90, 91-100, 112-120, 179-186, 207-218, 238-247 and 248-260 in pancreatic lipase from guinea pig, and more preferably at one or more positions from the group of positions that correspond structurally or by amino acid sequence homology to the regions 80-87, 114-118, 209-215 and 239-246 (numbering of amino acids according to SEQ ID NO:41). It is preferred that pancreatic lipase from guinea pig or a derivative or homologue thereof is used as the scaffold.

In a further embodiment the protein scaffold has a tertiary structure similar to the structure of the alkaline phosphatase from Escherichia coli or has at least 70% identity on the amino acid level to a protein that has a tertiary structure similar to the structure of the alkaline phosphatase from Escherichia coli. Preferably, the scaffold has phosphatase activity. It is preferred that SDRs are inserted into the protein scaffold at one or more positions from the group of positions that correspond structurally or by amino acid sequence homology to the regions 110-122, 187-142, 170-175, 186-193, 280-287 and 425-435 in alkaline phosphatase from Escherichia coli, and more preferably at one or more positions from the group of positions that correspond structurally or by amino acid sequence homology to the regions 171 -174, 187-191, 282-286 and 426-433 (numbering of amino acids according to SEQ ID NO:42). It is preferred that alkaline phosphatase from Escherichia coli or a derivative or homologue thereof is used as the scaffold.

In a further embodiment the protein scaffold has a tertiary structure similar to the structure of the bovine pancreatic desoxyribonuclease I or has at least 70% identity on the amino acid level to a protein that has a tertiary structure similar to the structure of the bovine pancreatic desoxyribonuclease I. Preferably, the scaffold has phosphodiesterase activity. More preferably, a nuclease, and most preferably, an unspecific endonuclease or a derivative thereof is used as the scaffold. It is preferred that SDRs are inserted into the protein scaffold at one or more positions from the group of positions that correspond structurally or by amino acid sequence homology to the regions 14-21, 41-47, 72-77, 97-111, 135-143, 171-178, 202-209 and 242-251 in bovine pancreatic desoxyribonuclease I, and more preferably at one or more positions from the group of positions that correspond structurally or by amino acid sequence homology to the regions 16-19, 42-46, 136-141 and 172-176 (numbering of amino acids according to SEQ ID NO:43). It is preferred that bovine pancreatic desoxyribonuclease I or human desoxyribonuclease I or a derivative or homologue thereof is used as the scaffold.

It is further preferred that the engineered enzyme has transferase activity. A particularly suited protein scaffold for this variant is a glycosyl-, a phospho- or a methyltransferase, or is a derivative thereof. Particularly preferred protein scaffolds for this variant are glycosyltransferases or are derived from glycosyltransferases. The tertiary structure of the protein scaffold can be of any type. Preferably, however, the tertiary structure belongs to one of the following structural classes: GH13 and GT1.

In a first embodiment the protein scaffold belongs to the GH13 class of transferases or has at least 70% identity on the amino acid level to a protein of the GH13 class of transferases and/or has a tertiary structure similar to the structure of the cyclomaltodextrin glucanotransferase from Bacillus circulans. Preferably, the scaffold has transferase activity, and more preferably a glycosyltransferase is used as the scaffold. It is preferred that SDRs are inserted into the protein scaffold at one or more positions from the group of positions that correspond structurally or by amino acid sequence homology to the regions 38-48, 85-94, 142-154, 178-186, 259-266, 331-340 and 367-377 in cyclomaltodextrin glucanotransferase from Bacillus circulans, and more preferably at one or more positions from the group of positions that correspond structurally or by amino acid sequence homology to the regions 87-92, 180-185, 261-264 and 269-275 (numbering of amino acids according to SEQ ID NO:44). It is preferred that cyclomaltodextrin glucanotransferase from Bacillus circulans or a derivative or homologue thereof is used as the scaffold.

In a further embodiment the protein scaffold belongs to the GT1 class of tranferases or has at least 70% identity on the amino acid level to a protein of the GT1 class of transferases and/or has a tertiary structure similar to the structure of the glycosyltransferase from Amycolatopsis orientalis A82846. Preferably the scaffold has transferase activity, and more preferably glycosyltransferase activity. It is preferred that SDRs are inserted into the protein scaffold at one or more positions from the group of positions that correspond structurally or by amino acid sequence homology to the regions 58-74, 130-138, 185-193, 228-236 and 314-323 in glycosyltransferase from Amycolatopsis orientalis A82846, and more preferably at one or more positions from the group of positions that correspond structurally or by amino acid sequence homology to the regions 61-71, 230-234 and 316-321 (numbering of amino acids according to SEQ ID NO:45). It is preferred that the glycosyltransferase from Amycolatopsis orientalis A82846 or a derivative or homologue thereof is used as the scaffold.

It is further preferred that the engineered enzymes have oxidoreductase activity. A particularly suited protein scaffold for this variant is a monooxygenase, a dioxygenase or a alcohol dehydrogenase, or a derivative thereof. The tertiary structure of the protein scaffold can be of any type.

In a first embodiment the protein scaffold has a tertiary structure similar to the structure of the 2,3-diphydroxybiphenyl dioxygenase from Pseudomonas sp. or has at least 70% identity on the amino acid level to a protein that has a tertiary structure similar to the structure of the 2,3-diphydroxybiphenyl dioxygenase from Pseudomonas sp. Preferably, the scaffold has dioxygenase activity. It is preferred that SDRs are inserted into the protein scaffold at one or more positions from the group of positions that correspond structurally or by amino acid sequence homology to the regions 172-185, 198-206, 231-237, 250-259 and 282-287 in 2,3-diphydroxybiphenyl dioxygenase from Pseudomonas sp., and more preferably at one or more positions from the group of positions that correspond structurally or by amino acid sequence homology to the regions 175-182, 200-204, 252-257 and 284-287 (numbering of amino acids according to SEQ ID NO:46). It is preferred that the 2,3-diphydroxybiphenyl dioxygenase from Pseudomonas sp or a derivative or homologue thereof is used as the scaffold.

In a further embodiment the protein scaffold has a tertiary structure similar to the structure of the catechol dioxygenase from Acinetobacter sp. or has at least 70% identity on the amino acid level to a protein that has a tertiary structure similar to the structure of the catechol dioxygenase from Acinetobacter sp.. Preferably, the scaffold has dioxygenase activity, and more preferably catechol dioxygenase activity. It is preferred that SDRs are inserted into the protein scaffold at one or more positions from the group of positions that correspond structurally or by amino acid sequence homology to the regions 66-72, 105-112, 156-171 and 198-207 in catechol dioxygenase from Acinetobacter sp., and more preferably at one or more positions from the group of positions that correspond structurally or by amino acid sequence homology to the regions 107-110, 161-171 and 201-205 (numbering of amino acids according to SEQ ID NO:47). It is preferred that the catechol dioxygenase from Acinetobacter sp or a derivative or homologue thereof is used as the scaffold.

In a further embodiment the protein scaffold has a tertiary structure similar to the structure of the camphor-5-monooxygenase from Pseudomonas putida or has at least 70% identity on the amino acid level to a protein that has a tertiary structure similar to the structure of the camphor-5-monooxygenase from Pseudomonas putida. Preferably, the scaffold has monooxygenase activity, and more preferably camphor monooxygenase activity. It is preferred that SDRs are inserted into the protein scaffold at one or more positions from the group of positions that correspond structurally or by amino acid sequence homology to the regions 26-31, 57-63, 84-98, 182-191, 242-256, 292-299 and 392-399 in camphor-5-monooxygenase from Pseudomonas putida, and more preferably at one or more positions from the group of positions that correspond structurally or by amino acid sequence homology to the regions 85-96, 183-188, 244-253, 293-298 and 393-398 (numbering of amino acids according to SEQ ID NO:48). It is preferred that the camphor-5-monooxygenase from Pseudomonas putida or a derivative or homologue thereof is used as the scaffold.

In a further embodiment the protein scaffold has a tertiary structure similar to the structure of the alcohol dehydrogenase from Equus callabus or has at least 70% identity on the amino acid level to a protein that has a tertiary structure similar to the structure of the alcohol dehydrogenase from Equus callabus. Preferably, the scaffold has alcohol dehydrogenase activity. It is preferred that SDRs are inserted into the protein scaffold at one or more positions from the group of positions that correspond structurally or by amino acid sequence homology to the regions 49-63, 111-112, 294-301 and 361-369 in alcohol dehydrogenase from Equus callabus, and more preferably at one or more positions from the group of positions that correspond structurally or by amino acid sequence homology to the regions 51-61 and 295-299 (numbering of amino acids according to SEQ ID NO:49). It is preferred that the alcohol dehydrogenase from Equus callabus or a derivative or homologue thereof is used as the scaffold.

It is further preferred that the engineered enzymes have lyase activity. A particularly suited protein scaffold for this variant is a oxoacid lyase or is a derivative thereof. Particularly preferred protein scaffolds for this variant are aldolases or synthases, or are derived thereof. The tertiary structure of the protein scaffold can be of any type, but a (beta/alpha)8 barrel structure is preferred.

In a first embodiment the protein scaffold has a tertiary structure similar to the structure of the N-acetyl-d-neuramic acid aldolase from Escherichia coli or has at least 70% identity on the amino acid level to a protein that has a tertiary structure similar to the structure of the N-acetyl-d-neuramic acid aldolase from Escherichia coli. Preferably, the scaffold has aldolase activity. It is preferred that SDRs are inserted into the protein scaffold at one or more positions from the group of positions that correspond structurally or by amino acid sequence homology to the regions 45-55, 78-87, 105-113, 137-146, 164-171, 187-193, 205-210, 244-255 and 269-276 in N-acetyl-d-neuramic acid aldolase from Escherichia coli, and more preferably at one or more positions from the group of positions that correspond structurally or by amino acid sequence homology to the regions 45-52, 138-144, 189-192, 247-253 and 271-275 (numbering of amino acids according to SEQ ID NO:50). It is preferred that the N-acetyl-d-neuramic acid aldolase from Escherichia coli or a derivative or homologue thereof is used as the scaffold.

In a further embodiment the protein scaffold has a tertiary structure similar to the structure of the tryptophan synthase from Salmonella typhimurium or has at least 70% identity on the amino acid level to a protein that has a tertiary structure similar to the structure of the tryptophan synthase from Salmonella typhimurium. Preferably, the scaffold has synthase activity. It is preferred that SDRs are inserted into the protein scaffold at one or more positions from the group of positions that correspond structurally or by amino acid sequence homology to the regions 56-63, 127-134, 154-161, 175-193, 209-216 and 230-240 in tryptophan synthase from Salmonella typhimurium, and more preferably at one or more positions from the group of positions that correspond structurally or by amino acid sequence homology to the regions 57-62, 155-160, 178-190 and 210-215 (numbering of amino acids according to SEQ ID NO:51). It is preferred that the tryptophan synthase from Salmonella typhimurium or a derivative or homologue thereof is used as the scaffold.

It is further preferred that the engineered enzymes have isomerase activity. A particularly suited protein scaffold for this variant is a converting aldose or a converting ketose, or is a derivative thereof.

In a first embodiment, the protein scaffold has a tertiary structure similar to the structure of the xylose isomerase from Actinoplanes missouriensis or has at least 70% identity on the amino acid level to a protein that has a tertiary structure similar to the structure of the xylose isomerase from Actinoplanes missouriensis. It is preferred that SDRs are inserted into the protein scaffold at one or more positions from the group of positions that correspond structurally or by amino acid sequence homology to the regions 18-31, 92-103, 136-147, 178-188 and 250-257 in xylose isomerase from Actinoplanes missouriensis, and more preferably at one or more positions from the group of positions that correspond structurally or by amino acid sequence homology to the regions 20-27, 92-99 and 180-186 (numbering of amino acids according to SEQ ID NO:52). It is preferred that the xylose isomerase from Actinoplanes missouriensis or a derivative or homologue thereof is used as the scaffold.

It is further preferred that the engineered enzymes have ligase activity. A particularly suited protein scaffold for this variant is a DNA ligase, or is a derivative thereof.

In a first embodiment, the protein scaffold has a tertiary structure similar to the structure of the DNA ligase from Bacteriophage T7 or has at least 70% identity on the amino acid level to a protein that has a tertiary structure similar to the structure of the DNA-ligase from Bacteriophage T7. It is preferred that SDRs are inserted into the protein scaffold at one or more positions from the group of positions that correspond structurally or by amino acid sequence homology to the regions 52-60, 94-108, 119-131, 241-248, 255-263 and 302-318 in DNA ligase from Bacteriophage T7, and more preferably at one or more positions from the group of positions that correspond structurally or by amino acid sequence homology to the regions 96-106, 121-129, 256-262 and 304-316 (numbering of amino acids according to SEQ ID NO:53). It is preferred that the DNA ligase from Bacteriophage T7 or a derivative or homologue thereof is used as the scaffold.

A third aspect of the invention is directed to a method for generating engineered enzymes with specificities that are qualitatively and/or quantitatively novel in combination with the protein scaffold. The inventive method comprises at least the following steps:

(a) providing a protein scaffold capable to catalyze at least one chemical reaction on at least one target substrate,

(b) generating a library of engineered enzymes or isolated engineered enzymes by combining the protein scaffold from step (a) with one or more fully or partially random peptide sequences at sites in the protein scaffold that enable the resulting engineered enzyme to discriminate between at least one target substrate and one or more different substrates and

(c) selecting out of the library of engineered enzymes generated in step (b) one or more enzymes that have defined specificities towards at least one target substrate.

In a first variant of this aspect of the invention, the inventive method comprises at least the following steps:

(a) providing a protein scaffold capable to catalyze at least one chemical reaction on at least one target substrate,

(b) generating a library of engineered enzymes or isolated engineered enzymes by inserting into the protein scaffold from step (a) one or more fully or partially random peptide sequences at sites in the protein scaffold that enable the resulting engineered enzyme to discriminate between at least one target substrate and one or more different substrates and

(c) selecting out of the library of engineered enzymes generated in step (b) one or more enzymes that have defined specificities towards at least one target substrate.

Preferably, the positions at which the one or more fully or partially random peptide sequences are combined with or inserted into the protein scaffold are identified prior to the combination or insertion.

The number of insertions or other combinations of fully or partially random peptide sequences as well as their length may vary over a wide range. The number is at least one, preferably more than one, more preferably between two and eleven, most preferably between two and six. The length of such fully or partially random peptide sequences is usually less than 50 amino acid residues. Preferably, the length is between one and 15 amino acid residues, more preferably between one and six amino acid residues. Alternatively, the length is between two and 20 amino acid residues, preferably between two and ten amino acid residues, more preferably between three and eight amino acid residues.

Preferably such insertions or other combinations are performed on the DNA level, using polynucleotides encoding such protein scaffolds and polynucleotides or oligonucleotides encoding such fully or partially random peptide sequences.

Optionally, steps (a) to (c) are repeated cyclically, whereby enzymes selected in step (c) serve as the protein scaffold in step (a) of a further cycle, and randomized peptide sequences are either inserted or, alternatively, substituted for peptide sequences that have been inserted in former cycles. Thereby, the number of inserted peptide sequences is either constant or increases over the cycles. The cycles are repeated until one or more enzymes with the intended specificities are generated.

Moreover, during or after one or more rounds of steps (a) to (c), the scaffold may be mutated at one or more positions in order to make the scaffold more acceptable for the combination with SDR sequences, and/or to increase catalytic activity at a specific pH and temperature, and/or to change the glycosylation pattern, and/or to decrease sensitivity towards enzyme inhibitors, and/or to change enzyme stability.

In a second variant of this aspect of the invention, the inventive method comprises at least the following steps:

(a) providing a first protein scaffold fragment,

(b) connecting said protein scaffold fragment via a peptide linkage with a first SDR, and optionally

(c) connecting the product of step (b) via a peptide linkage with a further SDR peptide or with a further protein scaffold fragment, and optionally

(d) repeating step (c) for as many cycles as necessary in order to generate a sufficiently specific enzyme, and

(e) selecting out of the population generated in steps (a) - (d) one or more enzymes that have the desired specificities toward the one or more target substrates.

Protein scaffold fragment means a part of the sequence of a protein scaffold. A protein scaffold is comprised of at least two protein scaffold fragments.

In a third variant of this aspect of the invention, the protein scaffold, the SDRs and the engineered enzyme are encoded by a DNA sequence and an expression system is used in order to produce the protein. In an alternative variant, the protein scaffold, the SDRs and/or the engineered enzyme are chemically synthesized from peptide building blocks.

In a fourth variant of this aspect of the invention, the inventive method comprises at least the following steps:

(a) providing a polynucleotide encoding a protein scaffold capable of catalyzing one or more chemical reactions on one or more target substrates;

(b) combining one or more fully or partially random oligonucleotide sequence with the polynucleotide encoding the protein scaffold, the fully or partially random oligonucleotide sequences being located at sites in the polynucleotide that enable the encoded engineered enzyme to discriminate between the one or more target substrates and one or more other substrates; and

(c) selecting out of the population generated in step (b) one or more polynucleotides that encode enzymes that have the defined specificities toward the one or more target substrates.

Any enzyme can serve as the protein scaffold in step (a). It can be a naturally occurring enzyme, a variant or a truncated derivate therefore, or an engineered enzyme. For human therapeutic use, the protein scaffold is preferably a mammalian enzyme, and more preferably a human enzyme. In that aspect, the invention is directed to a method for the generation of essentially mammalian, especially of essentially human enzymes with specificities that are different from specificities of any enzyme encoded in mammalian genomes or in the human genome, respectively.

According to the invention, the protein scaffold provided in step (a) of this aspect requires to be capable of catalyzing one or more chemical reactions on a target substrate. Therefore, a protein scaffold is selected from the group of potential protein scaffolds by its activity on the target substrate.

In a preferred variant of this aspect of the invention, a protein scaffold with hydrolase activity is used. Preferably, a protein scaffold with proteolytic activity is used, and more preferably, a protease with very low specificity having basic activity on the target substrate is used as the protein scaffold. Examples of proteases from different structural classes with low substrate specificity are Papain, Trypsin, Chymotrypsin, Subtilisin, SET (trypsin-like serine protease from Streptomyces erythraeus), Elastase, Cathepsin G or Chymase. Before being employed as the protein scaffold, the amino acid sequence of the protease may be modified in order to change protein properties other than specificity, e.g catalytic activity, stability, inhibitor sensitivity, or expression yield, essentially as described in WO 92/18645, or in order to change specificity, essentially as described in EP 02020576.3 and PCT/EP03/04864.

Another option for a feasible protein scaffold are lipases. Hepatic lipase, lipoprotein lipase and pancreatic lipase belong to the “lipoprotein lipase superfamily”, which in turn is an example of the GX-class of lipases (M. Fischer, J. Pleiss (2003), Nucl. Acid. Res., 31, 319-321). The substrate specificity of lipases can be characterized by their relative activity towards triglycerol esters of fatty acids and phospholipids, bearing a charged head group. Alternatively, other hydrolases such as esterases, glycosylases, amidases, or nitrilases may be used as scaffolds.

Transferases are also feasible protein scaffolds. Glycoslytransferases are involved in many biological synthesis involving a variety of donors and acceptors. Alternatively, the protein scaffold may have ligase, lyase, oxidoreductase, or isomerase activity.

In a first embodiment, the one or more fully or partially random peptide sequences are inserted at specific sites in the protein scaffold. These insertion sites are characterized by the fact that the inserted peptide sequences can act as discriminators between different substrates, i.e. as Specificity Determining Regions or SDRs. Such insertion sites can be identified by several approaches. Preferably, insertion sites are identified by analysis of the three-dimensional structure of the protein scaffolds, by comparative analysis of the primary sequences of the protein scaffold with other enzymes having different quantitative specificities, or experimentally by techniques such as alanine scanning, random mutagenesis, or random deletion, or by any combination thereof.

A first approach to identify insertion sites for SDRs bases on the three-dimensional structure of the protein scaffold as it can be obtained by x-ray crystallography or by nuclear magnetic resonance studies. Structural alignment of the protein scaffold in comparison with other enzymes of the same structural class but having different quantitative specificities reveals regions of high structural similarity and regions with low structural similarity. Such an analysis can for example be done using public software such as Swiss PDB viewer (Guex, N. and Peitsch, M. C. (1997) Electrophoresis 18, 2714-2723). Regions of low structural similarity are preferred SDR insertion sites.

In a second approach to identify insertion sites for SDRs, three-dimensional structures of the scaffold protein in complex with competitive inhibitors or substrate analogs are analysed. It is assumed that the binding site of a competitive inhibitor significantly overlaps with the binding site of the substrate. In that case, atoms of the protein that are within a certain distance of atoms of the inhibitor are likely to be in a similar distance to the substrate as well. Choosing a short distance, e.g. <5 Å, will result in an ensemble of protein atoms that are in close contact with the substrate. These residues would constitute the first shell contacts and are therefore preferred insertion sites for SDRs. Once first shell contacts have been identified, second shell contacts can be found by repeating the distance analysis starting from first shell atoms. In yet another alternative of the invention the distance analysis described above is performed starting from the active site residues.

In third approach to identify insertion sites for SDRs, the primary sequence of the scaffold protein is aligned with other enzymes of the same structural class but having different quantitative specificities using an alignment algorithm. Examples of such alignment algorithms are published (Altschul, S. F., Gish, W., Miller, W., Myers, E. W. & Lipman, D. J. (1990) J. Mol. Biol. 215:403-410; “Statistical methods in Bioinformatics: an introduction” by Ewens, W. & Grant, G. R. 2001, Springer, New York). Such an alignment may reveal conserved and non-conserved regions with varying sequence homology, and, in particular, additional sequence elements in one or more enzymes compared to the scaffold protein. Conserved regions of are more likely to contribute to phenotypes shared among the different proteins, e.g. stabilizing the three-dimensional fold. Non-conserved regions and, in particular, additional sequences in enzymes with quantitatively higher specificity (Turner, R. et al. (2002) J. Biol. Chem., 277, 33068-33074) are preferred insertion sites for SDRs.

For proteases currently five families are known, namely aspartic-, cysteine-, serine-, metallo- and threonine proteases. Each family includes groups of proteases that share a similar fold. Crystallographic structures of members of these groups have been solved and are accessible through public databases, e.g. the Brookhaven protein database (H. M. Berman et al. Nucleic Acids Research, 28 pp. 235-242 (2000)). Such databases also include structural homologs in other enzyme classes and nonenzymatically active proteins of each class. Several tools are available to search public databases for structural homologues: SCOP—a structural classification of proteins database for the investigation of sequences and structures. (Murzin A. G. et al. (1995) J. Mol. Biol. 247, 536-540); CATH—Class, Architecture, Topology and Homologous superfamily: a hierarchical classification of protein domain structures (Orengo et al. (1997) Structure 5(8) 1093-1108); FSSP—Fold classification based on structure-structure alignment of proteins (Holm and Sander (1998) Nucl. Acids Res. 26 316-319); or VAST—Vector alignment search tool (Gibrat, Madej and Bryant (1996) Current Opinion in Structural Biology 6, 377-385).

In the above described approaches, members of structural classes are compared in order to identify insertion sites for SDRs.

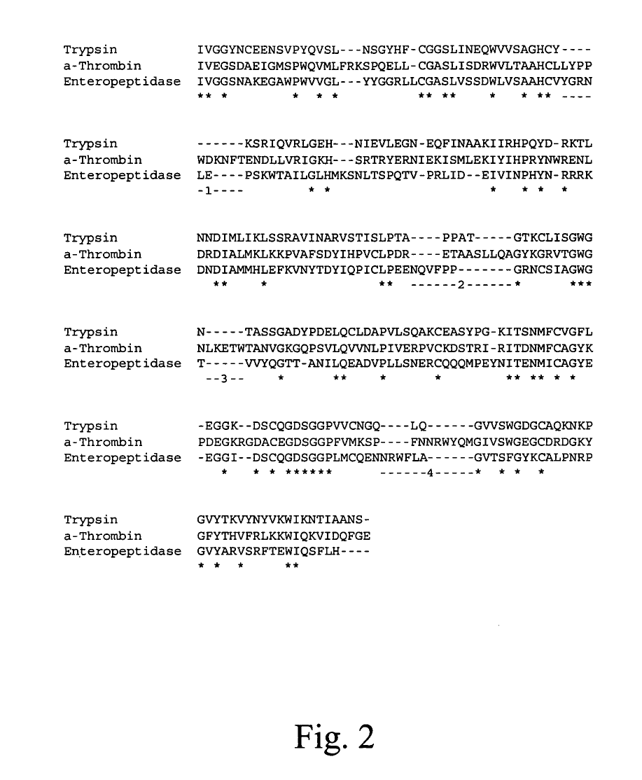

In a preferred variant of these approaches serine proteases of the structural class S1 are compared with each other. Trypsin represents a member with low substrate specificity, as it requires only an arginine or lysine residue at the P₁ position. On the other hand, thrombin, tissue-type plasminogen activator or enterokinase all have a high specificity towards their substrate sequences, i.e. (L/I/V/F)XPR\hat{ }NA, CPGR\hat{ }VVGG and DDDK\hat{ }, respectively (Perona, J. & Craik, C. (1997) J Biol. Chem., 272, 29987-29990; Perona, J. & Craik, C (1995) Protein Science, 4, 337-360). An alignment of the amino acid sequences of these proteases is described in example 1 (FIG. 2) along with the identification of SDRs.

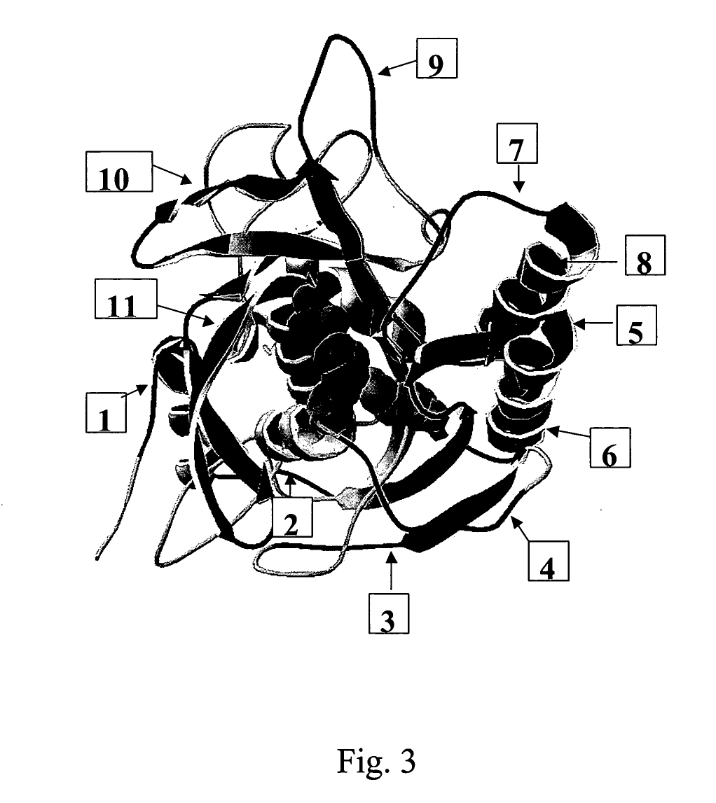

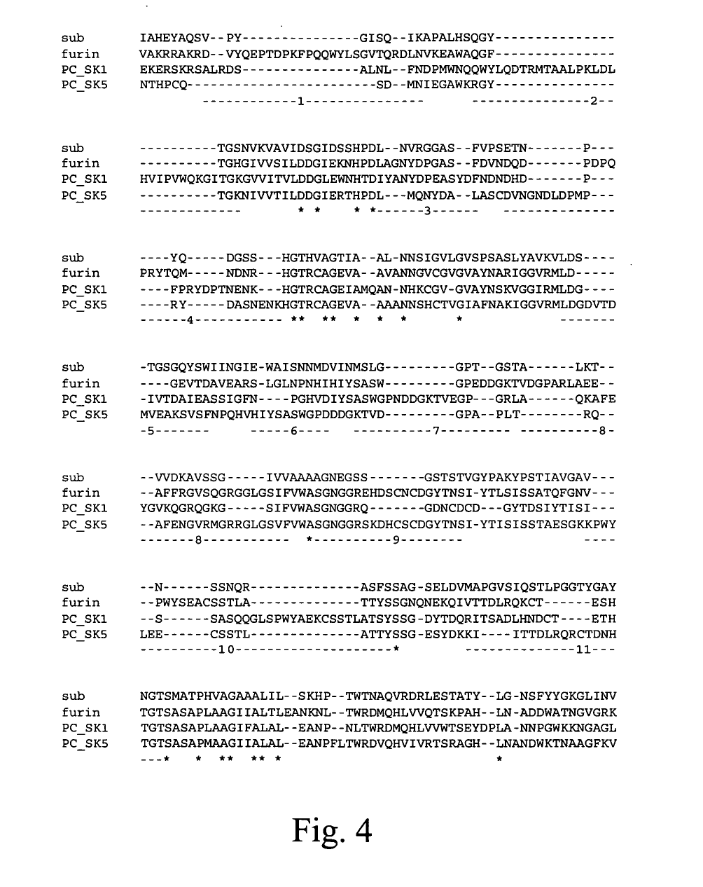

A further example within the family of serine proteases is given by members of the structural class S8 (subtilisin fold). Subtilisin is the type protease for this class and represents an unspecific protease (Ottesen, M. & Svendsen, A. (1998) Methods Enzymol. 19, 199-215). Furin, PC 1 and PC5 are proteases of the same structural class involved in the processing of propeptides and have a high substrate specificity (Seidah, N. & Chretien, M. (1997) Curr. Opin. Biotech., 8: 602-607; Bergeron, F. et al. (2000) J. Mol. Endocrin., 24:1-22). In a preferred variant of the approach alignments of the primary amino acids sequences (FIG. 4) are used to identify eleven sequence stretches longer than three amino acids which specific proteases have in addition compared to subtilisin and are therefore potential specificity determining regions. In a further variant of the approach information from the three-dimensional structure of subtilisin can be used in order to further narrow down the selection (FIG. 3). Out of the eleven inserted sequence stretches, three are especially close to the active site residues, namely stretch number 7, 8 and 11 which are insertions in PC5, PC1 and all three specific proteases, respectively (FIG. 3). In a preferred variant, one or several amino acid stretches of variable length and composition can be inserted into the subtilisin sequence at one or several of the eleven positions. In a more preferred variant of the approach the insertion is performed at regions 7, 8 or 11 or any combination thereof. In another preferred variant of the approach protease scaffolds other than subtilisin from the structural class S8 are used.

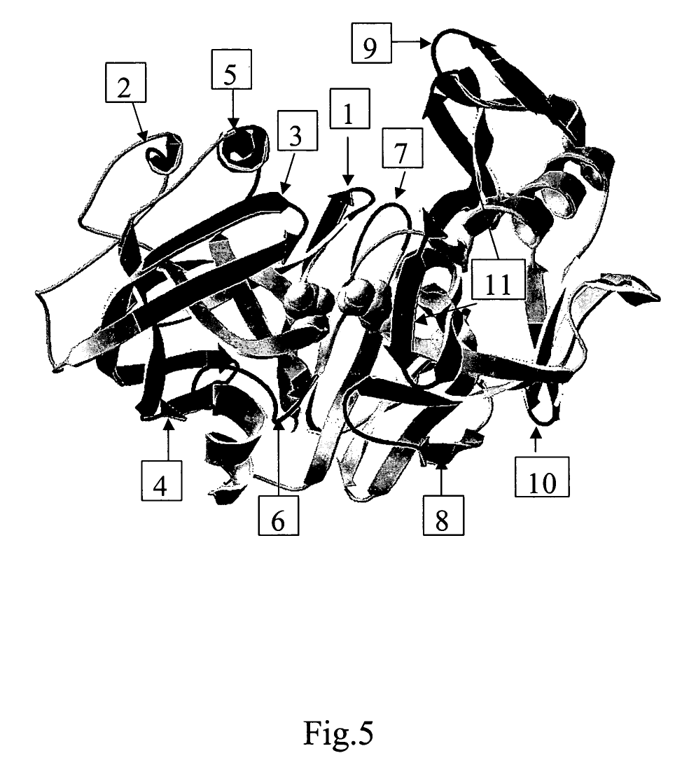

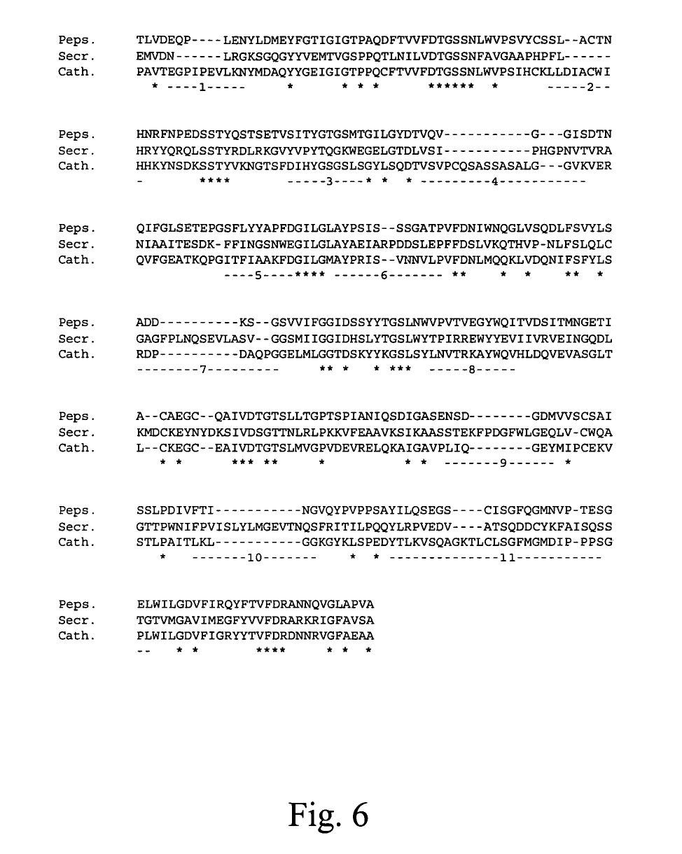

In a further preferred variant of this approach, aspartic acid proteases of the structural class A1 are analyzed (Rawlings, N. D. & Barrett, A. J. (1995). Methods Enzymol. 248, 105-120; Chitpinityol, S. & Crabbe, M J. (1998), Food Chemistry, 61, 395-418). Examples for the Al structural class of aspartic proteases are pepsin with a low as well as beta-secretase (Grüninger-Leitch, F., et al. (2002) J. Biol. Chem. 277, 4687-4693) and renin (Wang, W. & Liang, T C. (1994) Biochemistry, 33, 14636-14641) with relatively high substrate specificities. Retroviral proteases also belong to this class, although the active enzyme is a dimer of two identical subunits. The viral proteases are essential for the correct processing of the polyprotein precursor to generate functional proteins which requires a high substrate specificity in each case (Wu, J. et al. (1998) Biochemistry, 37, 4518-4526; Pettit, S. et al. (1991) J. Biol. Chem., 266, 14539-14547). Pepsin is the type protease for this class and represents an unspecific protease (Kageyama, T. (2002) Cell. Mol. Life Sci. 59, 288-306). B-secretase and Cathepsin D (Aguilar, C. F. et al. (1995) Adv. Exp. Med. Biol. 362, 155-166) are proteases of the same structural class and have a high substrate specificity. In a preferred variant of the approach alignments of the primary amino acids sequences (FIG. 6) are used to identify six sequence stretches longer than three amino acids which are inserted in the specific proteases compared to pepsin and are therefore potential specificity determining regions. In a further variant of the approach information from the three-dimensional structure of b-secretase can be used in order to further narrow down the selection. Out of the six inserted sequence stretches, three are especially close to the active site residues, namely stretch number 1, 3 and 4 which are insertions in cathepsin D and beta-secretase, respectively (FIG. 5). In a preferred variant of the approach, one or several amino acid stretches of variable length and composition can be inserted into the pepsin sequence at one or several of the six positions. In a more preferred embodiment of the invention the insertion is performed at the positions 1, 3 or 4 or any combination thereof. In another preferred embodiment of the invention protease scaffolds other than pepsin are used.

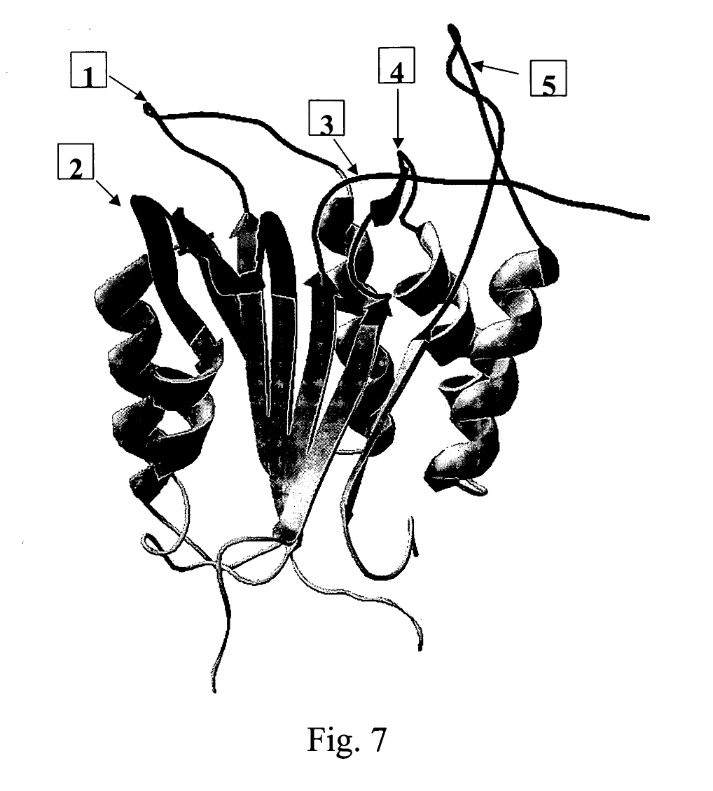

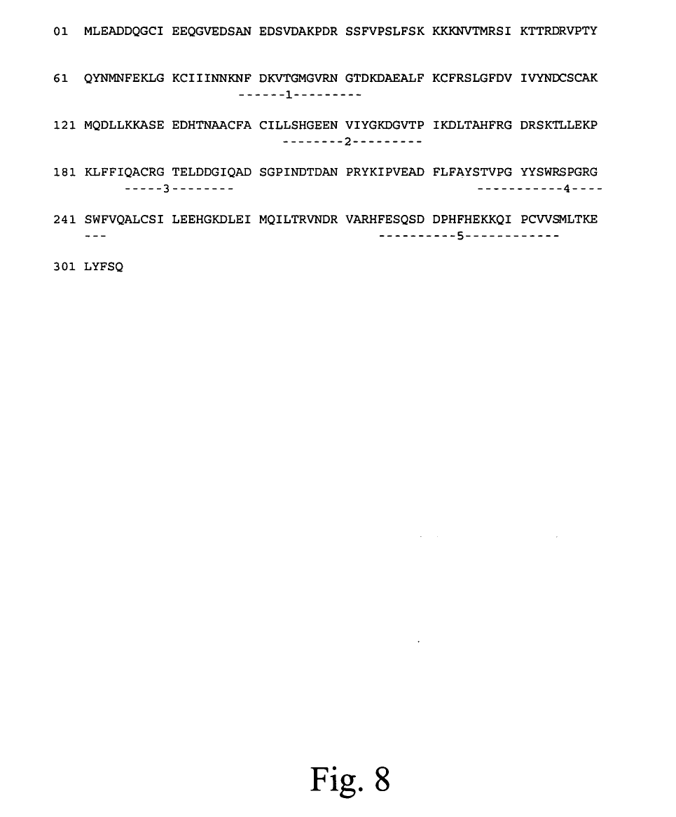

There are cases where a certain structural class does not include known members of low and high specificity. This is exemplified by the C14 class of caspases which belong to the cysteine protease family (Rawlings, N. D. & Barrett, A. J. (1994) Methods Enzymol. 244, 461-486 ) and which all show high specificity for P₄ to P₁ positions. For example, caspase-1, caspase-3 and caspase-9 recognize the sequences YVAD\hat{ }, DEVD\hat{ } or LEHD\hat{ }, respectively. Identification of the regions that differ between the caspases will include the regions responsible for the differences in substrate specificity (FIGS. 7 and 8).

Finally, non-enzymatic proteins of the same fold as the enzyme scaffold may also contribute to the identification of insertion sites for SDRs. For example, haptoglobin (Arcoleo, J. & Greer, J.; (1982) J. Biol. Chem. 257, 10063-10068) and azurocidin (Almeida, R. et al. (1991) Biochem. Biophys. Res. Commun. 177, 688-695) share the same chymotrypsin-like fold with all S1 proteases. Due to substitutions in the active site residues these proteins do not posses any proteolytic function, yet they show high homology with active proteases. Differences between these proteins and specific proteases include regions that can serve as insertion sites for SDRs. In a fourth approach, insertion sites for SDRs are identified experimentally by techniques such as alanine scanning, random mutagenesis, random insertion or random deletion. In contrast to the approach disclosed above, this approach does not require detailed knowledge about the three-dimensional structure of the scaffold protein. In one preferred variant of this approach, random mutagenesis of enzymes with relatively high specificity from the same structural class as the protein scaffold and screening for loss or change of specificity can be used to identify insertion sites for SDRs in the protein scaffold.

Random mutagenesis, alanine scanning, random insertion or random deletion are all done on the level of the polynucleotides encoding the enzymes. There are a variety of protocols known in the literature (e.g. Sambrook, J. F; Fritsch, E. F.; Maniatis, T.; Cold Spring Harbor Laboratory Press, Second Edition, 1989, New York). For example, random mutagenesis can be achieved by the use of a polymerase as described in patent WO 9218645. According to this patent, the one or more genes encoding the one or more proteases are amplified by use of a DNA polymerase with a high error rate or under conditions that increase the rate of misincorporations. For example the method of Cadwell and Joyce can be employed (Cadwell, R. C. and Joyce, G. F., PCR methods. Appl. 2 (1992) 28-33). Other methods of random mutagenesis such as, but not limited to, the use of mutator stains, chemical mutagens or UV-radiation can be employed as well.

Alternatively, oligonucleotides can be used for mutagenesis that substitute randomly distributed amino acid residues with an alanine. This method is generally referred to as alanine scanning mutagenesis (Fersht, A. R. Biochemistry (1989) 8031-8036). As a further alternative, modifications of the alanine scanning mutagenesis such as binominal mutagenesis (Gregoret, L. M. and Sauer, R. T. PNAS (1993) 4246-4250) or combinatorial alanine scanning (Weiss et al., PNAS (2000) 8950-8954) can be employed.

In order to express engineered enzymes, the DNA encoding such engineered proteins is ligated into a suitable expression vector by standard molecular cloning techniques (e.g. Sambrook, J. F; Fritsch, E. F.; Maniatis, T.; Cold Spring Harbor Laboratory Press, Second Edition, 1989, New York). The vector is introduced in a suitable expression host cell, which expresses the corresponding engineered enzyme variant. Particularly suitable expression hosts are bacterial expression hosts such as Escherichia coli or Bacillus subtilis, or yeast expression hosts such as Saccharomyces cerevisae or Pichia pastoris, or mammalian expression hosts such as Chinese Hamster Ovary (CHO) or Baby Hamster Kidney (BHK) cell lines, or viral expression systems such as bacteriophages like M13 or Lambda, or viruses such as the Baculovirus expression system. As a further alternative, systems for in vitro protein expression can be used. Typically, the DNA is ligated into an expression vector behind a suitable signal sequence that leads to secretion of the enzyme variants into the extracellular space, thereby allowing direct detection of protease activity in the cell supernatant. Particularly suitable signal sequences for Escherichia coli are H1yA, for Bacillus subtilis AprE, NprB, Mpr, AmyA, AmyE, B1ac, SacB, and for S. cerevisiae Bar1, Suc2, Matα, Inu1A, Ggp1p. Alternatively, the enzyme variants are expressed intracellularly and the substrates are expressed also intracellularly. Preferably, this is done essentially as described in patent application WO 0212543, using a fusion peptide substrate comprising two auto-fluorescent proteins linked by the substrate amino-acid sequence. As a further alternative, after intracellular expression of the enzyme variants, or secretion into the periplasmatic space using signal sequences such as DsbA, PhoA, Pe1B, OmpA, OmpT or gIII for Escherichia coli, a permeabilisation or lysis step releases the enzyme variants into the supernatant. The destruction of the membrane barrier can be forced by the use of mechanical means such as ultrasonic, French press, or the use of membrane-digesting enzymes such as lysozyme. As another, further alternative, the genes encoding the enzyme variants are expressed cell-free by the use of a suitable cell-free expression system. For example, the S30 extract from Escherichia coli cells is used for this purpose as described by Lesly et al. (Methods in Molecular Biology 37 (1995) 265-278).

The ensemble of gene variants generated and expressed by any of the above methods are analyzed with respect to their affinity, substrate specificity or activity by appropriate assay and screening methods as described in detail for example in patent application PCT/EP03/04864. Genes from catalytically active variants having reduced specificity in comparison to the original enzyme are analyzed by sequencing. Sites at which mutations and/or insertions and/or deletions occurred are preferred insertion sites at which SDRs can be inserted site-specifically.

In a second embodiment, the one or more fully or partially random peptide sequences are inserted at random sites in the protein scaffold. This modification is usually done on the polynucleotide level, i.e. by inserting nucleotide sequences into the gene that encodes the protein scaffold. Several methods are available that enable the random insertion of nucleotide sequences. Systems that can be used for random insertion are for example ligation based systems (Murakami et al. Nature Biotechnology 20 (2002) 76-81), systems based on DNA polymerisation and transposon based systems (e.g. GPS-M™ mutagenesis system, NEB Biolabs; MGS™ mutation generation system, Finnzymes). The transposon-based methods employ a transposase-mediated insertion of a selectable marker gene that contains at its termini recognition sequences for the transposase as well as two sites for a rare cutting restriction endonuclease. Using the latter endonuclease one usually releases the selection marker and after religation obtains an insertion. Instead of performing the religation one can alternatively insert a fragment that has terminal recognition sequences for one or two outside cutting restriction endonuclease as well as a selectable marker. After ligation, one releases this fragment using the one or two outside cutting endonucleases. After creating blunt ends by standard methods one inserts blunt ended random fragments at random positions into the gene.

In a further preferred embodiment, methods for homologous in-vitro recombination are used to combine the mutations introduced by the above mentioned methods to generate enzyme populations. Examples of methods that can be applied are the Recombination Chain Reaction (RCR) according to patent application WO 0134835, the DNA-Shuffling method according to the patent application WO 9522625, the Staggered Extension method according to patent WO 9842728, or the Random Priming recombination according to patent application WO9842728. Furthermore, also methods for non-homologous recombination such as the Itchy method can be applied (Ostermeier, M. et al. Nature Biotechnology 17 (1999) 1205-1209).

Upon random insertion of a nucleotide sequence into the protein scaffold one obtains a library of different genes encoding enzyme variants. The polynucleotide library is subsequently transferred to an appropriate expression vector. Upon expression in a suitable host or by use of an in vitro expression system, a library of enzymes containing randomly inserted stretches of amino acids is obtained.

According to step (b) of this third aspect of the invention, one or more fully or partially random peptide sequences are inserted into the protein scaffold. The actual number of such inserted SDRs is determined by the intended quantitative specificity following the relation: the higher the intended specificity is, the more SDRs are inserted. Whereas a single SDR enables the generation of moderately specific enzymes, two SDRs enable already the generation of significantly specific enzymes. However, up to six and more SDRs can be inserted into a protein scaffold. A similar relation is valid for the length of the SDRs: the higher the intended specificity is, the longer are the SDRs that are to be inserted. SDRs can be as short as one to four amino acid residues. They can, however, also be as long as 50 amino acid residues. Significant specificity can already be generated by the use of SDRs of a length of four to six amino acid residues.

The peptid sequences that are inserted can be fully or partially random. In this context, fully random means that a set of sequences are inserted in parallel that includes sequences that differ from each other in each and every position. Partially random means that a set of sequences are inserted in parallel that includes sequences that differ from each other in at least one position. This difference can be either pair-wise or with respect to a single sequence. For example, when regarding an insertion of the length of four amino acids, partial random could be a set (i) that includes AGGG, GVGG, GGLG, GGGI, or (ii) that includes AGGG, VGGG, LGGG and IGGG. Alternatively, random sequences also comprises sequences that differ from each other in length. Randomization of the peptide sequences is achieved by randomization of the nucleotide sequences that are inserted into the gene at the respective sites. Thereby, randomization can be achieved by employing mixtures of nucleobases as monomers during chemical synthesis of the oligonucleotides. A particularly preferred mixture of monomers for a fully random codon that in addition minimizes the probability of stop codons is NN(GTC). Alternatively, random oligonucleotides can be obtained by fragmentation of DNA into short fragments that are inserted into the gene at the respective sites. The source of the DNA to be fragmented may be a synthetic oligonucleotide but alternatively may originate from cloned genes, cDNAs, or genomic DNA. Preferably, the DNA is a gene encoding an enzyme. The fragmentation can, for example, be achieved by random endonucleolytic digestion of DNA. Preferably, an unspecific endonuclease such as DNAse I (e.g. from bovine pancreas) is employed for the endonucleolytic digestion.

If steps (a)-(c) of the inventive method are repeated cyclically, there are different alternatives for obtaining random peptide sequences that are inserted in consecutive rounds. Preferably, SDRs that were identified in one round as leading to increased specificity of enzyme are used as templates for the random peptide sequences that are inserted in the following round.

In a preferred alternative, the sequences selected in one round are analysed and randomized oligonucleotides are generated based on these sequences. This can, for example, be achieved by using in addition to the original nucleotide with a certain percentage mixtures of the other three nucleotides monomers at each position in the oligonucleotide synthesis. If, for example, in a first round an SDRs is identified that has the amino acid sequence ARLT, e.g. encoded by the nucleotide sequence GCG CGC CTT ACC, a random peptide sequence inserted in this SDR site could be encoded by an oligonucleotide with 70% G, 10% A, 10% T and 10% C at the first position, 70% C, 10% G, 10% T and 10% A at the second position, etc. This leads at each position approximately in 1 of 3 cases to the template amino acid and in 2 of 3 cases to another amino acid.

In another preferred alternative, the sequences selected in one round are analyzed and a consensus library is generated based on these sequences. This can, for example, be achieved by using defined mixtures of nucleotides at each position in the oligonucleotide synthesis in a way that leads to mixtures of the amino acid residues that were identified at each position of the SDR selected in the previous round. If, for example, in a first round two SDRs are identified that have the amino acid sequences ARLT and VPGS, a consensus library inserted in this SDR site in the following round could be encoded by an oligonucleotide with the sequence G(C/T)G C(G/C)C (G/T)(G/T)G (A/T)CC. This would correspond to the random peptide sequence (A/V)(R/P)(L/G/V/W)(TIS), thereby allowing all combinations of the amino acid residues identified in the first round, and, due to the degeneracy of the genetic code, allowing in addition to a lower degree alternative amino acid residues at some positions.

In another preferred alternative, the sequences selected in one round are, without previous analysis, recombined using methods for the in vitro recombination of polynucleotides, such as the methods described in WO 01/34835 (the following also provides details of the eighth and ninth aspect of the invention).

After insertion of the partially or fully random sequences into the gene encoding the scaffold protein, and eventually ligation of the resulting gene into a suitable expression vector using standard molecular cloning techniques (Sambrook, J. F; Fritsch, E. F.; Maniatis, T.; Cold Spring Harbor Laboratory Press, Second Edition, 1989, New York), the vector is introduced in a suitable expression host cell which expresses the corresponding enzyme variant. Particularly suitable expression hosts are bacterial expression hosts such as Escherichia coli or Bacillus subtilis, or yeast expression hosts such as Saccharomyces cerevisae or Pichia pastoris, or mammalian expression hosts such as Chinese Hamster Ovary (CHO) or Baby Hamster Kidney (BHK) cell lines, or viral expression systems such as bacteriophages like M13 T7 phage or Lambda, or viruses such as the Baculovirus expression system. As a further alternative, systems for in vitro protein expression can be used. Typically, the DNA is ligated into an expression vector behind a suitable signal sequence that leads to secretion of the enzyme variants into the extracellular space, thereby allowing direct detection of enzyme activity in the cell supernatant. Particularly suitable signal sequences for Escherichia coli are ompA, pe1B, H1yA, for Bacillus subtilis AprE, NprB, Mpr, AmyA, AmyE, B1ac, SacB, and for S. cerevisiae Bar1, Suc2, Matα, Inu1A, Ggp1p. Alternatively, the enzyme variants are expressed intracellularly and the substrates are expressed also intracellularly. According to protease variants this is done essentially as described in patent application WO 0212543, using a fusion peptide substrate comprising two auto-fluorescent proteins linked by the substrate amino-acid sequence. As a further alternative, after intracellular expression of the enzyme variants, or secretion into the periplasmatic space using signal sequences such as DsbA, PhoA, Pe1B, OmpA, OmpT or gIII for Escherichia coli, a permeabilisation or lysis step releases the enzyme variants into the supernatant. The destruction of the membrane barrier can be forced by the use of mechanical means such as ultrasonic, French press, or the use of membrane-digesting enzymes such as lysozyme. As another, further alternative, the genes encoding the enzyme variants are expressed cell-free by the use of a suitable cell-free expression system. For example, the S30 extract from Escherichia coli cells is used for this purpose as described by Lesly et al. (Methods in Molecular Biology 37 (1995) 265-278).

After introduction of the vector into host cells, these cells are screened for the expression of enzymes with specificity for the intended target substrate. Such screening is typically done by separating the cells from each other, in order to enable the correlation of genotype and phenotype, and assaying the activity of each cell clone after a growth and expression period. Such separation can for example be done by distribution of the cells into the compartments of sample carriers, e.g. as described in WO 01/24933. Alternatively, the cells are separated by streaking on agar plates, by enclosing in a polymer such as agarose, by filling into capillaries, or by similar methods.

Identification of variants with the intended specificity can be done by different approaches. In the case of proteases, preferably assays using peptide substrates essentially as described in PCT/EP03/04864 are employed.

Regardless of the expression format, selection of enzyme variants is done under conditions that allow identification of enzymes that recognize and convert the target sequence preferably. As a first alternative, enzymes that recognize and convert the target sequence preferably are identified by screening for enzymes with a high affinity for the target substrate sequence. High affinity corresponds to a low K_{M} which is selected by screening at target substrate concentrations substantially below the K_{M} of the first enzyme. Preferably, the substrates that are used are linked to one or more fluorophores that enable the detection of the modification of the substrate at concentrations below 10 μM, preferably below 1 μM, more preferably below 100 nM, and most preferably below 10 nM.

As a second alternative, enzymes that recognize and convert the target substrate preferably are identified by employing two or more substrates in the assay and screening for activity on these two or more substrates in comparison. Preferably, the two or more substrates employed are linked to different marker molecules, thereby enabling the detection of the modification of the two or more substrates consecutively or in parallel. In the case of proteases, particularly preferably two peptide substrates are employed, one peptide substrate having an arbitrarily chosen or even partially or fully random amino-acid sequence thereby enabling to monitor the activity on an arbitrary substrate, and the other peptide substrate having an amino-acid sequence identical to or resembling the intended target substrate sequence thereby enabling to monitor the activity on the target substrate. Especially preferably, these two peptide substrates are linked to fluorescent marker molecules, and the fluorescent properties of the two peptide substrates are sufficiently different in order to distinguish both activities when measured consecutively or in parallel. For example, a fusion protein comprising a first autofluorescent protein, a peptide, and a second autofluorescent protein according to patent application WO 0212543 can be used for this purpose. Alternatively, fluorophores such as rhodamines are linked chemically to the peptide substrates.

As a third alternative, enzymes that recognize and convert the target substrate preferably are identified by employing one or more substrates resembling the target substrate together with competing substrates in high excess. Screening with respect to activity on the substrates resembling the target substrate is then done in the presence of the competing substrates. Enzymes having a specificity which corresponds qualitatively to the target specificity, but having only a low quantitative specificity are identified as negative samples in such a screen. Whereas enzymes having a specificity which corresponds qualitatively and quantitatively to the target specificity are identified positively. Preferably, the one or more substrates resembling the target substrate are linked to marker molecules, thereby enabling the detection of their modifications, whereas the competing substrates do not carry marker molecules. The competing substrates have arbitrarily chosen or random amino-acid sequences, thereby acting as competitive inhibitors for the hydrolysis of the marker-carrying substrates. For example, protein hydrolysates such as Trypton can serve as competing substrates for engineered proteolytic enzymes according to the invention.

As a fourth alternative, enzymes that recognize and convert the target substrate preferably are identified and selected by an amplification-coupled or growth-coupled selection step. Furthermore, the activity can be measured intracellularily and the selection can be done by a cell sorter, such as a fluorescence-activated cell sorter.

As a further alternative, enzymes that recognize and convert the target substrate are identified by first selecting enzymes that preferentially bind to the target substrate, and secondly selecting out of this subgroup of enzyme variants those enzymes that convert the target substrate. Selection for enzymes that preferentially bind the target substrate can be either done by selection of binders to the target substrate or by counter-selection of enzymes that bind to other substrates. Methods for the selection of binders or for the counter-selection of non-binders is known in the art. Such methods typically require phenotype-genotype coupling which can be solved by using surface display expression methods. Such methods include, for example, phage or viral display, cell surface display and in vitro display. Phage or viral display typically involves fusion of the protein of interest to a viral/phage protein. Cell surface display, i.e. either bacterial or eukaryotic cell display, typically involves fusion of the protein of interest to a peptide or protein that is located at the cell surface. In in-vitro display, the protein is typically made in vitro and linked directly or indirectly to the mRNA encoding the protein (DE 19646372).

The invention also provides for a composition or pharmaceutical composition comprising one or more engineered enzymes according to the first aspect of the invention as defined herein before. The composition may optionally comprise an acceptable carrier, excipient and/or auxiliary agent.

Pharmaceutical compositions according to the invention may optionally comprise a pharmaceutically acceptable carrier. Pharmaceutical formulations are well known and pharmaceutical compositions may be routinely formulated by one having ordinary skill in the art. The composition can be formulated as a solution, suspension, emulsion, or lyophilized powderin association with a pharma-ceutically acceptable vehicle. Examples of such vehicles are water, saline, Ringer's solution, dextrose solution, and human serum albumin. Liposomes and nonaaqueous vehicles such as fixed oils may also be used. The vehicle or lyophilized powder may contain additives that maintain isotonicity (e.g. sodium chloride, mannitol) and chemical stability (e.g. buffers and preservatives). The composition is sterilized by commonly used techniques.

The pharmaceutical composition of the present invention may be administrated by any means that enables the active agent to reach the agent's site of action in the body of a mammal. Pharmaceutical compositions may be administered parentally, i.e. intravenous, subcutaneous, intramuscular.

Dosage varies depending upon known factors such as the pharmacodynamic characteristics of the particular agent, and its mode and route of administration; age, health, and weight of the recipient; nature and extent of symptoms, kind of concurrent treatment, frequency of treatment, and the effect desired.

Non-pharamceutical compositions as defined herein are research composition, nutritional composition, cleaning composition, desinfection composition, cosmetic composition or composition for personal care. Moreover, DNA sequences coding for the engineered enzyme as defined herein before and vectors containing said DNA sequences are also provided. Finally, transformed host cells (prokaryotic or eukaryotic) or transgenic organisms containing such DNA sequences and/or vectors, as well as a method utilizing such host cells or transgenic animals for producing the engineered enzyme of the first aspect of the invention are also contemplated.

### DETAILED DESCRIPTION OF THE FIGURES

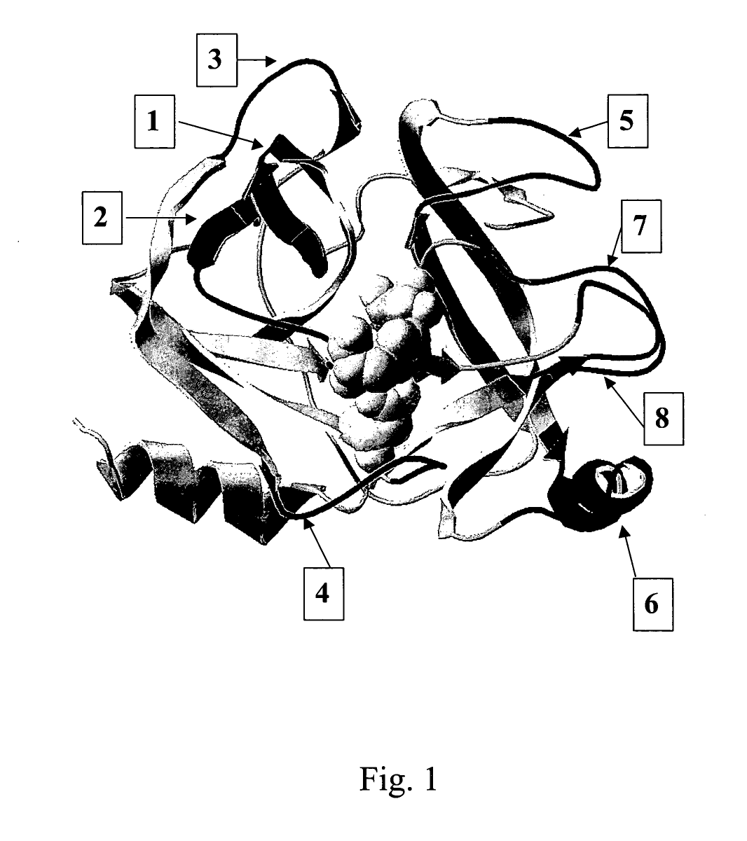

FIG. 1: Three-dimensional structure of human trypsin I with the active site residues shown in “ball-and-stick” representation and with the marked regions indicating potential SDR insertion sites.

FIG. 2: Alignment of the primary amino acid sequences of the human proteases trypsin I, alpha-thrombin and enteropeptidase all of which belong to the structural class S1 of the serine protease family. Trypsin represents an unspecific protease of this structural class, while alpha-thrombin and enteropeptidase are proteases with high substrate specificity. Compared to trypsin several regions of insertions of three or more amino acids into the primary sequence of a-thrombin and enterokinase are seen. The region marked with (-1-) and the region marked with (-3-) are preferred SDR insertion sites. In the tertiary structure of alpha-thrombin both regions are in the vicinity of the substrate binding site. These regions therefore fullfil two criteria to be selected as candidates for SDRs: firstly, they represent insertions in the specific proteases compared to the unspecific one and, secondly, they are close to the substrate binding site. A representation of the three-dimensional structure is given in FIG. 3.

FIG. 3: Three-dimensional structure of subtilisin with the active site residues being shown in “ball-and-stick” representation and with the numbered regions indicating potential SDR insertion sites.

FIG. 4: Alignment of the primary amino acid sequences of subtilisin E, furin, PC 1 and PC5 all of which belong to the structural class S8 of the serine protease family. Subtilisin E represents an unspecific protease of this structural class, while furin, PC 1 and PC5 are proteases with high substrate specificity. Compared to subtilisin several regions of insertions of three or more amino acids into the primary sequence of furin, PC1 and PC5 are seen. The regions marked with (-4-), (-5-), (-7-), (-9-) and (-11-) are preferred SDR insertion sites. These regions stretches fulfill two criteria to be selected as candidates for SDRs: firstly, they represent insertions in the specific proteases compared to the unspecific one and, secondly, they are close to the active site residues.

FIG. 5: Three-dimensional structure of beta-secretase with the active site residues being shown in “ball-and-stick” representation and with the numbered regions indicating potential SDR insertion sites.

FIG. 6: Alignment of the primary amino acid sequences of pepsin, b-secretase and cathepsin D, all of which belong to the structural class Al of the aspartic protease family. Pepsin represents an unspecific protease of this structural class, while b-secretase and cathepsin D are proteases with high substrate specificity. Compared to pepsin several regions of insertions of three or more amino acids into the primary sequence of b-secretase and cathepsin D are seen. The regions marked with -1- to -11- correspond to possible SDR combining sites and are also marked in FIG. 5.

FIG. 7: illustrates the three-dimensional structure of caspase 7 with the active site residues being shown in “ball-and-stick” representation and with the numbered regions indicating potential SDR insertion sites.

FIG. 8: shows the primary amino acid sequence of caspase 7 as a member of the cysteine protease class C14 family (see also SEQ ID NO: 14).

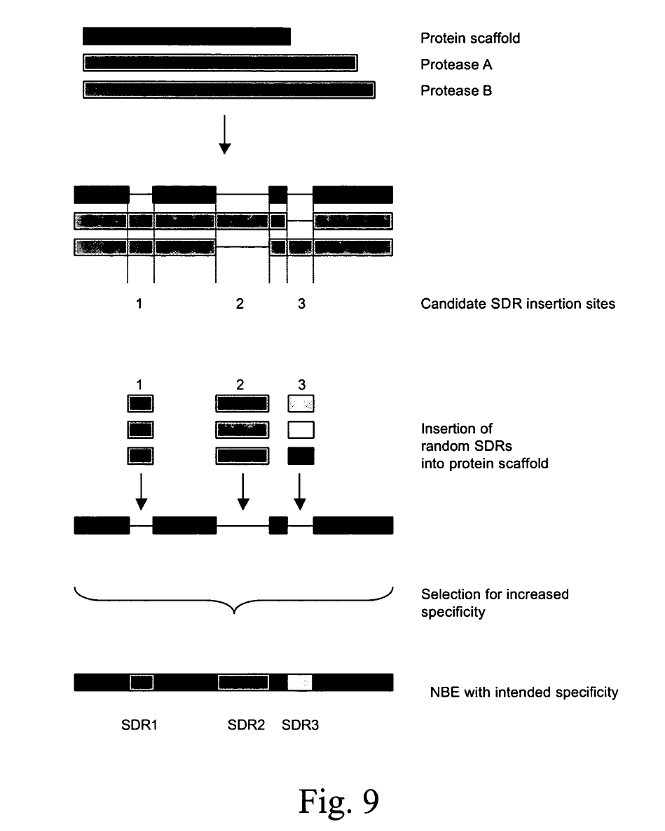

FIG. 9: Schematic representation of method according to the third aspect of the invention.

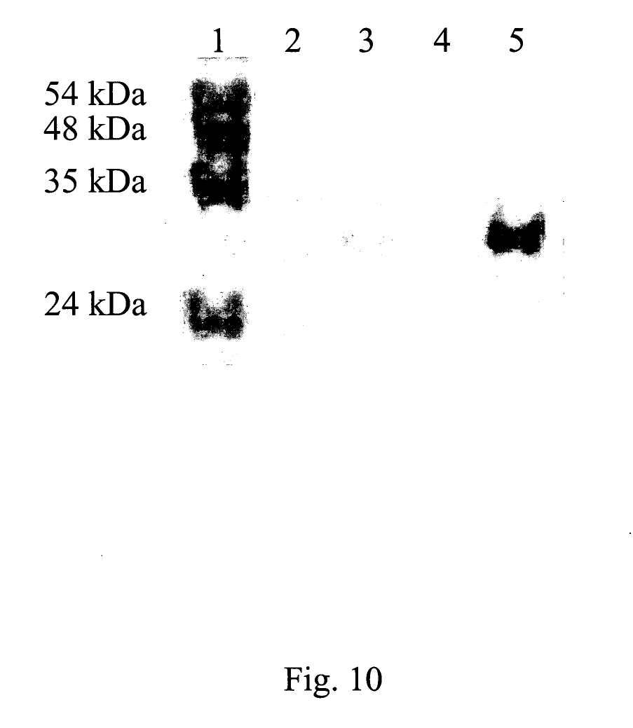

FIG. 10: Western blot analysis of trypsin expression. Supernatant of cell cultures expressing variants of trypsin are compared to negative controls. Lane 1: molecular weight standard; lane 2: negative control; lane 3: supernatant of variant a; lane 4: negative control; lane 5: supernatant of variant b. A primary antibody specific to the expressed protein and a secondary antibody for generation of the signal were used.

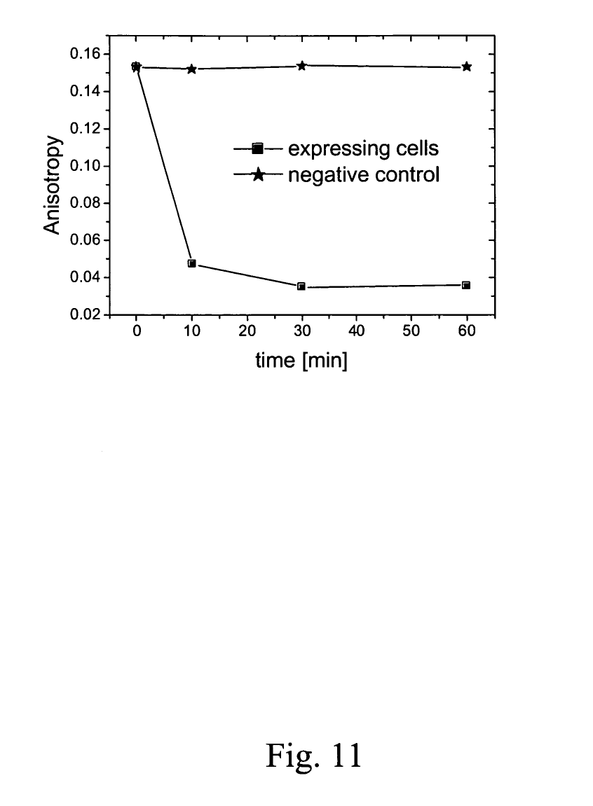

FIG. 11: Time course of the proteolytic cleavage of a target substrate. Supernatant of cells containing the vector with the gene for human trypsin and that of cells containing the vector without the gene was incubated with the peptide substrate described in the text. Cleavage of the peptide results in a decreased read out value. Proteolytic activity is confirmed for the positive clone.

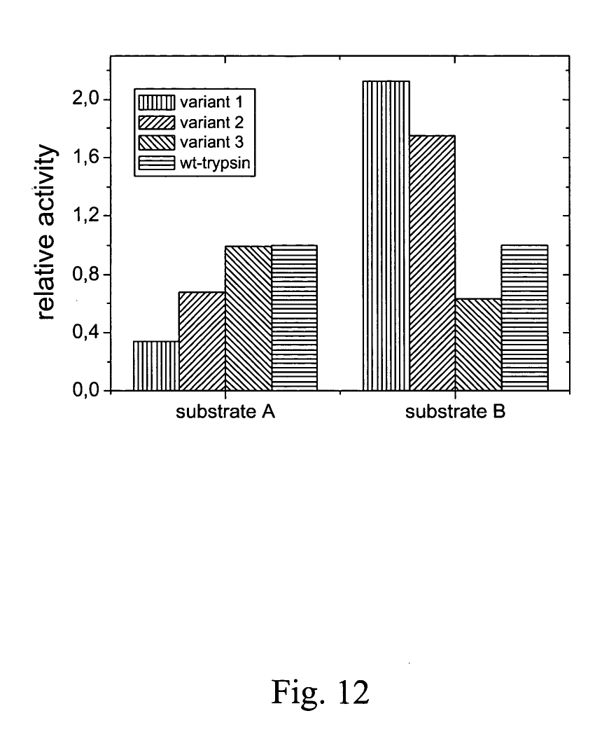

FIG. 12: Relative activity of three engineered proteolytic enzymes in comparison with human trypsin I on two different peptide substrates. A time course of the proteolytic digestion of the two substrates was performed and evaluated. Substrate B was used for screening and substrate A is a closely related sequence. Relative activity of the three variants was normalized to the activity of human trypsin I. Variant 1 and 2 clearly show increased specificity towards the target substrate. Variant 3, on the other hand, serves as a negative control with similar activities as the human trypsin I.

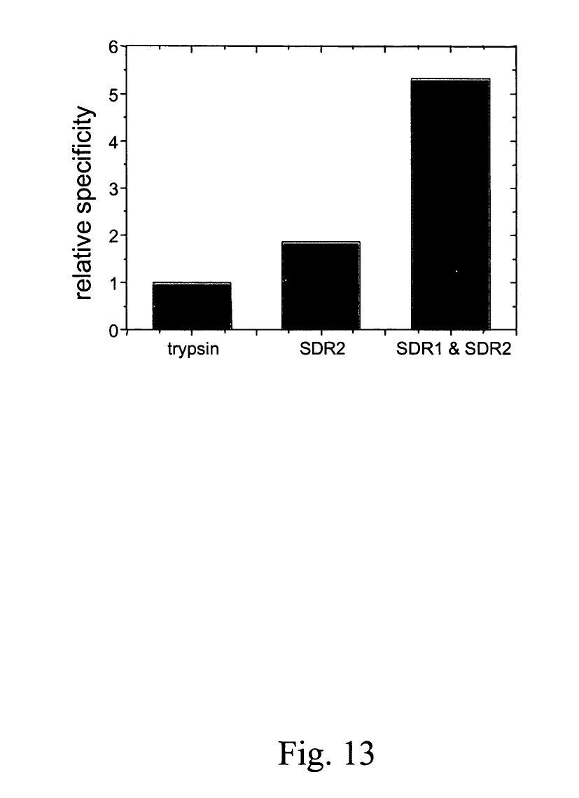

FIG. 13: Relative specificities of trypsin and variants of engineered proteolytic enzymes with one or two SDRs, respectively. Activity of the proteases was determined in the presence and absence of competitor substrate, i.e. peptone at a concentration of 10 mg/ml. Time courses for the proteolytic cleavage were recorded and the time constants k determined. The ratios between the time constants with and without competitor were formed and represent a quantitative measure for the specificity of the protease. The ratios were normalized to trypsin. The specificity of the variant containing two SDRs is 2.5 fold higher than that of the variant with SDR2 alone.

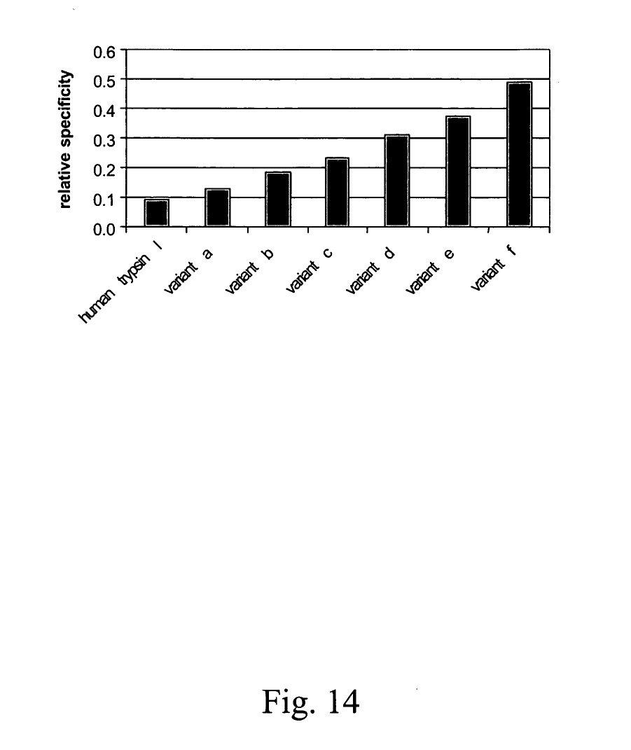

FIG. 14: Shows the relative specificities of protease variants in absence and presence of competitor substrate. The protease variants containig two inserts with different sequences and the non-modified scaffold human trypsin I were expressed in a suitable host. Activity of the protease variants was determined as the cleavage rate of a peptide with the desired target sequence of TNF-alpha in the absence and presence of competitor substrate. Specificity is expressed as the ratio of cleavage rates in the presence and absence of competitor.

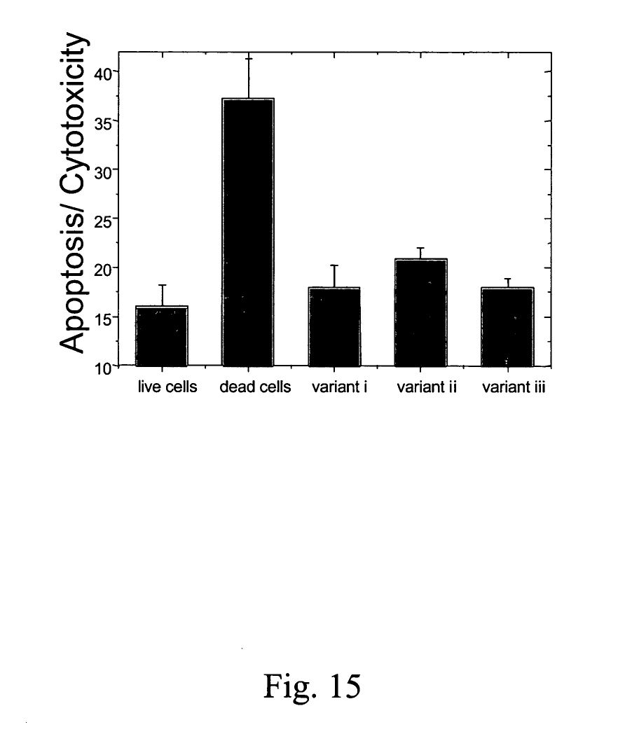

FIG. 15: The figure shows the reduction of cytotoxicity induced by human TNF-alpha when incubating the human TNF-alpha with concentrated supernatant from cultures expressing the inventive engineered proteolytic enzymes being specific for human TNF-alpha. This indicates the efficacy of the inventive engineered proteolytic enzymes.

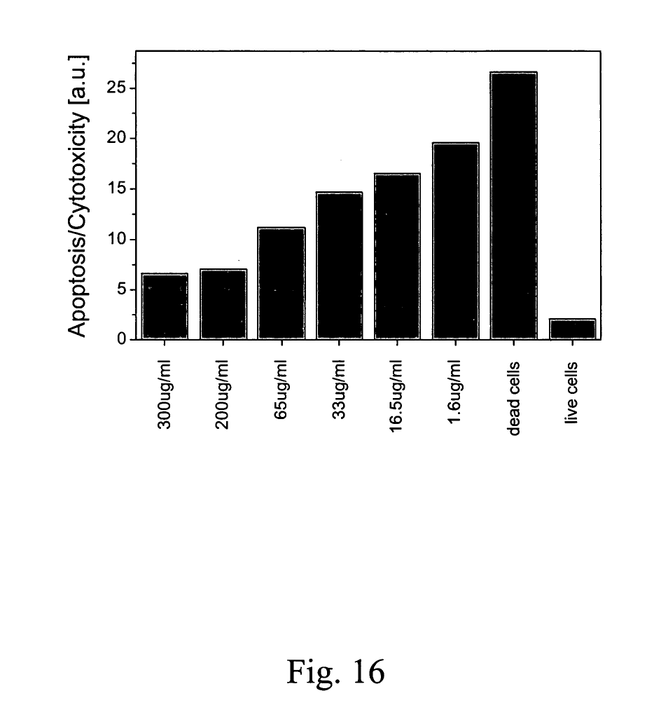

FIG. 16: The figure shows the reduction of cytotoxicity induced by human TNF-alpha when incubating the human TNF-alpha with different concentrations of purified inventive engineered proteolytic enzyme being specific for human TNF-alpha. Variant g comprises SEQ ID NO:72 as SDR1 and SEQ ID NO:73 as SDR2. This indicates the efficacy of the inventive engineered proteolytic enzymes.

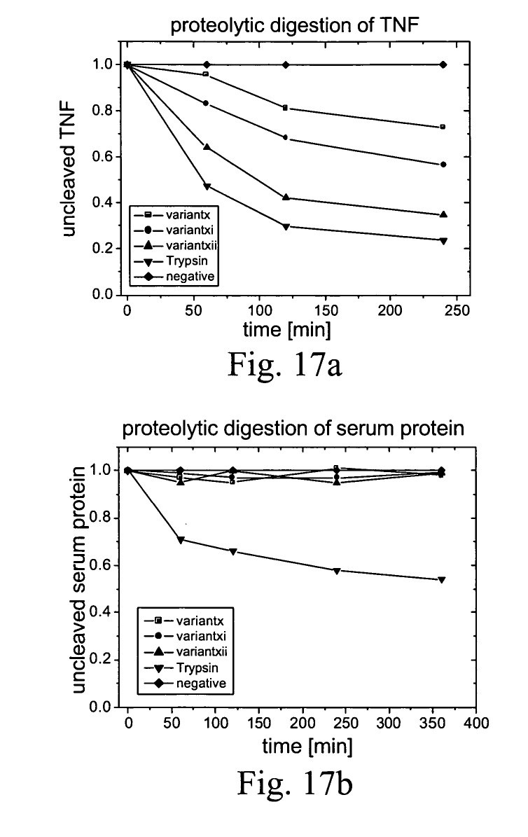

FIG. 17: The figure compares the activity of inventive engineered proteolytic enzymes being specific for human TNF-alpha with the activity of human trypsin I on two protein substrates: (a) human TNF-alpha; (b) mixture of human serum proteins. This indicates the safety of the inventive engineered proteolytic enzymes. Variant x corresponds to Seq ID No: 75 comprising the SDRs according to Seq ID No. 89 (SDR1) and 95 (SDR2). Variants xi and xii correspond to derivatives thereof comprising the same SDR sequences.

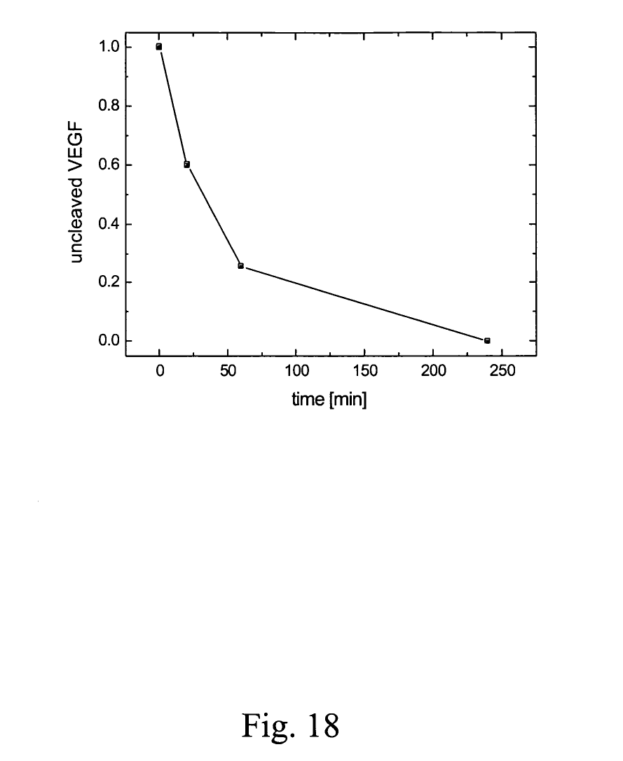

FIG. 18: Specific hydrolysis of human VEGF by an engineered proteolytic enzyme derived from human trypsin.

### EXAMPLES

In the following examples, materials and methods of the present invention are provided including the determination of catalytic properties of enzymes obtained by the method. It should be understood that these examples are for illustrative purpose only and are not to be construed as limiting this invention in any manner. All publications, patents, and patent applications cited herein are hereby incorporated by reference in their entirety for all purposes.

In the experimental examples described below, standard techniques of recombinant DNA technology were used that were described in various publications, e.g. Sambrook et al. (1989), Molecular Cloning: A Laboratory Manual, Cold Spring Harbor Laboratory, or Ausubel et al. (1987), Current Protocols in Molecular Biology 1987-1988, Wiley Interscience. Unless otherwise indicated, restriction enzymes, polymerases and other enzymes as well as DNA purification kits were used according to the manufacturers specifications.

### Example I

Identification of SDR Sites in Human Trypsin

Insertion sites for SDRs have been identified in the serine protease human trypsin I (structural class S1) by comparison with members of the same structural class having a higher sequence specificity. Trypsin represents a member with low substrate specificity, as it requires only an arginine or lysine residue at the P₁ position. On the other hand, thrombin, tissue-type plasminogen activator or enterokinase all have a high specificity towards their substrate sequences, i.e. (L/I/V/F)XPR\hat{ }NA, CPGR\hat{ }VVGG and DDDK\hat{ }, respectively. The primary sequences and tertiary structures of these and further S1 serine proteases have been aligned in order to determine regions of low and high sequence and structure homology and especially regions that correspond to insertions in the sequences of the more specific proteases (FIG. 2). Several regions of insertions equal or longer than 3 amino acids representing potential SDR sites have been identified as indicated in FIG. 1. These regions were chosen as target sites for the insertion of SDRs in the examples below, e.g. SDR1 (region one in FIG. 2, after amino acid 42 according to SEQ ID NO:1) with a length of six and SDR2 (region three in FIG. 2, after amino acid 123 according to SEQ ID NO:1) with a length of five amino acids, respectively.

### Example II

Molecular Cloning of the Human Trypsin I Gene to be Used as Scaffold Protein and Expression of the Mature Protease in B. subtilis

The gene encoding the unspecific protease human trypsinogen I was cloned into the vector pUC18. Cloning was done as follows: the coding sequence of the protein was amplified by PCR using primers that introduced a KpnI site at the 5′ end and a BamHI site at the 3′ end. This PCR fragment was cloned into the appropriate sites of the vector pUC 18. Identity was confirmed by sequencing. After sequencing the coding sequence of the mature protein was amplified by PCR using primers that introduced different Bg1I sites at the 5′ end and the 3′ end.

This PCR fragment was cloned into the appropriate sites of an E. coli-B. subtilis shuttle vector. The vector contains a pMB1 origin for amplification in E. coli, a neomycin resistance marker for selection in E. coli, as well as a P43 promoter for the constitutive expression in B. subtilis. A 87 bp fragment that contains the leader sequence encoding the signal peptide from the sacB gene of B. subtilis was introduced behind the P43 promoter. Different Bg1I restriction sites serve as insertion sites for heterologous genes to be expressed.

Expression of human trypsin I was confirmed by measurement of the proteolytic aciticity in supernatant of cells containing the vector with the gene in comparison to a negative control. A peptide including an arginine cleavage site was chosen as a substrate. The peptide was N-terminally biotinylated and labeled with a fluorophore at the C-terminus. After incubation of the peptide with culture supernatant streptavidin was added. Uncleaved peptide associate with streptavidin and lead to a high read out value while cleavage results in low read out values. FIG. 11 shows the time course of a proteolytic digestion of B. subtilis cells containing the vector with the trypsin I gene in comparison to B. subtilis cells containing the vector without the trypsin I gene (negative control).

As a further confirmation of expression of the protease, supernatants of cells containing the vector with the gene and control cells were analyzed by polyacrylamid gel electrophoreses and subsequent western blot using an antibody specific to the target protease. The procedure was performed according to standard methods (Sambrook, J. F; Fritsch, E. F.; Maniatis, T.; Cold Spring Harbor Laboratory Press, Second Edition, 1989, New York). FIG. 8 confirms expression of the protein only in the cells harbouring the vector with the gene for trypsin.

### Example III

Providing a Scaffold Protein

In this example, human trypsin I was used as the scaffold protein. The gene was either used in its natural form, or, alternatively, was modified to result in a scaffold protein with increased catalytic activity or further improved characteristics.

The modification was done by random modification of the gene, followed by expression of the enzyme and subsequent selection for increased activity. First, the gene was PCR amplified under error-prone conditions, essentially as described by Cadwell, R. C and Joyce, G. F. (PCR Methods Appl. 2 (1992) 28-33). Error-prone PCR was done using 30 pmol of each primer, 20 nmol dGTP and dATP, 100 nmol dCTP and dTTP, 20 fmol template, and 5 U Taq DNA polymerase in 10 mM Tris HCl pH 7.6, 50 mM KCl, 7 mM MgCl2, 0.5 mM MnCl2, 0.01% gelatin for 20 cycles of 1 min at 94° C., 1 min at 65° C. and 1 min at 72° C. The resulting DNA library was purified using the Qiaquick PCR Purification Kit following the suppliers' instructions. The PCR product was digested with the restriction enzyme Bg1I and purified. Afterwards, the PCR product was ligated into the E. coli-B. subtilis shuttle vector described above which was digested with Bg1I and dephosphorylated. The ligation products were transformed into E. coli, amplified in LB, and the plasmids were purified using the Qiagen Plasmid Purification Kit following the suppliers' instructions. Resulting plasmids were transformed into B. subtilis cells.

Alternatively, or in addition to random mutagenesis, variants of the gene were statistically recombined at homologous positions by use of the Recombination Chain Reaction, essentially as described in WO 0134835. PCR products of the genes encoding the protease variants were purified using the QIAquick PCR Purification Kit following the suppliers' instructions, checked for correct size by agarose gel electrophoresis and mixed together in equimolar amounts. 80 μg of this PCR mix in 150 mM TrisHCl pH 7.6, 6.6 mM MgCl₂ were heated for 5 min at 94° C. and subsequently cooled down to 37° C. at 0.05° C./s in order to re-anneal strands and thereby produce heteroduplices in a stochastic manner. Then, 2.5 U Exonuclease III per μg DNA were added and incubated for 20, 40 or 60 min at 37° C. in order to digest different lengths from both 3′ ends of the heteroduplices. The partly digested PCR products were refilled with 0.6 U Pfu polymerase per μg DNA by incubating for 15 min at 72° C. in 0.17 mM dNTPs and Pfu polymerase buffer according to the suppliers' instructions. After performing a single PCR cycle, the resulting DNA was purified using the QIAquick PCR Purification Kit following the suppliers' instructions, digested with Bg1I and ligated into the linearized vector. The ligation products were transformed into E. coli, amplified in LB containing ampicillin as marker, and the plasmids were purified using the Qiagen Plasmid Purification Kit following the suppliers' instructions. Resulting plasmids were transformed into B. subtilis cells.

### Example IV

Insertion of SDRs into the Protein Scaffold of Human Trypsin I and Generation of an Engineered Proteolytic Enzyme with Specificity for a Peptide Substrate Having the Sequence KKWLGRVPGGPV

In order to create insertion sites for SDRs in human trypsin I, two pairs of different restriction sites were introduced into the gene at sites that were identified as potential SDR sites (see Example I above) without changing the amino acid sequence. The insertion of the restriction sites was done by overlap extension PCR. Primers restr1 and restr2 were used for the introduction of SacII and BamHI restriction sites, restr3 and restr4 were used for the introduction of KpnI and NheI restriction sites. The sequences of the primers were as follows:

Binding site for restr1 and restr2 and the corresponding amino acid sequence (SEQ ID NO:54):

5′-GGTGGTATCAGCAGGCCACTGCTACAAGTCCCGCATCCAGGT-3′
V V S A G H C Y K S R I Q

Forward primer restr1 (SEQ ID NO:56):

5′-GGTGGTATCCGCGGGCCACTGCTACAAGTCCCGGATCCAGGT-3′

Reverse primer restr2 (SEQ ID NO:57):

5′-ACCTGGATCCGGGACTTGTAGCAGTGGCCCGCGGATACCACC-3′

Binding site for restr3 and restr4 and the corresponding amino acid sequence (SEQ ID NO:58):

5′-CCACTGGCACGAAGTGCCTCATCTCTGGCTGGGGCAACACTGCGA
GCTCT-3′
T G T K C L I S G W G N T A S S

Forward primer restr3 (SEQ ID NO:60):

5′-CCACTGGCACGAAGTGCCTCATCTCTGGCTGGGGCAACACTGCGAGC
TCT-3′

Reverse primer restr4 (SEQ ID NO:61):

5′-AGAGCTAGCAGTGTTGCCCCAGCCAGAGATGAGGCACTTGGTACCAG
TGG-3′

In a first overlap extension PCR, the SacII/BamHI sites were introduced, enabling to insert SDR1, and in a second overlap extension PCR the KpnI/NheI sites, enabling the insertion of SDR2. The product of the overlap extension PCR was amplified using primers pUC-forward and pUC-reverse. The sequences of pUC-forward and pUC-reverse are as follows:

pUC-forward (SEQ ID NO:62):

5′-GGGGTACCCCACCACCATGAATCCACTCCT-3′

pUC-reverse (SEQ ID NO:63):

5′-CGGGATCCGGTATAGAGACTGAAGAGATAC-3′

The restriction sites generated thereby were subsequently used to insert defined or random oligonucleotides into the SDR1 and SDR2 insertion sites by standard restriction and ligation methods. Typically, two complementary synthetic 5′-phosphorylated oligonucleotides were annealed and ligated into a vector carrying the modified human trypsin I gene that was cleaved with the respective restriction enzymes. Oligonucleotides encoding SDR1 were inserted via the SacII/BamHI sites whereas oligonucleotides encoding SDR2 were inserted via the KpnI/NheI sites. For each insertion an oligonucleotide pair according to the following general sequences was used ([P] indicating 5′-phosphorylation, N and X indicating any nucleotide or amino acid residue, respectively):

oligox-SDR1f (SEQ ID NO:64):

5′-[P]-GGGCCACTGCTACNNNNNNNNNNNNNNNNNNAAGTCCCG-3′

oligox-SDR1r (SEQ ID NO:66):

3′-CGCCCGGTGACGATGNNNNNNNNNNNNNNNNNNTTCAGGGCCTAG-
[P]-5′
G H C Y X X X X X X K S

oligox-SDR2f (SEQ ID NO:67):

| | |
|---|---|
| 5′-[P]-CAAGTGCCTCATCTCTGGCTGGGGCAACNNNNNNNNNNNNNNNACTG-3′ | (SEQ ID NO:69) |
| oligox-SDR2r: | |
| 3′-CATGGTTCACGGAGTAGAGACCGACCCCGTTGNNNNNNNNNNNNNNNTGA | |
| CGATC-[P]-5′ | |
| K C L I S G W G N X X X X X T | |

As an alternative to the above method, a PCR based method was used for the integration of random-sequences into the SDR1 and SDR2 insertion sites in the modified human trypsin I. For each SDR, one primer was used where the SDR region is fully randomized. Sequences of the primers were as follows (N=A/C/G/T, B=C/G/T, V=A/C/G):

Primer SDR1-mutnnb-forward (SEQ ID NO:70):

5′-TGGTATCCGCGGGCCACTGCTACNNBNNBNNBNNBNNBNNBAAGTCC
CGGATCCAGGTG-3′

Primer SDR2-mutnnb-reverse (SEQ ID NO:71):

5′-GGCGCCAGAGCTAGCAGTVNNVNNVNNVNNVNNGTTGCCCCAGCCAG
AGATG-3′

The codon NNB, or VNN in the reverse strand, allows all 20 amino acids to made, but reduces the probability of encoding a stop codon from 0.047 to 0.021.

As a further alternative, after identification of SDRs that lead to increased specificity, these SDRs were used as templates for further randomization. Thereby, random peptide sequences were inserted that were partially randomized at each position and partially identical at each position to the original sequence.

As an example, random peptide sequences that have in approximately 1 of 3 cases the template amino acid residue and in approximately 2 of 3 cases any other amino acid residue at each position were inserted into the two SDR insertion sites of the modified human trypsin I. For this purpose, primers that contain at each nucleotide position of the SDR approximately 70% of the template bases and 30% of a mixture of the three other bases were used.

With each primer pair a PCR was performed under standard conditions using the human trypsin I gene as template. The resulting DNA was purified using the QIAquick PCR Purification Kit following the suppliers' instructions and digested with SacII and NheI. After digestion the DNA was purified and ligated into the SacII and NheI digested and dephosphorylayted vector. The ligation products were transformed into E. coli, amplified in LB containing the respective marker, and the plasmids were purified using the Qiagen Plasmid Purification Kit following the suppliers' instructions. Resulting plasmids were transformed into B. subtilis cells. These cells were then separated to single cells, grown to clones, and after expression of the protease gene screened for proteolytic activity.

The following substrates were employed for screening for proteolytic activity (SEQ ID NOs:76 and 77):

| | |
|---|---|
| substrate A | L L W L G R V V G G P V |
| substrate B | K K W L G R V P G G P V |

Protease variants were screened on substrate B at complexities of 106 variants by confocal fluorescence spectroscopy. The substrate was a peptide biotinylated at the N-terminus and fluorescently labeled at the C-terminus. After incubation of the peptide with supernatant of cells expressing different variants of the protease, streptavidin is added and the samples are analysed by confocal fluorimetry. The low concentration of the peptide (20 nM) leads to a preferential cleavage by proteases with a high k_{cat}/K_{M} value, i.e. proteases with high specificity towards the target sequence.

Variants selected in the screening procedure were further evaluated for their specificity towards substrate B and closely related substrate A by measuring time courses of the proteolytic digestion and determining the rate constants which are proportional to the k_{cat}/K_{M} values. Clearly, compared to the human trypsin that was used as scaffold protein, the specific activity of variants 1 and 2 is shifted (SEQ ID NOs: 2 and 3, respectively) towards substrate B. Variant 3 (SEQ ID NO:4), on the other hand, serves as a negative control with similar activities as the human trypsin I. Sequencing of the genes of the three variants revealed the following amino acid sequences in the SDRs.

Table 2: Sequences of the Two SDRs in Three Different Variants Selected for Specific Hydrolysis of Substrate B (SEQ ID NOs:78-83).

| | |
|---|---|
| SDR1 | SDR2 |

| | | |
|---|---|---|
| Trypsin | — — — — — — | — — — — — |
| Variant 1 | D A V G R D | T I T N S |
| Variant 2 | N G R D L E | V R G T W |
| Variant 3 | G F V M F N | R S P L T |

In a further experiment a pool of variants containing different numbers of SDRs per gene were screened for increased specificity using a mixture of the defined substrate and pepton as a competing substrate. Variants containing one or two SDRs per gene have been analyzed further. As a measure for the specificity the activity in the peptide cleavage assay was compared with and without the presence of the competing substrate. The concentration of the competing substrate was 10 mg/ml. Under these conditions, unspecific proteases show, compared to specific proteases, a stronger decrease in activity with increasing competitor concentrations (range between 0 and 100 mg/ml). The ratio of proteolytic activity with and without substrate is a quantitative measure for the specificity of the proteases. FIG. 9 shows the relative activities with and without competing substrate. Human trypsin I that was used as the scaffold protein and two variants, one containing only SDR2, and one containing both SDRs, were compared. The specificity of the variant with both SDRs is by a factor of 2.5 higher than that of the variant with SDR2 only, confirming that there is a direct relation between the number of SDRs and the quantitative specificity of resulting engineered proteolytic enzymes.

### Example V

Generation of an Engineered Proteolytic Enzyme that Specifically Inactivates Human TNF-Alpha

Human trypsin alpha I or a derivative comprising one or more of the following amino acid substitutions E56G; R78W; Y13IF; A146T; Cl 83R was used as protein scaffold for the generation of an engineered proteolytic enzyme with high specificity towards human TNF-alpha. The identification of SDR sites in human trypsin I or derivatives thereof was done as described above. Two insertion sites within the scaffold were choosen for SDRs. The protease variants containing two inserts with different sequences and also the human trypsin I itself with no inserts were expressed in a Bacillus subtilis cells. The variant protease cells were separated to single cell clones and the protease expressing variants were screened for proteolytic activity on peptides with the desired target sequence of TNF-alpha. The activity of the protease variants was determined as the cleavage rate of a peptide with the desired target sequence of TNF-alpha in the absence and presence of competitor substrate. The specificity is expressed as the ratio of cleavage rates in the presence and absence of competitor (FIG. 14).

TABLE 3

Relative specificity of variants of engineered
proteolytic enzymes with different SDR
sequences in absence and presence of competitor
substrate (SEQ ID NOs:84-95).

| | | |
|---|---|---|
| k with comp./ | | |
| k without comp. | Seq of SDR 1 | Seq of SDR 2 |

| | | | |
|---|---|---|---|
| scaffold (no SDRs) | 0.092 | — | — |
| variant a | 0.130 | RPWDPS | VHPTS |
| variant b | 0.187 | GFVMFN | RSPLT |
| variant c | 0.235 | EIANRE | RGART |
| variant d | 0.310 | KAVVGT | RTPIS |
| variant e | 0.374 | VNIMAA | TTARK |
| variant f | 0.487 | AAFNGD | RKDFW |

The antagonistic effect of three inventive protease variants on human TNF-alpha is shown in FIG. 15. By the use of the variants, the induction of apoptosis is almost completely eliminated indicating the anti-inflammatory efficacy of the inventive proteases to initiate TNF-alpha break down. TNF-alpha has been incubated with concentrated supernatant from cultures expressing the variants i to iii for 2 hours. The resulting TNF-alpha has been incubated with non-modified cells for 4 hours. The effect of the remaining TNF-alpha activity was determined as the extent of apoptosis induction by detection of activated caspase-3 as marker for apoptotic cells. For the controls either no protease was added with the human TNF-alpha (dead cells) or buffer instead of human TNF-alpha (live cells) was used, respectively. An analogous experiment is shown in FIG. 16 using purified variant xiii. TNF-alpha was incubated with different concentrations of the purified inventive protease variant.

To demonstrate the specificity of the inventive protease variants, proteins from human blood serum or purified human TNF-alpha have been incubated with human trypsin I or the inventive engineered proteolytic enzyme variants, respectively. Here, variant x corresponds to Seq ID No: 75 comprising the same SDRs as variant f, i.e. SDRs according to Seq ID No. 89 (SDR1) and 95 (SDR2). Variants xi and xii correspond to derivatives thereof comprising the same SDR sequences. Remaining intact protein was was determined as a function of time. While the variants as well as human trypsin I digest human TNF-alpha, only trypsin shows activity on serum protein (FIG. 17 a and b). This demonstrates the high TNF-alpha specificity of the inventive proteolytic enzymes and indicates their safety and accordingly their low side effects for therapeutic use.

### Example VI

Generation of an Engineered Proteolytic Enzyme that Specifically Hydrolysis Human VEGF

Human trypsin I was used as protein scaffold for the generation of an engineered proteolytic enzyme with high specificity towards human VEGF. The identification of SDR sites in human trypsin I was done as described above. Two insertion sites within the scaffold were choosen for SDRs. The protease variants containing two inserts with different sequences were expressed in Bacillus subtilis cells. The variant protease cells were separated to single cell clones and the protease expressing variants were screened as described above. The activity of the protease variants was determined as the rate of VEGF cleavage. 4 μg of recombinant human VEGF165 was incubated with 0.18 μg of purified protease in PBS/pH 7.4 at room temperature. Aliquots were taken at the indicated time points and analysed on a polyacrylamide gel. The extend of cleavage was quantified by densitometric analysis of the bands. The activity is plotted over incubation time in FIG. 18. Specific cleavage was controlled by further SDS polyacrylamide gel analyses.

1. A method for treatment of a disease in a patient connected with a specific target substrate, which comprises administering the patent a suitable amount of a protease with defined specificity for said specific target substrate.

2. The method of claim 1, wherein the protease hydrolizes the target substrate and thereby eliminates or reduces one or more biological activities or physico-chemical properties or pharmacological properties of the target protein.

3. The method of claim 1, wherein the protease hydrolizes the target substrate and thereby activates or increases one or more biological activities or physico-chemical properties or pharmacological properties of the target protein.

4. The method of claim 1, wherein the protease hydrolizes the target substrate and thereby adds one or more biological activities or physico-chemical properties or pharmacolocical properties to the target protein.

5. The method of claim 1, wherein the target substrate hydrolyzed by the protease is a soluble protein.

6. The method of claim 5, wherein the soluble protein is selected from the group consisting of cytokines, hormones, toxins, enzymes, structural proteins and immunoglobulins.

7. The method of claim 6, wherein the cytokines are selected from the group consisting of the TNF-superfamily proteins, interleukines, interferons, chemokines and growth factors.

8. The method of claim 6, wherein the enzymes are selected from the group consisting of oxidoreductases, transferases, hydrolases, lyases, isomerases and ligases.

9. The method of claim 6, wherein the structural proteins are collagens.

10. The method of claim 1, wherein the target substrate hydrolyzed by the protease is a membrane associated protein.

11. The method of claim 10, wherein the membrane associated protein is selected from the group consisting of single pass transmembrane proteins, multipass transmembrane proteins, lipid-anchored membrane proteins and GPI-anchored membrane proteins.

12. The method of claim 11, wherein the multipass transmembrane proteins are selected from the group consisting of G-protein coupled receptors, ion channels and transporters.

13. The method of claim 7, wherein the target substrate hydrolyzed by the protease is a TNF-superfamily protein.

14. The method of claim 13, wherein the protease is capable of hydrolysing peptide bonds in human tumor necrosis factor-alpha (hTNF-α, SEQ ID NO:96) or related molecules of the same structure class.

15. The method of claim 12, wherein the protease is capable of hydrolizing the peptide bonds between the positions selected from the group consisting of 31/32, 32/33, 44/45, 45/46, 87/88, 128/129, 130/131, 140/141 and/or 141/142 of hTNF-α.

16. The method of claim 14, wherein the protease is capable of hydrolizing the peptide bonds between the positions selected from the group consisting of 31/32, 32/33 and/or 45/46 of hTNF-α or between analogous positions in said related molecules.

17. The method of claim 13, wherein the medicament is suitable for the treatment of diseases selected from the group consisting of rheumatoid arthritis, inflammatory bowel diseases, psoriasis, Crohn's disease, Ulcerative colitis, diabetes type II, classical Hodgkin's Lymphoma (cHL), Grave's disease, Hashimoto's thyroiditis, Sjogren's syndrome, systemic lupus erythematosus, multiple sclerosis, Systemic inflammatory response syndrome (SIRS), multiple organ dysfunction syndrome (MODS), eosinophilia, neurodegenerative disease, stroke, closed head injury, encephalitis, CNS disorders, asthma, rheumatoid arthritis, sepsis, vasodilation, intravascular coagulation, multiple organ failure and other diseases connected with hTNF-α.

18. The method of claim 14, wherein the protease is derived from a serine protease of the structural class S1

19. The method of claim 18, wherein the protease is derived from human trypsin I (SEQ ID NO:1).

20. The method of claim 14, wherein the protease is selected from the group consisting of SEQ ID NO:74 and 75 and is capable of hydrolysing hTNF-α at positions 31/32 and/or 32/33.

21. The method of claim 7, wherein the target substrate hydrolyzed by the protease is a growth factor.

22. The method of claim 21, wherein the protease is capable of hydrolysing peptide bonds in human Vascular endothelial growth factor (hVEGF) or related molecules of the same structural class.

23. The method of claim 22, wherein the protease is capable of hydrolyzing the peptide bonds between the positions selected from the group of positions consisting of 16/17, 19/20, 23/24, 34/35, 41/42, 56/57, 62/63, 63/64, 64/65, 65/66, 82/83, and 84/85 of hVEGF (SEQ ID NO:103).

24. The method of claim 22, wherein the protease is capable of hydrolyzing the peptide bonds between the positions selected from the group of positions consisting of 23/24, 41/42, 63/64, 82/83 and/or 84/85 of hVEGF (SEQ ID NO: 103), or between analogous positions in related molecules.

25. The method of claim 21, wherein the medicament is suitable for the treatment of diseases selected from the group consisting of all solid tumors and metastatic solid tumors, inflammatory breast cancer and other diseases connected with hVEGF.

26. The method of claim 1, wherein the protease is an engineered protease.

27. The method of claim 26, wherein the engineered protease is characterized by a combination of the following components:
(a) a protein scaffold capable to catalyze at least one chemical reaction on at least one target substrate, and
(b) one or more specificity determining regions (SDRs) located at sites in the protein scaffold that enable the resulting engineered protein to discriminate between at least one target substrate and one or more different substrates, and wherein the SDRs are essentially synthetic peptide sequences.

28. The method of claim 27, wherein the SDRs (b) have a length between one and 50 amino acid residues.

29. The method of claim 28, wherein the SDRs (b) have a length between 2 and 20 amino acid residues.

30. The method of claim 28, wherein the SDRs (b) have a length between 2 and 10 amino acid residues.

31. The method of claim 28, wherein the SDRs (b) have a length between 3 and 8 amino acid residues.

32. The method of claim 27, wherein the number of SDRs is at least one.

33. The method of claim 32, wherein the number of SDRs is more than one.

34. The method of claim 32, wherein the number of SDRs is between two and eleven.

35. The method of claim 32, wherein the number of SDRs is between two and six.

36. The method of claim 27, wherein the protein scaffold (a) is comprised of one or more polypeptide segments being derived from same or different proteins encoded by a gene selected from the group of genes of viral, prokaryotic and eukaryotic origin.

37. The method of claim 27, wherein the protein scaffold (a) is comprised of one or more polypeptide segments being derived from same or different native enzymes, mutated variants or truncated derivates thereof.

38. The method of claim 27, wherein the protein scaffold (a) is comprised of one or more polypeptide segments being derived from same or different mammalian enzymes.

39. The method of claim 38, wherein the mammalian enzymes are human enzymes.

40. The method of claim 27, wherein the protein scaffold (a) is derived from a protease selected from the group consisting of aspartic, cysteine, serine, metallo and threonine proteases.

41. The method of claim 40, wherein the protein scaffold (a) is derived from a serine protease of the structural class selected from the group consisting of S1, S8, S11, S21, S26, S33 and S51.

42. The method of claim 41, wherein the protein scaffold (a) is derived from a serine protease of the structural class selected from the group consisting of S1 and S8.

43. The method of claim 40, wherein the protein scaffold (a) is derived from a cysteine protease of the structural class selected from the group consisting of C1, C2, C4, C10, C14, C19, C47, C48 and C56.

44. The method of claim 43, wherein the protein scaffold (a) is derived from a cysteine protease of the structural class C14.

45. The method of claim 40, wherein the protein scaffold (a) is derived from an aspartic protease of the structural class selected from the group consisting of A1, A2 and A26.

46. The method of claim 45, wherein the protein scaffold (a) is derived from an aspartic protease of the structural class A1.

47. The method of claim 40, wherein the protein scaffold (a) is derived from a metalloprotease of the structural class selected from the group consisting of M4 and M10.

48. The method of claim 27, wherein the protein scaffold (a) is derived from a serine protease of the structural class S1.

49. The method of claim 48, wherein the SDRs are located at one or more positions selected from the group of positions that correspond structurally or by amino acid sequence homology to the regions 18-25, 38-48, 54-63, 73-86, 122-130, 148-156, 165-171 and 194-204 in human trypsin I having the amino acid sequence shown in SEQ ID NO:1.

50. The method of claim 49, wherein the SDRs are located at one or more positions selected from the group of positions that correspond structurally or by amino acid sequence homology to the regions 20-23, 41-45, 57-60, 76-83, 125-128, 150-153, 167-169 and 197-201 in human trypsin I having the amino acid sequence shown in SEQ ID NO:1.

51. The method of claim 27, wherein the protein scaffold (a) is derived from the serine protease trypsin.

52. The method of claim 51, wherein the serineprotease trypsin is human trypsin I having the amino acid sequence shown in SEQ ID NO: 1 or a derivative thereof.

53. The method of claim 52, wherein the serine protease trypsin has the amino acid sequence SEQ ID NO: 1 comprising one or more of the amino acid substitutions selected from the group consisting of E56G, R78W, Y131F, A146T and C183R.

54. The method of claim 52, wherein at least one of two SDRs are located in the scaffold, a first SDR having a length of up to 6 amino acids and been inserted between residues 42 and 43, and a second SDR having a length of up to 5 amino acids and been inserted between residues 123 and 124, the numbering being relative to human trypsin having the amino acid sequence shown in SEQ ID NO: 1.

55. The method of claim 54, which comprises one of the peptide sequences selected from the group consisting of SEQ ID NO: 72, 78, 79, 80, 84, 85, 86, 87, 88, and 89 is inserted as the first SDR between residues 42 and 43.

56. The method of claim 54, which comprises one of the peptide sequences selected from the group consisting of SEQ ID NO: 73, 81, 82, 83, 90, 91, 92, 93, 94, and 95 is inserted as the second SDR between residues 123 and 124.

57. The method of claim 27, where the engineered enzyme comprises an amino acid sequence selected from the group consisting of SEQ ID NO: 74 and SEQ ID NO: 75.

58. The method of claim 27, wherein the protein scaffold (a) is derived from a serine protease of the structural class S8.

59. The method of claim 58, wherein the SDRs are located at one or more positions selected from the group of positions that correspond structurally or by amino acid sequence homology to the regions 6-17, 25-29, 47-55, 59-69, 101-111, 117-125, 129-137, 139-154, 158-169, 185-195 and 204-225 in subtilisin E from Bacillus subtilis having the amino acid shown in SEQ ID NO:7.

60. The method of claim 59, wherein the SDRs are located at one or more positions selected from the group of positions that correspond structurally or by amino acid sequence homology to the regions 59-69, 101-111, 129-137, 158-169 and 204-225 in subtilisin E for Bacillus subtilis.

61. The method of claim 46, wherein the SDRs are located at one or more positions selected from the group of positions that correspond structurally or by amino acid sequence homology to the regions 6-18, 49-55, 74-83, 91-97, 112-120, 126-137, 159-164, 184-194, 242-247, 262-267 and 277-300 in human pepsin having the amino acid sequence shown in SEQ ID NO: 11.

62. The method of claim 61, wherein the SDRs are located at one or more positions selected from the group of positions that correspond structurally or by amino acid sequence homology to the regions 10-15, 75-80, 114-118, 130-134, 186-191 and 280-296 in human pepsin.

63. The method of claim 44, wherein the SDRs are located at one or more positions selected from the group of positions that correspond structurally or by amino acid sequence homology to the regions 78-91, 144-160, 186-198, 226-243 and 271-291 in human caspase 7 having the amino acid sequence of SEQ ID NO: 14.

64. The method of claim 63, wherein the SDRs are located at one or more positions selected from the group of positions that correspond structurally or by amino acid sequence homology to the regions 80-86, 149-157, 190-194 and 233-238 of human caspase 7.

65. The method of claim 27, wherein the protease comprises at least one further proteinacious component.

66. The method of claim 65, wherein the proteinacious component is selected from the group consisting of binding domains, receptors, antibodies, regulation domains, pro-sequences, and fragments thereof.

67. The method of claim 27, wherein the protease comprises at least one further functional component.

68. The method of claim 67, wherein the functional component is selected from the group consisting of polyethylenglycols, carbohydrates, lipids, fatty acids, nucleic acids, metals, metal chelates, and fragments or derivatives thereof.

69. The method according to claim 27, wherein the protease is obtainable by a method comprising at least the following steps:
(a) providing a protein scaffold which catalyzes at least one chemical reaction on at least one target substrate,
(b) generating a library of enzymes or isolated enzymes by combining the protein scaffold from step (a) with variants of one or more fully or partially random synthetic oligonucleotide sequences encoding synthetic peptide sequences at sites in the protein scaffold that enable the resulting enzyme to discriminate between at least one target substrate and one or more different substrates, expressing said enzyme, and
(c) selecting out of the (library of) enzymes generated in step (b) one or more enzymes that have defined specificities towards at least one target substrate.

70. An in vivo or in vitro diagnostic method which comprises the use of a protease of claim 1.

71. A pharmaceutical composition comprising one or more enzymes of claim 1.

72. The pharmaceutical composition of claim 71, which optionally comprises pharmaceutically acceptable carrier(s), excipient(s) and/or auxiliary agent(s).

73. A diagnostic composition comprising one or more enzymes of claim 1.

74. The diagnostic composition of claim 73, which optionally comprises diagnostically acceptable carrier(s), excipient(s) and/or auxiliary agent(s).

75. A method for cleaving a target substrate as defined in claim 1 in vivo or in vitro, which comprises contacting the target substrate with a protease as defined in claim 1.
